(19) United States
(12) Patent Application Publication
(10) Pub. No.: US 2020/0046816 A1
(43) Pub. Date: Feb. 13, 2020

(54) Compositions and Methods for Antigen-Specific Tolerance

(71) Applicants: Northwestern University, Evanston, IL (US); Myelin Repair Foundation, Inc., Saratoga, CA (US)

(72) Inventors: Stephen D. Miller, Oak Park, IL (US); Michael A. Pleiss, Sunnyvale, CA (US); Daniel R. Getts, Northbrook, IL (US); Aaron Martin, Evanston, IL (US)

(21) Appl. No.: 16/352,604

(22) Filed: Mar. 13, 2019

Related U.S. Application Data
(63) Continuation of application No. 14/405,751, filed on May 26, 2015, now abandoned.
(60) Provisional application No. 61/656,487, filed on Jun. 6, 2012.

(51) Int. Cl. A61K 39/00; A61K 47/69; A61K 47/64; C12N 5/078; C12N 5/00; A61K 38/17; A61K 38/18; A61K 38/20

(52) U.S. Cl. CPC A61K 39/001; A61K 2035/122; A61K 47/6901; A61K 47/646; A61K 47/6933; A61K 47/6937; C12N 5/0648; C12N 5/0006; A61K 38/1709; A61K 38/1761; A61K 38/1841; A61K 38/2013; A61K 38/2066; A61K 2039/577; A61K 2039/545; A61K 2039/5154; A61K 39/0008

(57) ABSTRACT

The present invention provides compositions and methods for inducing antigen-specific tolerance in a subject. In one embodiment, the present invention provides a composition comprising an apoptotic body and an epitope of an antigen. Also provided herein are methods of preparing and administering the composition. The composition and methods provided herein can induce antigen-specific tolerance in a subject.

### CROSS REFERENCE TO RELATED APPLICATIONS

This Application is a Continuation of application Ser. No. 14/405,751, filed May 26, 2015, now abandoned, which is a U.S. National Phase Application of PCT/US2013/044616, which claims the priority benefit of U.S. Provisional Application 61/656,487, filed Jun. 6, 2012. The entire contents of these applications are incorporated herein by reference in their entirety.

### RELATED APPLICATIONS

This application claims the benefit of U.S. Provisional Application No. 61/656,487, filed Jun. 6, 2012, which is herein incorporated by reference in its entirety.

### STATEMENT AS TO FEDERALLY SPONSORED RESEARCH

This invention was made with government support under US National Institutes of Health grants NS026543 and EB013198. The government has certain rights in the invention.

### BACKGROUND

The first step leading to the initiation of an immune response is thought to be the recognition of antigen fragments presented in association with major histocompatibility complex (MHC) molecules. Recognition of antigens can occur directly when the antigens are associated with the MHC on the surface of foreign cells or tissues, or indirectly when the antigens are processed and then associated with the MHC on the surface of professional antigen presenting cells (APC). Resting T lymphocytes that recognize such antigen-MHC complexes become activated via association of these complexes with the T cell receptor (Jenkins et al., J. Exp. Med. 165, 302-319, 1987; Mueller et al., J. Immunol. 144, 3701-3709, 1990).

A living organism generally does not display immune response to a self-composing antigen. This is called natural or innate immunological tolerance. On the other hand, even if an antigen is originally heterogeneous to a living organism, it may not react to the immune response which is displayed on dosing of the antigen, depending on when it is dosed, how it is dosed and in what form it is dosed. This is called acquired tolerance. If T cells are only stimulated through the T cell receptor, without receiving an additional costimulatory signal, they become nonresponsive, anergic, or die, resulting in downmodulation of the immune response, and tolerance to the antigen. (Van Gool et al., Eur. J. Immunol. 29(8):2367-75, 1999; Koenen et al., Blood 95(10):3153-61, 2000). However, if the T cells receive a second signal, termed costimulation, T cells are induced to proliferate and become functional (Lenschow et al., Annu. Rev. Immunol. 14:233, 1996). The self/non-self recognition is thought to occur at the interaction level of antigen presenting cells (e.g. dendritic cells or macrophages), and T lymphocytes.

Autoimmune Disease (AD) is a major health problem. The National Institutes of Health (NIH) estimates up to 23.5 million Americans suffer from autoimmune disease and that the prevalence is rising. In comparison, cancer affects up to 9 million and heart disease up to 22 million. NIH estimates annual direct health care costs for AD to be in the range of $100 billion (“The Cost Burden of Autoimmune Disease: The Latest From in the War on Healthcare Spending”, AARDA, NCAPG; NIAID). In comparison, cancer costs are $57 billion (NIH; ACS), and heart and stroke costs are $200 billion (NIH; AHA). NIH research funding for AD in 2003 came to $591 million. In comparison, cancer funding came to $6.1 billion; and heart and stroke, to $2.4 billion. The NIH Autoimmune Diseases Research Plan states; “Research discoveries of the last decade have made autoimmune research one of the most promising areas of new discovery.”

80-100 different autoimmune diseases have been identified and at least 40 additional diseases are suspected of having an autoimmune basis. These diseases are chronic and can be life-threatening. Autoimmune disease is one of the top 10 leading causes of death in female children and women in all age groups up to 64 years of age. A close genetic relationship exists among autoimmune disease, explaining clustering in individuals and families as well as a common pathway to disease. Symptoms associated with autoimmune diseases cross many specialties and can affect all body organs. Understanding how to modulate immune system activity will benefit transplant recipients, cancer patients, AIDS patients and infectious disease patients.

An estimated 50 million Americans suffer from all types of allergies (1 in 5 Americans) including indoor/outdoor, food & drug, latex, insect, skin and eye allergies. Allergy prevalence overall has been increasing since the early 1980s across all age, sex and racial groups (“CDC Fast Facts A-Z,” Vital Health Statistics, 2003). Allergy is the 5^{th} leading chronic disease in the U.S. among all ages, and the 3^{rd} most common chronic disease among children under 18 years old (“Chronic Conditions: A Challenge for the 21^{st} Centrury,” National Academy on an Aging Society, 2000). Many people with allergies usually have more that one type of allergy. Approximately 40 million Americans have indoor/outdoor allergies (allergic rhinitis; seasonal/perennial allergies; hay fever; nasal allergies) as their primary allergy. Approximately 10 million people are allergic to cat dander, the most common pet allergy. The most common indoor/outdoor allergy triggers are: tree, grass and weed pollen; mold spores; dust mite and cockroach allergen; and cat, dog and rodent dander. Approximately 7% of allergy sufferers have skin allergies (atopic dermatitis; eczema; hives; urticaria; contact allergies) as their primary allergy. Plants such as poison ivy, oak and sumac are the most common skin allergy triggers. However, skin contact with cockroach and dust mite allergen, certain foods or latex may also trigger symptoms of skin allergy. Approximately 6% of allergy sufferers have food/drug allergies as their primary allergy. Food allergy is more common among children than adults. 90% of all food allergy reactions are caused by 8 foods: milk, soy, eggs, wheat, peanuts, tree nuts, fish and shellfish. For drug allergies, penicillin is the most common allergy trigger. Approximately 4% of allergy sufferers have latex allergy as their primary allergy An estimated 10% of healthcare workers suffer from latex allergy. Approximately 4% of allergy sufferers have insect allergies as their primary allergy (bee/wasp stings and venomous ant bites; cockroach and dust mite allergen may also cause nasal or skin allergy symptoms). Approximately 4% of allergy sufferers have eye allergies (allergic conjunctivitis; ocular allergies) as their primary allergy, often caused by many of the same triggers as indoor/outdoor allergies.

Allergies are the most frequently reported chronic condition in children, limiting activities for more than 40% of them. Each year, allergies account for more than 17 million outpatient office visits, primarily in the spring and fall; seasonal allergies account for more than half of all allergy visits (“CDC Fast Facts A-Z,” Vital Health Statistics, 2003). Skin allergies alone account for more than 7 million outpatient visits each year (“In Allergy Principles and Practice: 5^{th} Edition, 1998). Food allergies account for 30,000 visits to the emergency room each year and exposure to latex allergen alone is responsible for over 200 cases of anaphylaxis (severe allergic reactions) each year (“Anaphylaxis in the United States,” Archives of Internal Medicine, 2011).

The annual cost of allergies is estimated to be nearly $7 billion. Direct costs accounted for nearly $6 billion ($5.7 billion in medications and $300 million in office visits). For adults, allergies (hay fever) is the 5^{th} leading chronic disease and a major cause of work absenteeism, resulting in nearly 4 million missed or lost workdays each year, and a total cost of more than $700 million in total lost productivity (“Chronic Conditions: A Challenge for the 21^{st} Centrury,” National Academy on an Aging Society, 2000).

Autoimmune diseases, such as multiple sclerosis, psoriasis, rheumatoid arthritis and type 1 diabetes, are the third ranked cause of human morbidity and mortality in the United States. In these disorders, a failure in immune regulation results in T-cell-mediated destruction of self tissues. The pathologic role of T cells in driving autoimmune diseases has resulted in numerous therapies aimed at inactivating T cells. The induction of long-term, durable antigen-specific T-cell tolerance is the ideal therapy, but published ‘tolerance-inducing’ strategies such as T cell epitope-specific peptides, T cell-specific antibodies or co-stimulation blockade have not faired well clinically. Many of the failures were caused by issues associated specifically with the particular target and agent; however, many also involved concerns about safety and marginal efficacy. For example, cytokine release syndrome has been a common issue with the use of monoclonal antibody-based treatments, whereas soluble peptide infusion has induced anaphylactic responses in mouse models.

Conventional clinical strategies for general long-term immunosuppression in disorders associated with an undesired immune response (e.g., autoimmune disease, graft rejection) are based on the long-term administration of broad acting immunosuppressive drugs, for example, signal 1 blockers such as for example cyclosporine A (CsA), FK506 (tacrolimus) and corticosteroids. Long-term use of high doses of these drugs can also have toxic side-effects. Moreover, even in those patients that are able to tolerate these drugs, the requirement for life-long immunosuppressive drug therapy carries a significant risk of severe side effects, including tumors, serious infections, nephrotoxicity and metabolic disorders.

A number of antigen-specific approaches to generate tolerance have previously been tested in autoimmune diseases. Intradermal administration of CGP77116, an altered peptide ligand of MBP₈₃₋₉₉, worsened symptoms in three patients with multiple sclerosis because in at least two of the patients there were increased immune responses to MBP₈₃₋₉₉. Attempts to induce ‘high-zone tolerance’ by i.v. (MBP8298) infusion of a large bolus of peptide recently failed a phase 3 clinical trial in patients with multiple sclerosis. Similarly, in type 1 diabetes, s.c. injection of the 65-kDa isoform of glutamic acid decarboxylase in alum had no effect on disease progression. Mucosal antigen delivery has also shown promise in animal models of multiple sclerosis and type 1 diabetes, but larger clinical trials testing oral and nasal administration of insulin have been ineffective in the prevention or reversal of new-onset type 1 diabetes.

A DNA vaccine, ATX-MS-1467, expresses peptides that are thought to mimic processed myelin antigens and therefore act similarly to glatiramer acetate (GLAT), a random-length polymer of four amino acids (glutamic acid, lysine, alanine and tyrosine) found in MBP, which has been shown to compete with myelin peptides for access to the peptide binding cleft in the MHC complex, promote T_{H}2 cell responses and induce IL-10-producing T_{reg} cells. These effects are not antigen specific, and as such, it may be predicted that the efficacy of ATX-MS-1467 may be similar to that of GLAT, resulting in a 50% reduction in multiple sclerosis relapses in responsive patients.

Thus, to avoid complications of immunosuppression, the ability to tolerize T cells specific for autoantigens and alloantigens remains the desired treatment for a myriad of immune-mediated diseases.

### SUMMARY

There exists a pressing need for compositions and methods for ameliorating the undesirable immune responses effectively. The present invention addresses this need and/or provides related advantages as well.

The present invention provides compositions and methods for inducing antigen-specific tolerance in a subject. In one embodiment, the present invention provides a composition comprising an apoptotic body and an epitope of an antigen. Also provided herein are methods of preparing and administering the composition. The composition and methods provided herein can induce antigen-specific tolerance in a subject.

In a first aspect, the invention relates to a method of inducing antigen-specific tolerance in a subject suffering from or at risk of a condition comprising: administering a composition to said subject, wherein said composition comprises an apoptotic body surrogate and a plurality of immunodominant epitopes associated with one or more antigens suspected to cause said condition, wherein said composition induces tolerance of said at least one or more antigens in said subject. In some embodiments, said one or more antigens acts as an allergen that would otherwise induce T-cell receptor-mediated stimulation in said subject. In some embodiments, said plurality of immunodominant epitopes is from one antigen. In some embodiments, said plurality of immunodominant epitopes is from different antigens and wherein said different antigens act as an allergen that would otherwise induce T-cell receptor-mediated stimulation in said subject. In some embodiments, said different antigens are associated with said condition. In some embodiments, said different antigens are associated with said condition. In some embodiments, said different antigens are associated with said condition and one or more additional conditions. In some embodiments, said conditions comprise different allergies. In some embodiments, said condition is an autoimmune disease, transplant rejection, or allergy. In some embodiments, said condition is multiple sclerosis. In some embodiments, said plurality of immunodominant epitopes is attached to said apoptotic body surrogate. In some embodiments, said plurality of immunodominant epitopes is attached to a plurality of apoptotic body surrogates. In some embodiments, said composition is administered prior to said subject's exposure to said antigen. In some embodiments, said composition is administered subsequent to said subject's exposure to said antigen. In some embodiments, said administering is prior to or concurrent with onset of said condition. In some embodiments, said administering is subsequent to onset of said condition. In some embodiments, said administering prevents relapse of said condition. In some embodiments, said administering of said composition is prior to administration of a therapeutic or vaccine. In some embodiments, said subject has never been exposed to one or more of said antigens. In some embodiments, said subject has previously had an adverse reaction to said one or more antigens.

In another aspect, the invention relates to a method of reducing a hypersensitivity response of a food allergy in a subject comprising: administering a composition comprising an apoptotic body surrogate and an immunodominant epitope of said food to said subject, wherein said composition induces tolerance of said food in said subject thereby reducing the hypersensitivity response of said food allergy in said subject. In some embodiments, said subject's contact with said food would otherwise induce T-cell receptor-mediated stimulation in said subject. In some embodiments, said food is a nut. In some embodiments, said food is a shellfish. In some embodiments, said food comprises gluten or dairy. In some embodiments, said subject has never been exposed to said food. In some embodiments, said subject has previously had an adverse reaction to said food. In some embodiments, said epitope is from an antigen comprising a polypeptide, polynucleotide, carbohydrate, or glycolipid.

In a further aspect, the invention relates to a method of reducing the risk of transplant rejection in a subject comprising: administering a composition comprising an apoptotic body surrogate and an immunodominant epitope of a tissue to be transplanted to said subject, wherein said composition induces tolerance of said tissue in said subject thereby reducing the risk of transplant rejection in said subject. In some embodiments, said tissue acts as an allergen that would otherwise induce T-cell receptor-mediated stimulation in said subject. In some embodiments, said antigen comprises an allogeneic cell extract or endothelial cell antigen. In some embodiments, said administering is performed prior to transplantation of said tissue. In some embodiments, said administering is performed concurrent with or subsequent to transplantation of said tissue. In some embodiments, said epitope is from an antigen comprising a polypeptide, polynucleotide, carbohydrate, or glycolipid.

In a yet further aspect, the invention relates to a method of reducing a hypersensitivity response to a therapeutic in a subject comprising: administering a composition comprising an apoptotic body surrogate and an epitope of a therapeutic, wherein said composition induces tolerance of said therapeutic in said subject thereby reducing said hypersensitivity response to said therapeutic in said subject. In some embodiments, said therapeutic acts as an allergen that would otherwise induce T-cell receptor-mediated stimulation in said subject. In some embodiments, said therapeutic is a small molecule, antibody, nucleic acid, or peptide. In some embodiments, said therapeutic comprises an antibody or fragment thereof. In some embodiments, said administering of said composition is prior to administration of said therapeutic to said subject. In some embodiments, said administering of said composition is concurrent with or subsequent to administration of said therapeutic to said subject. In some embodiments, said subject has never been exposed to said therapeutic. In some embodiments, said subject has previously had an adverse reaction to said therapeutic. In some embodiments, said epitope is from an antigen comprising a polypeptide, polynucleotide, carbohydrate, or glycolipid.

In another aspect, the invention relates to a method of inducing antigen-specific tolerance in a subject suffering from or at risk of hypersensitivity to an antigen comprising: (a) obtaining personalized information of a subject; (b) determining from said personalized information an antigen to which said subject is hypersensitive to; and (c) administering a composition comprising an apoptotic body or apoptotic body surrogate and an epitope of said antigen to said subject, thereby inducing tolerance specific to said antigen in said subject. In some embodiments, said antigen acts as an allergen that would otherwise induce T-cell receptor-mediated stimulation in said subject. In some embodiments, said personalized information comprises medical history, family history, or genotype information of said subject. In some embodiments, said personalized information comprises allergic reaction information, autoimmune disorder records, or inflammatory disorder records of said subject or family members of said subject. In some embodiments, the method further comprises generating said genotype. In some embodiments, said genotype is obtained by a third party. In some embodiments, said genotype comprises a genetic mutation, deletion, insertion, or polymorphism. In some embodiments, said subject is determined to be hypersensitive to one or more additional antigens.

In a further aspect, the invention relates to a method of inducing antigen-specific tolerance in a subject suffering from or at risk of hypersensitivity to an antigen comprising: (a) obtaining a pool of immune cells from a subject; (b) determining from said pool an antigen to which said subject is hypersensitive to; and (c) administering a composition comprising an apoptotic body surrogate and an epitope of said antigen to said subject, thereby inducing tolerance specific to said antigen in said subject. In some embodiments, said antigen acts as an allergen that would otherwise induce T-cell receptor-mediated stimulation in said subject. In some embodiments, said immune cells comprises T-cells. In some embodiments, said determining comprises subjecting said T-cells to a variety of antigens and identifying a T-cell response to an antigen, thereby determining an antigen to which said subject is hypersensitive to. In some embodiments, said T-cells response is assayed by determining T-cell proliferation or cytokine secretion. In some embodiments, said T-cells response is assayed by flow cytometry. In some embodiments, said subject is determined to be hypersensitive to one or more additional antigens.

In yet another aspect, the invention relates to a method of delivering an antigen to a splenic marginal zone of a subject comprising: administering a composition comprising an apoptotic body surrogate and an antigen to a subject, wherein said apoptotic body surrogate is recognized by a macrophage scavenger receptor, and said macrophage scavenger receptor uptakes said antigen in said splenic marginal zone. In some embodiments, said apoptotic body surrogate is cleared from said splenic marginal zone within 24 hours. In some embodiments, said macrophage scavenger receptor is MARCO.

In various aspects, compositions may be delivered orally, nasally, intravenously, intramuscularly, parenterally, or ocularly. Antigens may be coupled to said apoptotic body surrogate by a conjugate molecule. The conjugates may comprise an ethylene or carbodiimide conjugate. In some embodiments, said conjugate is ethylene carbodiimide (ECDI).

In various aspects apoptotic body surrogates may have a size of an apoptotic body, a localization pattern of an apoptotic body, is uptake by a macrophage, or binds Thrombospondin 1, Gas-6, or MFG-E8. Apoptotic body surrogates may comprise a quantum dot, dendrimer, liposome, micelle, nanoparticle or microparticle. Apoptotic body surrogates may be between 5 nm and 10 μm in diameter, In some embodiments, apoptotic body surrogates are less than 10 nm in diameter. In some embodiments, the apoptotic body surrogate is about 500 nm in diameter. Apoptotic body surrogates may be biodegradable. Apoptotic body surrogates may comprise a polyglycolic acid polymer (PGA), polylactic acid polymer (PLA), polysebacic acid polymer (PSA), poly(lactic-co-glycolic) acid polymer (PLGA), poly(lactic-co-sebacic) acid copolymer (PLSA), poly(glycolic-co-sebacic) acid copolymer (PGSA), polylactide co-glycolide (PLG), chitosan, or hyaluronic acid.

In some aspects, expression of IL-10, IL-2 or PD-L1 expression may be induced in subjects.

In various aspects, a plurality of antigens, an apoptotic signaling molecule or an additional anergy promoting agent is administered to subjects in addition to the composition. In some embodiments, said composition comprises said plurality of antigens, apoptotic signaling molecule or additional anergy promoting agent. In some embodiments, said antigen or said apoptotic body surrogate is attached to said plurality of antigens, apoptotic signaling molecule or additional anergy promoting agent. In some embodiments, said apoptotic signaling molecule is annexin-1, annexin-5, milk fat globule-EGF-factor 8 (MFG-E8), calreticulin, phosphatidylserine, CD47, oxidized LDL, Fas-ligand or TNF-alpha. In some embodiments, said additional anergy promoting agent is a cytokine. In some embodiments, said cytokine is IL-10, IL-2 or TGF-β. In some embodiments, the additional anergy promoting agent is administered subsequent the administration of the apoptotic body surrogate. In some embodiments, the additional anergy promoting agent comprises IL-10, IL-2 or TGF-β. In some embodiments, the subsequent administration of the additional anergy promoting agent is at least 1, 2, 3, 4, 5, 6, 7, 10, 12, 14, 21, 28 or more days after the administration of the apoptotic body surrogate.

In a further aspect, the invention relates to a composition for induction of an antigen-specific tolerance in a subject suffering from or at risk of a condition comprising: (a) an apoptotic body surrogate and (b) a plurality of immunodominant epitopes associated with one or more antigens suspected to cause a condition, wherein said composition induces tolerance of said at least one or more antigens in said subject. In some embodiments, said antigen acts as an allergen that would otherwise induce T-cell receptor-mediated stimulation in said subject. In some embodiments, said plurality of immunodominant epitopes is from one antigen. In some embodiments, said plurality of immunodominant epitopes is from different antigens and said plurality of antigens act as allergens that would otherwise induce T-cell receptor-mediated stimulation in said subject. In some embodiments, said different antigens are associated with said condition. In some embodiments, said different antigens are associated with said condition and one or more additional conditions. In some embodiments, said condition is an autoimmune disease, transplant rejection, or allergy. In some embodiments, said condition is multiple sclerosis. In some embodiments, said condition is a food allergy. In some embodiments, said conditions comprise different allergies. In some embodiments, said plurality of immunodominant epitopes is attached to said apoptotic body surrogate. In some embodiments, said plurality of immunodominant epitopes is attached to a plurality of apoptotic body surrogates. In some embodiments, the composition further comprises an apoptotic signaling molecule or additional anergy promoting agent. In some embodiments, said antigen or said apoptotic body surrogate is attached to said apoptotic signaling molecule or additional anergy promoting agent.

In a yet further aspect, the invention relates to a composition for induction of antigen-specific tolerance in a subject suffering from or at risk of a condition comprising: (a) an apoptotic body surrogate, (b) an epitope associated with one or more antigens suspected to cause said condition, and (c) an additional anergy promoting agent within said apoptotic body surrogate, wherein said composition induces tolerance of said antigen in said subject. In some embodiments, said antigen acts as an allergen that would otherwise induce T-cell receptor-mediated stimulation in said subject. In some embodiments, said additional anergy promoting agent is a cytokine. In some embodiments, said cytokine is IL-10, IL-2 or TGF-β. In some embodiments, said additional anergy promoting agent is released from said apoptotic body surrogate. In some embodiments, the composition further comprises an apoptotic signaling molecule. In some embodiments, said antigen or said apoptotic body surrogate is attached to said apoptotic signaling molecule. In some embodiments, said apoptotic signaling molecule is annexin-1, annexin-5, milk fat globule-EGF-factor 8 (MFG-E8), calreticulin, CD47, phosphatidylserine, oxidized LDL, Fas-ligand or TNF-alpha. In some embodiments, said epitope is attached to said apoptotic body surrogate.

In various aspects relating to compositions, said attachment may be by a conjugate molecule. In some embodiments, said conjugate comprises an ethylene or carbodiimide conjugate. In some embodiments, said conjugate is ethylene carbodiimide (ECDI).

The apoptotic body conjugates in various aspects may have a size of an apoptotic body, a localization pattern of an apoptotic body, is uptaken by a macrophage, binds a macrophage scavenger receptor, of binds SRBII or MARCO. In some embodiments, the apoptotic body surrogate comprises a quantum dot, dendrimer, liposome, micelle, nanoparticle or microparticle. In some embodiments, the apoptotic body surrogate is between 5 nm and 10 μm in diameter. In some embodiments, the apoptotic body surrogate is less than 10 nm in diameter. In some embodiments, the apoptotic body surrogate is about 5 nm in diameter. In some embodiments, the apoptotic body surrogate is biodegradable. In some embodiments, the apoptotic body surrogate comprises a polyglycolic acid polymer (PGA), polylactic acid polymer (PLA), polysebacic acid polymer (PSA), poly(lactic-co-glycolic) acid polymer (PLGA), poly(lactic-co-sebacic) acid copolymer (PLSA), poly(glycolic-co-sebacic) acid copolymer (PGSA), polylactide co-glycolide (PLG), chitosan, or hyaluronic acid.

In some embodiments relating to various aspects, the condition is neuromyelitis optica.

In various aspects relating to methods described herein, the induction of tolerance may require a scavenger receptor. In some embodiments, the scavenger receptor comprises MARCO. In some embodiments, the induction of tolerance is sustained by a cytokine. In some embodiments, the cytokine is IL-10, IL-2 or TGF-β. In some embodiments, the apoptotic body surrogate is taken up by splenic cells expressing MARCO. In some embodiments, the composition is taken up by splenic cells expressing MARCO. In some embodiments, the composition is not taken up by splenic cells expressing SIGLEC-1. In some embodiments, the apoptotic body surrogate is not taken up by splenic cells expressing SIGLEC-1.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1J depict the role of the spleen and route of administration in Ag-SP tolerance induction. FIG. 1A, SJL/J mice were tolerized with 5×10⁷ sham OVA₃₂₃₋₃₃₁-SP given i.v. (OVA323-SP i.v.) or with 5×10⁷ PLP₁₃₉₋₁₅₁-SP given i.v. (PLP139-SP i.v.), s.c. (PLP139-SP s.c.), or i.p. (PLP139-SP i.p.). Five days later, the mice were immunized with 50 μg PLP₁₃₉₋₁₅₁/CFA and monitored for clinical EAE for 20 d postpriming. B-H, Scavenger receptor response to Ag-SP infusion. SJL/J mice were infused with 5×10⁷ PKH76-labled PLP₁₃₉-SP i.v., and spleens were harvested for immune-histochemical staining 3 h later. Expression of scavenger receptors LOX-1 (FIG. 1 B), SRBI (FIG. 1 C), and CD68 (FIG. 1 D) was unaffected, but SRBII was upregulated (FIG. 1 F) compared with noninfused mice (FIG. 1 E). Some colocalization of SRBII and PKH76 was observed (white arrowhead in FIG. 1 H), but not in isotype controls (FIG. 1 G). Scale bar, 50 μm. FIG. 1I and FIG. 1J, The spleen is required for Ag-SP tolerance. Sham-splenectomized (Sham Splx) or splenectomized (Splx) SJL mice were tolerized with OVA₃₂₃-SP or PLP₁₃₉-SP on day −7 and primed s.c. with 50 μg PLP₁₃₉₋₁₅₁/CFA on day 0, and DTH responses to PLP₁₃₉₋₁₅₁ were determined 8 d later (FIG. 1 I). Asterisks denote a significant reduction in DTH responses (*p<0.0005). Data are representative of four experiments of five mice per group. J, PLP₁₃₉₋₁₅₁-specific proliferative responses from Sham Splx and Splx SJL mice determined on day 10. Data examining the route of inoculation and splenectomized mice are representative of two or three experiments of five mice per group, with scavenger receptor examination determined from one experiment with five mice per group and at least five independent spleen sections examined. Asterisks denote a significant reduction in proliferative responses (*p<0.0001).

FIGS. 2A-2H depict rapid removal of intravenously infused Ag-SP from the spleen and the triggered IL-10 production thereby. FIG. 2A-2C, SJL/J mice were tolerized with 5×10⁷ PKH76-labeled OVA₃₂₃-SP. Groups of 3-5 mice were sacrificed at 0, 3, and 18 h postinfusion. At least 20, 8-μM section were examined from each animal. PKH76-labled subcellular debris present at 3 h (FIG. 2B) postinfusion was completely absent by 18 h (FIG. 2C). FIGS. 2D and 2E, A separate cohort of at least four animals was treated with 5×10⁷ CFSE-labeled OVA₃₂₃-SP, and mice were sacrificed 30 min and 3 h postinfusion. Numerous CFSE-labeled Ag-SP were observed at 30 min (FIG. 2D) but were completely absent by 3 h post0infusion (FIG. 2E). IL-10 is secreted in response to Ag-SP infusion. Groups of at least four mice were infused with 5×10⁷ OVA₃₂₃-SP; recipient spleens harvested at 0, 10, 60, and 180 min postinfusion; and IL-10 levels in supernatants of individual homogenized spleens (run in triplicate) were measured using ELISA. *IL-10 levels significantly higher than baseline (p<0.01). FIG. 2F, IL-10-deficient mice cannot be tolerized with OVA₃₂₃₋₃₃₉ (FIG. 2G). Wild-type (B6) and IL-10-deficient (IL-10gko) C57BL/6 mice were tolerized i.v. with 5×10⁷ syngeneic splenocytes from IL-10gko mice coupled with MOG₃₅₋₅₅ (irrelevant peptide control) or OVA₃₂₃₋₃₃₉ on day −5. On day 0, the mice were immunized with 200 μg OVA₃₂₃₋₃₃₉/CFA, and DTH responses to OVA₃₂₃₋₃₃₁ ear challenge were determined on day 7 (FIG. 2G). IL-10 neutralization prevents Ag-SP tolerance induction (FIG. 2H). Anti-IL-10 or control IgG2a Ab was given 30 min prior and 18 h after MOG₃₅-SP or OVA₃₂₃-SP infusion on day −5. Animals were immunized with OVA₃₂₃₋₃₃₉/CFA on day 0, and DTH was assessed on day 7. Data in all panels are representative of at least three experiments of at least four mice per group. Asterisks denote a significant reduction in DTH responses (*p<0.0005) as compared with MOG₃₅₋₅₅-SP controls.

FIGS. 3A-3E depict the lack of requirement for B cells for induction of Ag-SP tolerance. Wild-type (FIG. 3A) and B cell-deficient (pMT) C57BL/6 mice (FIG. 3B) were tolerized i.v. with 5×10⁷ syngeneic MOG₃₅-SP on day −7, primed with MOG₃₅₋₅₅/CFA on day 0, and monitored for clinical EAE disease for 24 d postpriming. Data are representative of two experiments of five mice per group. On day +25 postpriming, MOG₃₅₋₅₅-specific DTH responses were assessed (FIG. 3C). Wild-type SJL/J mice were treated with 250 μg control Ig (FIG. 3D) or anti-mouse CD20 mAb (clone 5D2) (FIG. 3E) on day −12, followed by i.v. tolerization with 5×10⁷ PLP₁₃₉-SP on day −7. Anti-CD20 treatment resulted in >95% reduction in B cells in the primary lymphoid organs, peritoncal cavity, and the blood within 2 d of Ab injection. On day 0, the mice were primed with PLP₁₃₉₋₁₅₁/CFA and monitored for disease incidence for 50 d postpriming. Data are representative of two experiments of five mice per group. Asterisks denote a significant reduction in mean clinical score or DTH responses (*p<0.01).

FIGS. 4A-4F depict the transferable nature of tolerance with CD4⁺ CD25⁺ T cells. SJL/J mice were tolerized on day −7 with 5×10⁷ OVA₃₂₃-SP or PLP₁₃₉-SP. On day 2, 5×10⁶ bulk splenocytes (SPL) or CD4⁺ splenocytes (SPL CD4⁺) from each treatment group were transferred i.v. to naive recipients that were primed s.c. with 50 μg PLP₁₃₉₋₁₅₁/CFA (FIG. 4A) or PLP₁₇₈₋₁₉₁/CFA (FIG. 4B) on day 0 and monitored for clinical disease. Asterisks denote a significant reduction in clinical score in recipients of bulk or CD4⁺ splenocytes (*p<0.05). Data are representative of two to three experiments of five to eight mice per group. FIG. 4C, Two mice from the groups receiving splenic CD4⁺ T cells from OVA₃₂₃-SP and PLP₁₃₉-SP primed with PLP₁₃₉₋₁₅₁/CFA were perfused on day +25. Spinal cords were stained with anti-CD4 (red) or anti-F4/80 (green) mAbs and counterstained with DAPI (blue). Lumbar regions are shown at original magnification ×200. FIG. 4D, Spleens were harvested from three representative mice from each group on day +25, and proliferative responses were determined. Data are representative of two experiments. E, SJL/J mice were tolerized on day −7 with OVA₃₂₃-SP or PLP₁₃₉-SP, as in A. On day −2, 5×10⁶ CD4¹ CD25- or CD4⁺CD25⁺ splenocytes from the tolerized mice were transferred i.v. to naive recipients that were primed s.c. with 50 μg PLP₁₃₉₋₁₅₁/CFA and monitored for clinical disease. Asterisks denote a significant reduction in clinical score in recipients of CD4⁺CD25⁺ splenocytes (*p<0.05) from PLP₁₃₉-SP-tolerized mice. Data are representative of two experiments of six to eight mice per group. FIG. 4F, SJL/J mice (5-6 per group) were treated with 500 μg control Ig (Cont. Ig) or anti-CD25 mAb (clone 7D4) on days −11 and −9, tolerized with 5×10⁷ OVA₃₂₃-SP or PLP₁₃₉-SP on day −7, primed with PLP₁₃₉₋₁₅₁/CFA on day 0, and monitored for clinical signs of disease. Data are representative of three separate experiments. Asterisks denote a significant reduction in clinical score of PLP₁₃₉-SP-treated mice (*p<0.01) in both control Ig and anti-CD25-treated mice.

FIGS. 5A-5F depict the dispensable nature of Tregs for tolerance induction by Ag-SP in contrast to the requirement of Tregs for long-term tolerance maintenance. FIG. 5A, SJL/J mice were treated with 500 μg control Ig (Cont. Ig) or anti-CD25 mAb (clone 7D4) on days −4 and −2. On day 0, the entire cohort of mice was tolerized with 5×10⁷ OVA₃₂₃-SP or PLP₁₃₉-SP. Separate groups of mice were primed with 50 μg PLP₁₃₉₋₁₅₁/CFA on day +14 (FIG. 5B), day +35 (FIG. 5C), or day +63 (FIG. 5D) posttolerization and followed for clinical signs of EAE. Data represent the clinical disease pattern of five to six mice per group and are representative of two separate experiments. FIGS. 5E and 5F, DTH responses of mice from C and Dto challenge with PLP₁₃₉₋₁₅₁ were determined following cessation of clinical disease assessment. Asterisks denote a significant reduction in clinical disease score (*p<0.01) and DTH responses (p<0.05).

FIGS. 6A-6S depict macrophage responses to Ag-SP in vivo and in vitro. In vivo response: Groups of at least five C57BL/6 mice were infused with nothing (No Ag-SP, FIGS. 6A, 6D, and 6G), 5×10⁷ non-ECDI-fixed PKH26 (red)-labeled splenocytes [PKH-SP (No ECDI), FIGS. 6B, 6E, and 6H], or 5×10⁷ ECDI-fixed PKH26-labeled MOG₃₅₋₅₅-SP (PKH Ag-SP, C, F, and I). Eight hours later, the spleens were harvested for immunohistochemistry. Spleen sections (8 μM) were stained in green for CD11c (FIG. 6A-C), F4/80 (FIG. 6D-F), and IL-10 (FIG. 6G-I), and counterstained with DAPI (blue, A-F). Similar to the nonfixed splenocyte control (6B), little colocalization of Ag-SP with CD11c was observed (6C). F4/80 commonly colocalized with PKH-26 in the Ag-SP-treated animals (6F). No IL-10 staining was observed in the untreated (6G) or non-ECDI-fixed splenocyte-infused animals (6H). Strong IL-10 production (indicated by the green stain) was commonly coincident with F4/80⁺ cells (indicated by the blue stain) (6I). In vitro response: The macrophage cell line, J774 (FIG. 6K-6M), thioglycolate-elicited (FIG. 6N-6P), and nonelicited peritoneal macrophages (FIG. 6Q-6S) were cultured on coverslips in 24-well plates and fed 10⁶ OVA₃₂₃₋₃₃₉-SP labeled with PKH26 (red) overnight. Supernatant was collected for IL-10 analysis, and the remaining coverslips were fixed in paraformaldehyde, counterstained with membrane dye PK1176 (green), and nuclei stained with DAPI (blue). Ag-SP remained PKH26⁺ after overnight incubation; the cells did not label with DAPI or PKH76 (FIG. 6J). J774 macrophages cultured alone (FIG. 6K) and demonstrated uptake of PKH26⁺ cell membranes (FIG. 6L), but failure to produce significant IL-10 (FIG. 6M). Thioglycolate-elicited peritoneal macrophages cultured alone (FIG. 6N) and demonstrated significant uptake of both fragments (white arrowhead) and cells (yellow arrowhead) (FIG. 6O), but failure to produce IL-10 (FIG. 6P). Resting peritoneal macrophages were cultured alone (FIG. 6Q) and demonstrated significant uptake of PKH26-labled Ag-SP (FIG. 6R) and significant production of IL-10 (FIG. 6S). Data represent at least six independent wells, conducted in two to three separate experiments. Asterisk represents significant increase in the level of IL-10 (p<0.05). Scale bars, 200 μm (FIG. 6A-6F), 50 μm FIG. 6G-6I).

FIGS. 7A-7J depict the splenic macrophages uptaking Ag-SP and expressing PD-L1 in an IL-10-dependent manner. Effect of Ag-SP infusion on splenic macrophage ration. Five groups of SJL/J mice (four to five mice per group) received IgG2a control Ab, anti-IL-10 alone, OVA₃₂₃₋₃₃₉-SP+IgG2a Ab, OVA₃₂₃₋₃₃₉-SP+anti-IL-10 Ab, or no treatment. All Abs were given 30 min prior to OVA₃₂₃-SP infusion. Three hours after infusion, animals were sacrificed and splenocytes stained with a mixture of Abs, as described in the Examples described herein. FIG. 7A, Splenic APC populations were enumerated using the gating strategy shown; black population indicates the ungated isotype control for each dot plot. FIG. 7B, Percentages of CD4⁺ DCs, CD8α⁺ DCs, and plasmacytoid DCs did not change in any of the treatment groups, but percentages of macrophages increased in an IL-10-dependent fashion. F4/80⁺ splenic macrophages uptake Ag-SP and express PDL-1. Spleens from CD45.1 C57BL/6 mice receiving either PBS (FIG. 7C) or 5×10⁷ CD45.2⁺ PKH-26-labeled OVA₃₂₃-SP (FIG. 7D) were harvested 2.5 h after i.v. administration. Gate R1 represents recipient cells that have taken up donor Ag-SP, whereas gate R2 represents intact Ag-SP. Numbers adjacent to gate represent the percentage of cells within the gate (FIG. 7D). Relative CD45.2 expression on gates R1 (gray line) and R2 (black line) (FIG. 7E). Cells from gate R1 are 85% CD11b⁺ and 11.6% CD11c^{high} (FIG. 7F). Cells from gate R3 are 77.5% F4/80^{int} and 11.3% F4/80^{high}. The majority of the cells in gate R3 were CD11cint, which is consistent with the phenotype of splenic MZ macrophages (FIG. 7G). Greater than 73% of cells from R3 (i.e., those that are of recipient origin, the majority being F4/80⁺ macrophages) that have engulfed AG-SP express PD-L1 (FIG. 7H). PD-L1 expression increases in the CD8α⁺ DC and F4/80⁺ macrophage populations, and expression is reversed by anti-IL-10 in macrophages (FIG. 7). FIG. 7J, DTH and change in ear swelling in anti-PD-L1-treated animals at 7 d postpriming. Data are representative of two separate experiments. Asterisks denote a significant change in APC subset ratio/expression compared with animals treated with IgG2a control AB (*p<0.05). PD-L1 expression is required for Ag-SP tolerance. PD-L1 blockade prevents AG-SP tolerance induction. SJL/J mice were treated with anti-PD-L1 or control IgG2a Ab, as detailed in the Examples described herein. Mice were tolerized with OVA₃₂₃-SP or PLP₁₃₉-SP on day −7. Animals were immunized with PLP₁₃₉₋₁₅₁/CFA in day 0, and DTH was accessed on day 7. Results are representative of two separate experiments of at least five mice per group. Asterisks denote a significant reduction in DTH responses (*p<0.01) as compared with MOG₃₅₋₅₅-SP controls.

FIG. 8 depicts examples for microspheres encapsulating regulatory cytokines and microspheres tagged with apoptotic flags.

FIGS. 9A-9C depict the effect of administration of peptide-coupled polystyrene microspheres either prior to, or after induction of PLP₁₃₉₋₁₅₁ induced EAE in mice. (FIG. 9A) Pre-treatment with peptide-coupled microspheres prior to priming with PLP₁₃₉₋₁₅₁+Complete Freund's Adjuvant (CFA); (FIG. 9B) Pre-treatment with peptide-coupled microspheres prior to priming with PLP₁₇₈₋₁₉₁+Complete Freund's Adjuvant (CFA); (FIG. 9C) Post-treatment with peptide-coupled microspheres following priming with PLP₁₃₉₋₁₅₁+Complete Freund's Adjuvant (CFA).

FIGS. 10A-10B depict route and size Requirements for tolerance induction using peptide-coupled Polystyrene microbeads. PLP₁₃₉₋₁₅₁ or a control (OVA₃₂₃₋₃₃₉) peptide was ECDI-coupled to 0.1, 0.5, 0.75 or 4.5 μm polystyrene microspheres. An ECDI-free (NO ECDI) bead mixture was prepared omitting ECDI coupling. Mice were injected intravenously or subcutaneously with either the PLP₁₃₉₋₁₅₁ or control (OVA₃₂₃₋₃₃₉) peptide bound or ECDI-free microspheres on day 0 relative to priming with PLP₁₃₉₋₁₅₁.

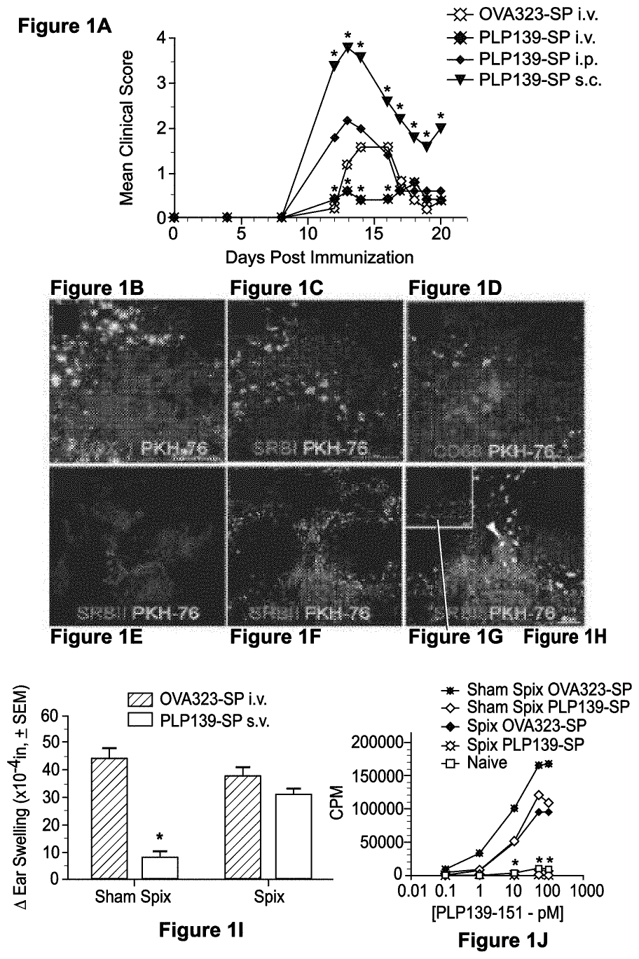

FIG. 1I depicts the requirement for the MARCO scavenger receptor for tolerance induction using peptide-coupled polystyrene microbeads, but not peptide-coupled SP. Wild-type BALB/c and MARCO knockout mice were tolerized with ECDI-coupled polystyrene microspheres with MOG₃₅ peptide (MOG₃₅-MP), with OVA₃₂₃₋₃₃₉ peptide (OVA₃₂₃₋₃₃₉-MP), or ECDI-coupled splenocytes with OVA₃₂₃₋₃₃₉ peptide (OVA₃₂₃₋₃₃₉-SP). Subsequently, mice were primed with OVA₃₂₃₋₃₃₉ and CFA. Control mice were not tolerized or immunized (naive). Mice were observed for ear swelling as a measure of immune response.

FIGS. 12A-12C depict effective downregulation of induction and progression of PLP₁₃₉₋₁₅₁ R-EAE with PLP₁₃₉₋₁₅₁-coupled polystyrene and PLG microbeads. Three groups of five R-EAE mice were tolerized with ECDI-coupled polystyrene microspheres with PLP₁₃₉₋₁₅₁, ECDI-coupled PLG microspheres with PLP₁₃₉₋₁₅₁, or with PLG alone on day −7 and primed on day 0. Mean clinical scores are displayed on a daily basis (FIG. 12A) and in a cumulative fashion (FIG. 12B). Ear swelling is displayed for each of the three groups (FIG. 12C).

FIG. 13A-13B depict the localization of PLG (FIG. 13A) and polystyrene (FIG. 13B) microbeads to the marginal zone of the spleen.

FIGS. 14A-14L depict antigen-coupled polystyrene microparticles as effective tools for inducing tolerance for the prevention and treatment of EAE. Antigen-coupled polystyrene microparticles are effective for inducing tolerance for the prevention and treatment of EAE. (FIG. 14A) Mean clinical score of SJL/J mice injected i.v. with 500-nm carboxylated PSB coupled to PLP₁₃₉₋₁₅₁ (PLP139-PSB) or OVA₃₂₃₋₃₃₉ (OVA323-PSB) 7 d before initiation of EAE by s.c. immunization with PLP₁₃₉₋₁₅₁ plus CFA. A separate group was tolerized with PLP₁₃₉₋₁₅₁-SP (PLP139-SP). No Rx, no treatment. (FIG. 14B, 14C) Mean clinical score of mice that received PLP₁₃₉₋₁₅₁-PSB, OVA₃₂₃₋₃₃₉-PSB or unconjugated PSB at the onset of hindlimb paralysis (11 d after priming); disease symptoms were scored for a total of 35 and 66 d, respectively. (FIG. 14D) Mean clinical score of mice injected i.v. with 500-nm carboxylated PSB coupled to PLP₁₃₉₋₁₅₁, OVA₃₂₃₋₃₃₉ or nothing 7 d before induction of EAE with PLP₁₃₉₋₁₅₁. (FIG. 14E, 14F) Ear swelling, as a measure of DTH, 24 h after ear challenge with the priming PLP₁₃₉₋₁₅₁ epitope (FIG. 14E) or the PLP₁₃₉₋₁₅₁ spread epitope (FIG. 14F) at 36 d after priming in selected representative mice from the PLP₁₃₉₋₁₅₁ plus CFA (PLP139′CFA)-primed groups in a (OVA₃₂₃₋₃₃₉-PSB, PLP₁₃₉₋₁₅₁-PSB and no particles). Additional mice included in this analysis received doses of PSB i.v. but were not primed for EAE. Responses to a control OVA₃₂₃₋₃₃₉ peptide were subtracted from each measure of ear swelling. (FIGS. 14G,14H) The number of CD45^{hi} cells (FIG. 14G) and CD3⁺CD4⁻¹ T cells (FIG. 14H) determined by flow cytometry at the onset of disease (day 12), peak of disease (day 14) and remission (day 20) in the brains and spinal cords of SJL/J mice injected i.v. with 500-nm carboxylated PSB coupled with PLP₁₃₉₋₁₅₁, OVA₃₂₃₋₃₃₉ Or nothing 7 d before EAE priming with PLP₁₃₉₋₁₅₁ plus CFA. (FIG. 14I) Mean clinical score in SJL/J mice treated with i.v. injection of 500-nm carboxylated PSB incubated with PLP₁₃₉₋₁₅₁ in the presence or absence of ECDI or treated with 500-nm OVA₃₂₃₋₃₃₉-PSB 7 d before priming with PLP₁₃₉₋₁₅₁ plus CFA. (FIG. 14J) Mean clinical score in SJL/J mice treated i.v. with PSB of varying diameters (100 nm, 500 nm, 1.75 μm or 4.5 μm) coupled to PLP₁₃₉₋₁₅₁ or treated with 500-nm OVA₃₂₃₋₃₃₉-PSB 7 d before priming with PLP₁₃₉₋₁₅₁ plus CFA. (FIG. 14K) Mean clinical score in SJL/J mice treated with 500-nM PLP₁₃₉₋₁₅₁-PSB or OVA₃₂₃₋₃₃₉-PSB in the lateral tail vein (i.v.) or on the flank (s.c.) 7 d before priming with PLP₁₃₉₋₁₅₁ plus CFA. (FIG. 14L) In vitro proliferative responses to stimulation with the PLP₁₃₉₋₁₅₁ priming epitope or a control peptide (OVA₃₂₃₋₃₃₉) determined by [³H]-thymidine uptake in spleens and lymph nodes collected from a subset of the mice in k. CPM, counts per minute. All experiments consisted of 5-10 mice per group and are representative of 2-4 repeats. *P≤0.05 (ANOVA₃₂₃₋₃₃₉) for the differences in mean clinical scores, DTH responses, numbers of CNS-infiltrating cells or proliferative responses compared to the responses in group tolerized to the appropriate irrelevant control peptide. Error bars, s.e.m.

FIGS. 15A-15I depict Ag-PSB localization in MARCO, SIGN-R1′ splenic marginal zone macrophages (MZM) and the requirement for MARCO for Ag-PSB tolerance induction. MARCO has a crucial role in tolerance induction using antigen-coupled microparticles. (FIG. 15A-15F) MARCO (FIG. 15A, 15D, red), SIGN-R1 (FIG. 15B, 15E, red), SIGLEC-1 (FIG. 15C, 15F, red) and 4,6-diamidino-2-phenylindole (DAPI, blue) staining in dissected and snap-frozen spleens from mice infused with PSB (no PSB) or FITC-labeled MOG-PSB (MOG35-PSP, green). Arrowheads indicate phagocytized PSB. (FIG. 15G) Ear swelling 24 h after ear challenge with OVA₃₂₃₋₃₃₉ or an irrelevant peptide (PLP₁₃₉₋₁₅₁) in WT or Marco−/− BALB/c mice injected i.v. with OVA₃₂₃₋₃₃₉-PSB (OVA323-PSB) or control MBP84-104-PSB (MBP84-PSB) 7 d before immunization with OVA₃₂₃₋₃₃₉ plus CFA. Ear challenge, as a measure of DTH, was performed 8 d after immunization. (FIG. 15H, 15I) Ear swelling as described for g in WT (FIG. 15I) and Marco−/− (FIG. 15H, 15I) BALB/c mice treated i.v. with OVA₃₂₃₋₃₃₉-PSB, soluble OVA₃₂₃₋₃₃₉ (sol. OVA323) (FIG. 15H), MOG-PSB (FIG. 15I) or OVA₃₂₃₋₃₃₉-SP (FIG. 15I) 7-8 d before immunization with OVA₃₂₃₋₃₃₉ plus CFA. All experiments consisted of 5-10 mice per group and are representative of at least 2-4 separate experiments. *P≤0.05 (ANOVA₃₂₃₋₃₃₉) for differences in mean clinical scores and DTH responses compared to the responses in groups tolerized to the appropriate irrelevant control peptide, Error bars, s.e.m.

FIGS. 16A-16E depict response of antigen-specific T cells to tolerance induction with Ag-PSB. Response of antigen-specific T cells to tolerance induction with Ag-PSB. (FIG. 16A) T Cell content 48, 39 and 168 h after treatment in female DO11.10 OVA₃₂₃₋₃₃₉-specifc TCR transgenic mice treated i.v. with 500-nm carboxylated PSB coupled to the cognate peptide (OVA₃₂₃₋₃₃₉, OVA323-PSB) or an irrelevant peptide (MBP₈₅₋₉₉, MBP85-PSB). Proliferation, measured by [³H]-thymidine uptake, in 3×10⁵ T cells magnetically purified from peripheral blood (FIG. 16B) and peripheral lymph nodes (FIG. 16C) of DO11.10 mice treated i.v. with 500-nm carboxylated PSB coupled to the cognate peptide (OVA₃₂₃₋₃₃₉) or an irrelevant peptide (MOG₃₅₋₅₅, MOG35-PSB) and re-stimulated 48 h later with 1 mg ml-¹ cognate OVA₃₂₃₋₃₃₉ peptide or PLP₁₃₉₋₁₅₁ in vitro. Mean clinical score in naive SJL/J mice treated with control immunoglobulin in (Cont. Ig) or anti-IL-10 (JES5-16E3; 200 μg intraperitoneally (i.p.)) (FIG. 16D) or control immunoglobulin in or anti-CD25 (PC61; 500 μg i.p.) (FIG. 16E) 1 d before and 1 d after treatment with either OVA₃₂₃₋₃₃₉-PSB or PLP₁₃₉₋₁₅₁-PSB (PLP139-PSB); 7 d after tolerization, mice were primed for EAE with PLP₁₃₉₋₁₅₁ plus CFA. Data are representative of three separate experiments. Error bars, s.e.m. *P≤0.05 (Student's t test) for the differences in T cell numbers, CPM and mean clinical scores compared to the responses in groups tolerized to the appropriate irrelevant control peptide.

FIGS. 17A-17C depict the effect of Ag-PSB on antigen-specific T-cells in female DO11.10 mice after i.v. treatments of 0.5 μm carboxylated PSBs coupled to cognate antigen (OVA₃₂₃₋₃₃₉) or irrelevant antigen (MBP₈₅₋₉₉). Female D011.10 mice were given i.v. treatments of 500 nm carboxylated PSBs coupled to cognate antigen (OVA323-339) or irrelevant antigen (MBP85-99). (FIG. 17A) Peripheral blood was analyzed for T cell content at 1, 24, and 48 h post-treatment. At 48 h, spleens, lymph nodes and peripheral blood from select mice were collected and OVA323-339-specific T cell content was quantified by flow cytometry using the DO11.10 TCR-specific antibody KJ-126. The total numbers of CD4⁺KJ-126⁺ cells (FIG. 17B) and the numbers of CD4⁺KJ-126⁺ cells/ml of peripheral blood (FIG. 17C) are shown. Results are representative of 3 separate experiments. *Numbers of KJ-126⁺ T cells in the peripheral blood of OVA323-PSB treated mice is significantly less than those in the MBP85-99-PSB (p<0.001, Student's t-test). Error bars indicate standard error of the mean.

FIGS. 18A-18B depict antigen-specific T cells undergoing suboptimal proliferation in response to Ag-PSB. Antigen-specific T cells undergo suboptimal proliferation in response to Ag-PSB. (FIG. 18A) Results from naive SJL/J (CD90.2⁺) recipient mice exposed i.v. to PLP₁₃₉₋₁₅₁-PSB (i, ii) s.c. to PLP₁₃₉₋₁₅₁ plus CFA (iii, iv) or i.v. to OVA₃₂₃₋₃₃₉-PSB (v, vi) 48 h after being transferred with naive CD90.1⁺ PLP₁₃₉₋₁₅₁-specific 5B6 TCR transgenic T cells sorted from donor lymph nodes and labeled with CFSE. Five days after these treatments (Pi.), spleens and lymph nodes (LNs) were collected, and the percentage of diving CD90.1⁺ T cells was assessed by measuring CFSE dilution using flow cytometry. (FIG. 18B) Flow cytometric analyses of CFSE dilution at 5 d after priming in mice additionally treated with PLP₁₃₉₋₁₅₁-PSB (i, ii) or OVA₃₂₃₋₃₃₉-PSB (iii, iv) 5 d after the initial treatments in a and then primed with PLP₁₃₉₋₁₅₁ plus CFA. Three separate mice were analyzed in each group with representative plots shown. Data shown are representative of three separate experiments. Percentages in graphs reflect the percent of T cells that have divided.

FIGS. 19A-19D depict activation of naive T cells to direct Ag-PSB engagement and cytokine responses of Ag-PSB tolerized T cells to peptide immunization. Antigen-specific T cells are abortively activated after Ag-PSB encounter but do not synthesize IL-17A and I FN-γ after direct in vivo exposure to Ag-PSB or after subsequent immunogenic stimulation. (FIG. 19A, 19B) Flow cytometric analysis of T-cell activation markers CD62L, CD69 and CD44 5 d after treatment in spleens and lymph nodes (LNs) from naive CD90.2-SJL/J recipients after being transferred with CFSE-labeled naive CD90.1⁺ 5B6 TCR transgenic T cells and then treated i.v. with PLP₁₃₉₋₁₅₁-PSB (PLP139-PSB) or primed s.c. with PLP₁₃₉₋₁₅₁ plus CFA. Transgenic T cells were identified by CD90.1 and CFSE signals. (FIG. 19C, 19D) Naive CD90.1⁺ 5B6 TCR transgenic T cells were CFSE labeled and transferred to naive CD90.2⁺ SJL/J recipients that were then treated i.v. with PLP₁₃₉₋₁₅₁-PSB or primed s.c. with PLP₁₃₉₋₁₅₁ plus CFA. Where indicated, SJL/J recipients of naive CFSE-labeled 5B6 TCR transgenic T cells were treated i.v. with OVA₃₂₃₋₃₃₉-PSB or PLP₁₃₉₋₁₅₁-PSB and primed with PLP₁₃₉₋₁₅₁ plus CFA 5 d later. Data shown are representative of three independent experiments.

FIGS. 20A-20K depict the role of anergy induction on short-term tolerance induced by i.v. treatment with Ag-PSB. Short-term tolerance induced by i.v. treatment with Ag-PSB is caused primarily by anergy induction. SJL/J mice were treated i.v. with OVA₃₂₃₋₃₃₉-PSB (OVA323-PSB) or PLP₁₃₉₋₁₅₁-PSB (PLP139-PSB) 7 d before s.c. priming with PLP₁₃₉₋₁₅₁ plus CFA. Eight days after priming, the proliferation of spleen and lymph nodes in response to stimulation with the priming antigen (PLP₁₃₉₋₁₅₁) or a control antigen (OVA₃₂₃₋₃₃₉) in the presence of absence of 200 U ml⁻¹ of exogenous IL-2 was measured (FIG. 20A). Supernatants were collected for measurements of secreted IFN-γ (FIG. 20B) and IL-17A (FIG. 20C). Results are representative of 3-4 separate experiments. SJL/J mice were treated with 500-nm FITC-PSB (FIG. 20D) or 500-nm biodegradable FITC-PLG microparticles (FIG. 20E) coupled with PLP₁₃₉₋₁₅₁. Twelve hours later, frozen spleen sections were prepared from a subset of mice, and these sections were counterstained with DAPI (blue). On day −7 (FIGS. 20F-20H) or day −11 (FIG. 20) relative to PLP₁₃₉₋₁₅₁ plus CFA priming, mice were injected with 500-nm PLP₁₃₉₋₁₅₁-PSB or PLP₁₃₉₋₁₅₁-PLG (PLP139-PLG) microparticles and monitored for development of clinical disease by assessing mean clinical score (FIGS. 20F, 20I) and cumulative mean clinical score (FIG. 20G) over time. (FIG. 20H) At the conclusion of the experiment, the mice from f were ear challenged with PLP₁₃₉₋₁₅₁, and DTH responses were determined. SJL/J mice were tolerized with 500-nm PLP₁₃₉₋₁₅₁-PLG (PLP178-PLG) or OVA₃₂₃₋₃₃₉-PLG (OVA₃₂₃₋₃₃₉323-PLG) microparticles on day +25 after PLP₁₃₉₋₁₅₁ plus CFA priming (FIG. 20J) or with 500-nm PLP₁₃₉₋₁₅₁-PLG or OVA₃₂₃₋₃₃₉-PLG microparticles on day +18 after PLP₁₃₉₋₁₅₁ plus CFA priming (FIG. 20K) and monitored for clinical disease. Error bars, s.e.m. *P≤0.01 (ANOVA) for the differences in proliferation, mean clinical scores and DTH responses compared to groups tolerized to sham PLG particles. Data shown are representative of 2-3 separate experiments of 5-7 mice per group.

### DETAILED DESCRIPTION

The present invention provides compositions and methods for inducing antigen-specific tolerance in a subject. In one embodiment, the present invention provides a composition comprising an apoptotic body or apoptotic body surrogate, and an epitope of an antigen. Also provided herein are methods of preparing and administering the composition. The composition and methods provided herein can induce antigen-specific tolerance in a subject.

With aberrant T-cell activation underlying many autoimmune disorders, solutions comprising induction T-cell tolerance are critical for treating these diseases. According to the methods and compositions of the invention, mimicking strategies for tolerance induction that exploit natural mechanisms for clearing apoptotic debris, antigen-decorated microparticles (˜500-nm diameter) are capable of inducing long-term T-cell tolerance in mice with relapsing experimental autoimmune encephalomyelitis. Specifically, intravenous infusion of either polystyrene or biodegradable poly(lactide-co-glycolide) microparticles bearing encephalitogenic peptides prevents the onset and modifies the course of the disease. These beneficial effects are associated with microparticle uptake by marginal zone macrophages expressing the scavenger receptor MARCO and are mediated in part by the activity of regulatory T cells, abortive T-cell activation and T-cell anergy. Together the data herein highlight the potential for using microparticles to target natural apoptotic clearance pathways to inactivate pathogenic T cells and halt the disease process in autoimmunity.

Intravenous administration of soluble peptides crosslinked to syngeneic splenic leukocytes using ethylene carbodiimide (ECDI) safely and efficiently induces antigen-specific immune tolerance, is effective in the prevention and treatment of T helper type 1 (TH1) cell- and/or TH17 cell-mediated autoimmune diseases and overcomes many of the drawbacks of the failed trials involving monoclonal antibodies and soluble peptides. However, the challenge of isolating isologous leukocytes and peptide coupling under good manufacturing practices (GMP) may inhibit clinical application of this therapy. The mechanism underpinning the beneficial effect of this strategy involves the delivery of an antigen in the context of apoptotic carrier cells. Therefore, methods and compositions described herein seek to achieve similar results using microparticles, e.g. 500-nm diameter, mimicking apoptotic cells and/or cell debris. In many applications, inert microparticles are used for this task.

Methods and compositions described herein, comprising microparticles coupled to encephalitogenic myelin epitopes prevent and treat the clinical symptoms of experimental autoimmune encephalomyelitis (EAE), a mouse model of multiple sclerosis. This treatment reduces inflammatory cell infiltration into and damage of the central nervous system (CNS). The beneficial effect of some microparticles is associated with the scavenger receptor MARCO, as mice deficient in MARCO are resistant to tolerance induced by these antigen-linked microparticles but not by soluble peptide or antigen-coupled apoptotic cells. Furthermore, tolerance induced by peptide-coupled microparticles may depend on the induction of T-cell anergy and/or the activity of regulatory T (T_{reg}) cells.

According to methods and compositions of the invention, it is understood that antigenic peptides coupled to splenic leukocytes can be used as treatments in preclinical models of autoimmune disease, allergy and transplantation. In some embodiments, inert microparticles can be used as surrogates for apoptotic leukocytes as antigen ‘carriers’. Inert microparticles coupled to peptides, can be produced in large amounts under GMP conditions. Polystyrene and biodegradable PLG micro-particles can be highly efficient substitutes for apoptotic cells. These can be taken up in a MARCO scavenger receptor-dependent fashion and are capable of inducing long-term antigen-specific T-cell abortive activation and/or anergy.

In various embodiments of the invention, antigenic peptides are covalently linked to microparticles, e.g. about 500-nm. Intravenous (i.v.) administration can be chosen and appears to deliver the antigen-linked particles to the splenic marginal zone for efficient tolerance induction. Without being bound by theory, it is understood that compared to 20-nm diameter particles, 200-nm and 1,000-nm diameter particles have a higher propensity to bind to MARCO receptors in vitro. Data described herein show that MARCO-expressing MZM, but not SIGLEC-1-expressing metallophilic macrophages, take up peptide-linked particles, ascribing a novel role to MARCO in T-cell tolerance. Without wishing to be bound by any theory, MARCO appears to function through its ability to take up antigen-linked particles and assist in macrophage antigen presentation and/or antigen transfer to local dendritic cells. MARCO may also inhibit inflammatory responses by preventing dendritic cell migration or by other unknown anti-inflammatory mechanisms. Data described herein show that, while macrophage production of IL-10 is thought to be crucial for tolerance to apoptotic cells, IL-10 neutralization failed to completely inhibit the tolerance induced by antigenic peptides coupled to microparticles. The MARCO pathway of tolerance induction may be specific for microparticle-bound peptide, as Marco−/− mice were effectively tolerized to soluble peptide and peptides coupled to apoptotic splenic leukocytes.

Clinical translation of tolerance-based therapies for the treatment of autoimmune disease may be established through the ability to suppress pre-existing autoreactive effector T cells and/or establish tolerance of naive autoreactive T cells that may be activated after exposure to endogenous autoantigens released from damaged target organs (epitope spreading). In various embodiments, methods and compositions of the invention directed to R-EAE and the disorders represented thereby, such as multiple sclerosis or acute disseminated encephalomyelitis, are effective in prophylactically preventing the disorders, inhibiting established disorders and suppressing relapse caused by epitope spreading. As described herein, the tolerizing effects of the invention can be realized through i.v. administration of microparticle linked antigenic molecules, such as peptides or proteins.

The methods and compositions of the invention, thus support the use of antigen-coupled microparticles as a tool for tolerance induction. This application has broad therapeutic utility in various immune and auto-immune conditions, such as airway allergy and allotolerance.

A composition for induction of antigen-specific tolerance in a subject suffering from or at risk of a condition is provided. The composition can induce tolerance to one or more antigens in the subject, in which the antigen would otherwise act as an allergen that induces T-cell receptor-mediated stimulation in the subject (such as if the subject was not administered the composition). The composition can comprise an apoptotic body surrogate and one or more epitopes. For example, the epitope can be an immunodominant epitope. In one embodiment, the composition comprises an apoptotic body surrogate and a plurality of immunodominant epitopes. The one or more immunodominant epitopes can be associated with one or more antigens suspected to cause a condition in a subject. The composition can further comprise an additional anergy promoting agent.

Also provided herein is a method of administering a composition comprising an apoptotic body surrogate and one or more epitopes, wherein tolerance to at least one or more antigens is induced specifically in the subject. The epitope can be an immunodominant epitope. In one embodiment, the composition comprises an apoptotic body surrogate and a plurality of immunodominant epitopes. The one or more immunodominant epitopes can be associated with one or more antigens suspected to cause a condition in a subject. The method can further comprise administering an additional anergy promoting agent.

The compositions and method disclosed herein can be used to reduce a hypersensitivity response in a subject, such as a subject's hypersensitivity to a food allergy or therapeutic. In one embodiment, a hypersensitivity response to a food allergy is reduced in a subject. The method can comprise administering a composition comprising an apoptotic body surrogate and an immunodominant epitope of a food to the subject, wherein the composition induces tolerance to the food in the subject thereby reducing the hypersensitivity response of the food allergy in the subject. In another embodiment, a hypersensitivity response to a therapeutic in a subject is reduced by administering a composition comprising an apoptotic body surrogate and an epitope of a therapeutic, wherein the composition induces tolerance of the therapeutic in the subject.

Also provided herein is a method of reducing the risk of transplant rejection in a subject. The method can comprise administering a composition comprising an apoptotic body surrogate and an immunodominant epitope of a tissue to be transplanted to said subject, wherein the composition induces tolerance of the tissue that is transplanted or to be transplanted in the subject, thereby reducing the risk of transplant rejection in the subject.

A method of inducing antigen-specific tolerance in a subject from or at risk of hypersensitivity to a antigen can also comprise obtaining personalized information of a subject and determining from the personalized information an antigen to which the subject is hypersensitive to. The method can further comprise administering a composition comprising an apoptotic body or apoptotic body surrogate and an epitope of the antigen to the subject, thereby inducing tolerance specific to said antigen in said subject.

A method of inducing antigen-specific tolerance in a subject suffering from or at risk of hypersensitivity to an antigen can also comprise obtaining a pool of immune cells from a subject and determining from the pool an antigen to which the subject is hypersensitive to. The method can further comprised administering a composition comprising an apoptotic body or apoptotic body surrogate and an epitope of the antigen to the subject, thereby inducing tolerance specific to said antigen in said subject.

Also provided herein is a method of delivering an antigen to a splenic marginal zone of a subject comprising administering a composition comprising an apoptotic body surrogate and an antigen to a subject. The apoptotic body surrogate can be recognized by a macrophage scavenger receptor and the macrophage scavenger receptor can uptake and deliver the apoptotic body surrogate, antigen, or both to the splenic marginal zone.

The disclosed compositions and methods can be effective in inducing antigen-specific tolerance and/or prevent the onset of an immune related disease and/or diminish the severity of a pre-existing immune related disease. In some embodiments, the compositions and methods of the present invention can cause T cells to undertake early events associated with T-cell activation, but do not allow T-cells to acquire effector function, For example, administration of compositions of the present invention can result in T-cells having a quasi-activated phenotype, such as CD69 and/or CD44 upregulation, but do not display effector function, such as indicated by a lack of IFN-γ or IL-17 synthesis. In some embodiments, administration of compositions of the present invention results in T-cells having a quasi-activated phenotype without having conversion of naive antigen-specific T-cells to a regulatory phenotype, such as those having CD25⁺/Foxp3⁺ phenotypes.

Various aspects of the present invention are described in further detail herein.

### Definitions Science

As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages. Immune cells involved in the immune response include lymphocytes, such as B cells and T cells (CD4⁺, CD8⁺, TH1 and TH2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

As used herein, the term “anergy,” “tolerance,” or “antigen-specific tolerance” refers to insensitivity of T cells to T cell receptor-mediated stimulation. Such insensitivity is generally antigen-specific and persists after exposure to the antigenic peptide has ceases. For example, anergy in T cells is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and subsequently failure to proliferate. Generally, a failure to produce cytokines prevents proliferation. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al., 1992 Science, 257:1134).

As used herein, the term “immunological tolerance” refers to methods performed on a proportion of treated subjects in comparison with untreated subjects where: a) a decreased level of a specific immunological response (thought to be mediated at least in part by antigen-specific effector T lymphocytes, B lymphocytes, antibody, or their equivalents); b) a delay in the onset of progression of a specific immunological response; or c) a reduced risk of the onset or progression of a specific immunological response. “Specific” immunological tolerance occurs when immunological tolerance is preferentially invoked against certain antigens in comparison with others.

### Apoptotic Body and Apoptotic Body Surrogate

The present invention provides compositions and methods for inducing antigen-specific tolerance in a subject comprising an apoptotic body, or apoptotic body surrogate, and an epitope of an antigen.

Apoptosis is the process of programmed cell death during which an apoptotic body is produced from a cell undergoing apoptosis. Biochemical events lead to characteristic cell changes or morphological changes, and death of cells in apoptosis. These changes can include blebbing, cell shrinkage, nuclear fragmentation, chromatic condensation, and chromosomal DNA fragmentation. In contrast to necrosis, apoptosis produces cell fragments called apoptotic bodies that phagocytic cells are able to engulf. Phagocytosis is believed to allow quick removal of dead cells, before the contents of the cell can spill out onto surrounding cells and cause damage.

Thus, in one embodiment, the composition and method disclosed herein comprises an apoptotic body derived from an apoptotic cell. In another embodiment, a composition comprises an apoptotic cell, such that when administered to a subject, one or more apoptotic bodies are formed or derived from the apoptotic cell. An apoptotic cell or apoptotic body can be generated for use in a composition or method disclosed herein. For example, ethylene carbodiimide (ECDI) conjugation to a cell can cause apoptosis of the cell. ECDI-conjugated cells, such as cells conjugated to one or more antigens or epitopes, can be used in one or more compositions and methods disclosed herein. Candidate reagents or methods can be screened using an assay for apoptosis to select a reagent or method to generate an apoptotic body or apoptotic body surrogate.

Many commercial assays are available to detect apoptosis, such as assays to detect caspase activity, as caspases are activated during apoptosis. The assay can comprise detecting activation of the caspase, such as detecting zymogen processing of the one or more caspases, or detection of caspase function. Examples of such assays include, but are not limited to, PhiPhiLux® (Oncolmmunin, Inc.), Caspase 3 Activity Assay (Roche), Homogeneous Caspases Assay (Roche Applied Science), Caspase-Glo™ Assays (Promega), Apo-ONE® Homogeneous Caspase-3/7 Assay (Promega), CaspACE™ Assay System, Colorimetric (Promega), CaspACE™ Assay System, Fluorometric (Promega), EnzChek® Caspase-3 Assay Kit #1 (Invitrogen), Image-iT™ LIVE Green Caspase-3 and -7 Detection Kit (Invitrogen), Active Caspase-3 Detection Kits (Stratagene), Caspase-mediated Apoptosis Products (BioVision), and CasPASE™ Apoptosis Assay Kit (Genotech).

During fragmentation of the nucleus, endonuclease activation leaves short DNA fragments, which are often regularly spaced in size. These give a characteristic “laddered” appearance in electrophoresis, and this DNA laddering can be used to identify apoptosis. Many commercial assays available for detecting apoptosis is based on detecting this DNA fragmentation, such as with terminal deoxynucleotidyl transferase dUTP nick end labeling (TUNEL) or other types of DNA fragmentation assays. Examples of such assays include, but are not limited to, Apoptotic DNA Ladder Kit (Roche), Cellular DNA Fragmentation ELISA (Roche), Cell Death Detection ELISAPLUS (Roche), In Situ Cell Death Detection Kit (Roche), DeadEnd™ Fluorometric TUNEL System (Promega), DeadEnd™ Colorimetric TUNEL System (Promega), APO-BrdU™ TUNEL Assay Kit (Invitrogen), TUNEL Apoptosis Detection Kit (Upstate), Apoptosis Mebstain Kit (Beckman Coulter), Nuclear-mediated Apoptosis Kits (BioVision), and Apoptotic DNA Ladder Kit (Genotech).

Another assay for apoptosis is the assay for detecting annexin V. Annexin V binds to phosphatidylserine (PS). Dying cells that undergo the final stages of apoptosis display phagocytotic molecules, such as PS on their cell surface. PS is normally found on the cytosolic surface of the plasma membrane, but is redistributed during apoptosis to the extracellular surface by a hypothetical protein. This allows PS to be indirectly detected by annexin V staining. Such commercially available assays include, but are not limited to, Annexin V, Alexa Fluor® 350 conjugate (Invitrogen), Rhodamine 110, bis-(L-aspartic acid amide), trifluoroacetic acid salt (Invitrogen), Annexin V, Alexa Fluor® 488 (Cambrex), and Annexin V Apoptosis Kits (BioVision).

Other assays for apoptosis can be for detecting of apoptotic markers, such as for Poly(ADP-ribose) polymerase (PARP), which is a nuclear enzyme involved in DNA repair. In many cell types, an early event during apoptosis is the proteolytic cleavage of PARP by a caspase. Thus, detecting of PARP, such as with anti-PARP, such as commercially available antibodies including, but not limited to anti-PARP from Roche, can be used for Western blot detection of the resulting proteolytic PARP fragments in extracts from early apoptotic cells. Another example is the detecting of cytokeratins. Cytokeratins, in particular cytokeratin 18, are subjected to proteolytic cleavage during the early stages of apoptosis. An antibody to detect one or more cytokeratins, such as the monoclonal antibody M30 CytoDEATH, which recognizes a specific caspase-cleavage site within cytokeratin 18 that is not detectable in the native CK18 of normal cells, can be used for detection of apoptosis.

The removal of dead cells, such as via apoptotic bodies, can be performed by an antigen presenting cell (APC). The APC can be a phagocytic cell or phagocyte. For example, the APC or phagocyte can be a macrophage. The macrophage can be identified by specific expression of one or more of the following markers, such as, but not limited to CD14, CD11b, F4/80 (mice)/EMR1 (human), Lysozyme M, MAC-1/MAC-3, and CD68. Identification can be by any means known in the art, such as by flow cytometry or immunohistochemical staining.

The apoptotic body can exhibit one or more molecules or markers that mark the apoptotic body for phagocytosis by cells possessing the appropriate receptors, such as an APC or macrophage. Without being bound by any particular theory, upon recognition, the phagocyte typically reorganizes its cytoskeleton for engulfment of the apoptotic body, thereby removing the dying cell, which is believed to occur in an orderly manner without eliciting an inflammatory response.

After digestion, a macrophage can present the antigen of the apoptotic body to the corresponding helper T cell. The presentation can be performed by integrating the antigen into the cell membrane of the macrophage and displaying the antigen attached to an MHC class II molecule, which indicates to other white blood cells that the macrophage is not a pathogen, despite having antigens on its surface. In some embodiments, an apoptotic body picked up by an antigen presenting cell, such as a host antigen presenting cell in the spleen, can induce tolerance. This presentation of the antigen to host T-cells in a non-immunogenic fashion can lead to direct induction of anergy.

The composition of the present invention may be chosen to maximize delivery to locations in where lymphocytes, such as immature lymphocytes, can be found. For example, the apoptotic body may be delivered to the spleen, thymus, bone marrow or lymph nodes. In one embodiment, the apoptotic body disclosed is targeted to the spleen, such as the marginal zone of the spleen. The apoptotic body can be carrying or associated with an antigen. In some instances, the antigen is delivered to antigen presenting cells (APCs), such as dendritic cells (DCs) or macrophages, where lymphocytes are undergoing maturation (e.g., spleen, bone marrow, thymus and lymph nodes). These are resident APCs and DCs, for example, in spleen, bone marrow, thymus and lymph nodes. Alternatively, the antigen-specific peptide may be delivered to peripheral APCs or DCs, where they first internalize the carriers and then migrate to sites of lymphocyte maturation (e.g., spleen, bone marrow, thymus and lymph nodes) to activate a tolerance response. Resident APCs at sites of lymphocyte maturation may be utilized as targets.

In another embodiment, the apoptotic body disclosed herein comprises one or more proteins or markers that allow it to be specifically bound or engulfed by a macrophage. In one embodiment, the macrophage is within the spleen. The macrophage may be a F4/80 macrophage. The macrophage may comprise one or more specific receptors, such as a scavenger receptor. The scavenger receptor can be CD68, LOX-1, SRB1, SRBII, or MARCO. In one particular embodiment, the scavenger receptor is SRBII or MARCO. The scavenger receptor, such as MARCO, may function through its ability to uptake particles, e.g., Ag-linked particles and assist in macrophage antigen presentation or antigen transfer to local dendritic cells. MARCO or other scavenger receptors may also inhibit inflammatory responses by preventing dendritic migration or by other unknown anti-inflammatory mechanisms.

The apoptotic body may have a specific size, such as less than about 1,000 μm, 500 μm, 100 μm, 50 μm, 25 μm, 20 μm, 15 μm, 10 μm, 5 μm, 1 μm, 500 nm, 400 nm, 300 nm, 200 nm, 100 nm, or 50 nm such as in diameter or across the widest point of the body. The apoptotic body can be between 5 nm and 10 μm in diameter, between 50 nm and 1 μm, between 100 nm and 1 μm, between 250 nm and 750 nm, between 300 nm and 700 nm, or between 400 nm and 600 nm. In another embodiment, apoptotic body can be about 500 nm in diameter. In some embodiments, the apoptotic body has a maximum diameter of about 500-800 nm. Alternatively, the apoptotic body may have a maximum diameter of about 100-700 nm, 200-600 nm, or 300-500 nm. In some embodiments, the overall mass of the apoptotic body is less than about 10,000 kDa, less than about 5,000 kDa, or less than about 1,000, 500, 400, 300, 200 or 100 kDa.

Also provided herein is an apoptotic body surrogate. The apoptotic body surrogate mimics an apoptotic body or debris from an apoptotic cell death such that they are recognized by an APC, such as a host APC or macrophage. In one aspect, an apoptotic body surrogate carrying an epitope of an antigen can be used to induce tolerance to antigen in a subject. The apoptotic body surrogate can be localized to the spleen and induce tolerance, such as an apoptotic body disclosed herein. For example, an apoptotic body surrogate comprises one or more of the characteristics of an apoptotic body, such as described above. The apoptotic body surrogate can have the same localization pattern of an apoptotic body, such as to the spleen, in particular the marginal zone of the spleen. In another embodiment, the apoptotic body surrogate is uptaken by a macrophage, such as disclosed herein. For example, the macrophage can comprise a SRBII or MARCO. The scavenger receptor, such as MARCO, may function through its ability to uptake particles, e.g., Ag-linked particles and assist in macrophage antigen presentation or antigen transfer to local dendritic cells. MARCO or other scavenger receptors may also inhibit inflammatory responses by preventing dendritic cell migration or by other unknown anti-inflammatory mechanisms.

The overall size and/or weight of the apoptotic body surrogate may be microscopic or nanoscopic in size, to enhance solubility and avoid possible complications caused by aggregation in vivo. The size of the apoptotic body surrogate can be similar or resemble that of an apoptotic body, such as described herein. For example, the apoptotic body surrogate can be less than about 1,000 μm, 500 μm, 100 μm, 50 μm, 25 μm, 20 μm, 15 μm, 10 μm, 5 μm, 1 μm, 500 nm, 400 nm, 300 nm, 200 nm, 100 nm, or 50 nm such as in diameter or across the widest point of the body. The apoptotic body surrogate can be between 5 nm and 10 μm in diameter, between 50 nm and 1 μm, between 100 nm and 1 μm, between 250 nm and 750 nm, between 300 nm and 700 nm, or between 400 nm and 600 nm. In another embodiment, apoptotic body surrogate can be about 500 nm in diameter. In some embodiments, the apoptotic body surrogate has a maximum diameter of about 500-800 nm. Alternatively, the apoptotic body surrogate may have a maximum diameter of about 100-700 nm, 200-600 nm, or 300-500 nm. In some embodiments, the overall mass of the apoptotic body surrogate is less than about 10,000 kDa, less than about 5,000 kDa, or less than about 1,000, 500, 400, 300, 200 or 100 kDa.

The apoptotic body surrogate can comprise a particle, bead, branched polymer, dendrimer, or liposome. The apoptotic body surrogate can comprise a quantum dot, dendrimer, liposome, micelle, nanoparticle or microparticle. The apoptotic body surrogate can be particulate. The apoptotic body surrogate can be generally spherical, ellipsoidal, rod-shaped, globular, or polyhedral in shape. The apoptotic body surrogate can be porous. Alternatively, the apoptotic body surrogate may be of an irregular or branched shape. The apoptotic body surrogate can be biodegradable. The apoptotic body surrogate can have a net neutral or negative charge, such as to reduce non-specific binding to cell surfaces which, in general, bear a net negative charge.

The surface of an apoptotic body surrogate can be composed of a material that minimizes non-specific or unwanted biological interactions. The surface may be coated with a material to prevent or decrease non-specific interactions. Steric stabilization by coating with hydrophilic layers such as poly(ethylene glycol) (PEG) and its copolymers such as PLURONICS (including copolymers of poly(ethylene glycol)-bl-poly(propylene glycol)-bl-poly(ethylene glycol)), may be used. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides. Many peptide sequences subject to specific enzymatic attach are known, e.g., as degraded by collagenases or metalloproteinases: sequences that are degraded merely by biological free radical mechanisms are not specifically degraded. Polymers with functional groups that are oxidation-sensitive can be chemically altered by mind oxidizing agents, with a test for the same being enhanced solubilization by exposure to 10% hydrogen peroxide for 20 h in vitro.

Another physical property is the apoptotic body surrogate surface's hydrophilicity. Apoptotic Body Surrogates (ABSs) can be made of a hydrophilic material having a solubility in water of at least 1 gram per liter when it is uncrosslinked. In one aspect, an ABS comprises a hydrophilic component, e.g., a layer of hydrophilic material. Examples of suitable hydrophilic materials are one or more of polyalkylene oxides, polyethylene oxides, polysaccharides, polyacrylic acids, and polyethers. The molecular weight of polymers in a layer can be adjusted to provide a useful degree of steric hindrance in vivo, e.g., from about 1,000 to about 100,000 or even more; artisans will immediately appreciate that all the ranges and values within the explicitly stated ranges are contemplated, e.g., between 10,000 and 50,000.

Other properties related to apoptotic body surrogate can include mechanical properties such as rigidity or rubberiness. In some embodiments, ABSs comprise a non-rubbery core. In another embodiment, the apoptotic body surrogate has a rubbery core, e.g., a poly(propylene sulfide) (PPS) core with an overlayer, e.g., a hydrophilic overlayer, as in PEG, as in the PPS-PEG system recently developed and characterized for systemic (but not targeted or immune) delivery. The rubbery core is in contrast to a substantially rigid core as in a polystyrene or metal nanoparticle system. The term rubbery refers to certain resilient materials besides natural or synthetic rubbers, with rubbery being a term familiar to those in the polymer arts. For example, cross-linked PPS can be used to form a hydrophobic rubbery core. PPS is a polymer that degrades under oxidative conditions to polysulfoxide and finally polysulfone, transitioning from a hydrophobic rubber to a hydrophilic, water-soluble polymer. Other sulfide polymers may be adapted for use, with the term sulfide polymer referring to a polymer with a sulfur in the backbone of the polymer. Other rubbery polymers that may be used are polyesters with glass transition temperature under hydrated conditions that is less than about 37° C. A hydrophobic core can be advantageously used with a hydrophilic overlayer since the core and overlayer will tend not to mingle, so that the overlayer tends to stericly expand away from the core. A core refers to a particle that has a layer on it. A layer refers to a material covering at least a portion of the core. A layer may be absorbed or covalently bound. A particle or core may be solid or hollow. Rubbery hydrophobic cores are advantageous over rigid hydrophobic cores, such as crystalline or glassy (as in the case of polystyrene) cores, in that higher loadings of hydrophobic drugs can be carried by the particles with the rubbery hydrophobic cores.

In another embodiment, the apoptotic body surrogate has a loading characteristic, such as a loading capability of at least about 50 μmole per gram of bead; of at least about 100 μmole per gram of bead; of at least about 150 μmole per gram of bead; of at least about 200 μmole per gram of bead; of at least about 250 μmole per gram of bead; of at least about 300 μmole per gram of bead; of at least about 350 μmole per gram of bead; of at least about 400 μmole per gram of bead; of at least about 450 μmole per gram of bead.

In some embodiments, a composition disclosed herein comprises a plurality of apoptotic body surrogates, with general uniformity in size distribution, pore size, density, swelling properties and/or tolerance to solvents and reagents typically used in oligomer synthesis.

The apoptotic body surrogate can be formed from a wide range of materials. The apoptotic body surrogate is preferably composed of a material suitable for biological use. For example, the apoptotic body surrogate may be composed of glass, silica, polyesters of hydroxy carboxylic acids, polyanhydrides of dicarboxylic acids, copolymers of hydroxy carboxylic acids and dicarboxylic acids, or any combination thereof. More generally, the apoptotic body surrogate may be composed of one or more polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, heteroaryl, or alkoxy dicarboxylic acids.

Additionally, the apoptotic body surrogate can comprise a quantum dot, such as quantum dot polystyrene particles (Joumaa et al., (2006) Langmuir 22:1810-6). The apoptotic body surrogate can comprise mixtures of ester and anhydride bonds (e.g., copolymers of glycolic and sebacic acid). For example, the apoptotic body surrogate can comprise materials including, but not limited to, polyglycolic acid polymers (PGA), polylactic acid polymers (PLA), polysebacic acid polymers (PSA), poly(lactic-co-glycolic) acid copolymers (PLGA), poly(lactic-co-sebacic) acid copolymers (PLSA), poly(glycolic-co-sebacic) acid copolymers (PGSA), polylactide co-glycolide (PLG), chitosan, or hyaluronic acid.

ABSs can be made of, in part or in whole, biocompatible or biodegradable polymers including polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include PLA, PGA, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON@ (DuPont, Wilmington, Del.), and nylons may be employed. The apoptotic body surrogate can also comprise polystyrene. For example, the apoptotic body surrogate can comprise a polystyrene bead, such as those commercially available (for example, FluoSpheres (Molecular Probes, Eugene, Oreg.)).

Polystyrene beads can be used to be an apoptotic body surrogate of the present invention. For example, a polystyrene bead of approximately 500 nm localizes to the marginal zone of the spleen and can induce tolerance to an antigen attached with the bead. In one embodiment, an apoptotic body surrogate composition comprises a crosslinked, functionalized polystyrene beads, with general uniformity in bead size distribution, pore size, density, swelling properties and/or tolerance to solvents and reagents typically used in oligomer synthesis. In another embodiment, the beads have superior loading characteristics, such as a loading capability of at least about 50 μmole per gram of bead; of at least about 100 μmole per gram of bead; of at least about 150 μmole per gram of bead; of at least about 200 μmole per gram of bead; of at least about 250 μmole per gram of bead; of at least about 300 μmole per gram of bead; of at least about 350 μmole per gram of bead; of at least 400 μmole per gram of bead; or at least about 450 μmole per gram of bead. In some embodiments, the bead has a loading capability of from about 100 μmole per gram of bead to about 350 μmole per gram of bead.

In some embodiments, the tolerance inducing compositions of the present invention comprises an apoptotic body or apoptotic body surrogate comprising a branched polymer, such as a dendrimer. Branched polymers have numerous chain-ends or termini which can be functionalized and, therefore, can be conjugated to a multiplicity of epitopes, either directly or indirectly through conjugating moieties. These polymers can comprise a high number of functional groups at their surface, for example which have been used to conjugate to biomolecules and other groups. Analogously, antigens could be conjugated to the dendrimer surface. Moreover, the functional groups on the dendrimer surface could be optimized for complement activation, for example by hydroxylation. Some dendrimer-DNA complexes have been demonstrated to activate complement. Dendrimers represent an interesting nanoparticulate chemistry that could be adapted for lymphatic targeting using the techniques described herein, for antigen conjugation, and for complement activation, e.g., as in U.S. Pat. Pub. Nos. 2004/0086479, 2006/0204443, and in U.S. Pat. Nos. 6,455,071 and 6,998,115, which are hereby incorporated by reference herein to the extent they do not contradict what is explicitly disclosed.

Dendrimers, also known as arborols, cascade molecules, dendritic polymers, or fractal polymers, are highly branched macromolecules in which the branches emanate from a central core. Dendrimers can have a shape that is highly dependent on the solubility of its component polymers in a given environment, and can change dramatically according to the solvent or solutes around it, e.g., changes in temperature, pH, ion content, or after uptake by a DC. Dendrimers can be made from various materials, including, but not limited to, polyamidoamine, polyamidoalcohol, polyalkyleneimine such as polypropyleneimine or polyethyleneimine, polyalkylene such as polystyrene or polyethylene, polyether, polythioether, polyphosphonium, polysiloxane, polyamide, polyaryl polymer, or combinations thereof. Dendrimers have also been prepared from amino acids (e.g., polylysine). Preferably, dendrimers are employed which terminate in carboxyl or other negatively charged reactive groups in order to facilitate conjugation.

Dendrimers are known in the art and are chemically defined globular molecules, generally prepared by stepwise or reiterative reaction of multifunctional monomers to obtain a branched structure (see, e.g., Tomalia et al., (1990) Angew. Chem Int. Ed. Engl. 29:138-75). A variety of dendrimers are known, e.g., amine-terminated polyamidoamine, polyethyleneimine and polypropyleneimine dendrimers. Exemplary dendrimers useful in the present invention include “dense star” polymers or “starburst” polymers such as those described in U.S. Pat. Nos. 4,587,329; 5,338,532; and 6,177,414, including poly(amidoamine) dendrimers (“PAMAM”). Still other multimeric spacer molecules suitable for use within the present invention include chemically-defined, non-polymeric valency platform molecules such as those disclosed in U.S. Pat. No. 5,552,391; and PCT application publications WO 00/75105, WO 96/40197, WO 97/46251, WO 95/07073, and WO 00/34231. Many other suitable multivalent spacers can be used and will be known to those of skill in the art. For example, dendrimers and their use are described in U.S. Pat. Appl. No. 20070238678, which is hereby incorporated by reference in its entirety.

Such dendrimers include but are not limited to polyamidoamine (PAMAM) dendrimers, poly(propyleneimine) (PPI) dendrimers, poly(triazine) dendrimers, poly(ether-hydroxylamine) (PEHAM) dendrimers, which may have their Z groups modified or selected to force the chelating agents exclusively into the dendritic polymer interior or in combination with encapsulation, allow association with the surface of the dendritic polymer. Examples of some such Z surfaces are those which do not interact with the ligand; such Z groups are hydroxyl, ester, acid, ether, carboxylic salts, alkyls, glycols, such as for example hydroxyl groups especially those from amidoethanol, amidoethylethanolamine, tris(hydroxymethyl)amine, carbomethoxypyrrolidinone, amido, thiourea, urea, carboxylate, succinamic acid and polyethylene glycol or primary, secondary or tertiary amine groups with or without hydroxyl alkyl modifications. Other suitable surface groups may include any such functionality that would allow associative attachment (associate with) the dendritic polymer surface and include but are not limited to receptor mediated targeting groups (e.g., folic acid, antibodies, antibody fragments, single chain antibodies, proteins, peptides, oligomers, oligopeptides, or genetic materials) or other functionality that would facilitate biocompatibility, biodistribution, solubility or modulate toxicity. In a preferred embodiment, the dendrimers contain amino and/or carboxy binding sites on the surface.

In one embodiment, an apoptotic body surrogate comprises a commercially available dendrimer, such as, but not limited to a polyamidoamine dendrimer such as a Starburst™ dendrimer (Dendritech, Midland, Mich.). The Starburst™ dendrimers terminate in either amine groups or carboxymethyl groups which may be used, with or without further modification, and with or without interposing conjugating moieties, to conjugate antigen peptides and proteins to the surface of these carriers.

In one method of dendrimer production, dendrimers are synthesized outward from a core molecule by sequential addition of layers of monomers. The first round of dendrimer synthesis adds a single layer or “generation” of monomers to the core, with each monomer having at least one free, reactive terminus. Each subsequent round of polymerization results in the expansion of the dendrimer by one layer and increases the number of free, reactive termini. This process can be repeated numerous times to produce dendrimers of desired diameter and mass. As the density of the branches increases, the outermost branches arrange themselves in the form of a sphere surrounding a lower density core. See, for example, U.S. Pat. No. 5,338,532, which is hereby incorporated by reference in its entirety. In addition, by varying the shape of the cored molecules, dendrimers may be produced in rod-shaped, disk-like, and comb-like forms. The resulting dendrimers may possess an arbitrarily large number of free, reactive termini, to which a multiplicity of antigen peptides and proteins may be conjugated, either directly or indirectly. In a preferred embodiment, the dendrimers are spherical or ovoid in shape.

Dendrimers may vary in weight, size, shape and number of terminal reactive groups. For example, dendrimers may range in weight from 100 to 10000 kDa, or 200 to 5000 kDa, or 250 to 2500 kDa. Dendrimers may also range in size from 20 to 1000 nm, 30 to 500 nm, or 50 to 250 nm in the longest dimension.

The use of dendrimers, e.g., PAMAM or PPI dendrimers, enables the creation of cationic spherical particles with a specific number of amino binding sites on the surface. The size of these particles can be selected to optimize loading and minimize steric hindrance between surface linked antigens or epitopes. For example, PAMAM dendrimers of 6-7 generations have been used resulting in particles of 50-125 kDa molecular weights, 60-90 angstrom diameter (roughly similar in size as hemoglobin, IgG or histones), and 100-1500 active surface groups.

Dendrimers of the present invention may be composed of a somewhat heterogeneous mixture of molecules produced, i.e., comprising different numbers (within or predominantly within a determinable range) of nucleic acid moieties joined to each dendrimer molecule. In a preferred embodiment, the dendrimers are of a similar size and shape, i.e., composed of numbers of nucleic acid moieties that vary within 20%, 15%, 10%, 5%, 2% or 1% of each other.

Non-dendrimer branched polymers may also be employed in the invention, and may be produced from the same general classes of materials as dendrimers. The synthesis of such branched polymers is also well-known in the art. Branched polymers may include at least 5 termini, at least 10 termini, or at least 100 termini. Branched polymers may include between 5 and 500 termini, preferably between 100 and 400 termini and more preferably between 50 and 250 termini. In some embodiments, the tolerance inducing compositions of the present invention provides for the production of conjugates wherein a tolerance inducing complex is conjugated to a branched or linear polymer.

In some embodiments, the apoptotic body surrogate comprises a liposome or micelle. Liposomes, also called lipid vesicles, are aqueous compartments enclosed by lipid membranes, and are typically formed by suspending a suitable lipid in an aqueous medium, and shaking, extruding, or sonicating the mixture to yield a dispersion of vesicles. Various forms of liposomes, including unilamellar vesicles and multilamellar vesicles, may be used in the present invention.

Micellar systems may also display the same useful characteristics as described above, including micelles formed from AB and ABA block copolymers of poly(ethylene glycol) and PPS. When such copolymers are formed with a molecular fraction of poly(ethylene glycol) that is relatively high, e.g., in excess of approx. 40%, then spherical micelles can be expected to form under certain conditions. These micelles can be small, e.g., meeting the size mentioned above, and may optionally be grafted with an overlayer of PEG, or otherwise incorporated PEG or other polymers to achieve similar properties. Moreover, they can be conjugated with antigen, as taught herein, danger signals or both at the micelle surface. The block copolymer can terminate in a hydroxyl group, for complement activation, and can be beneficial for having a hydrophilic block terminate in a hydroxyl group, so that this hydroxyl group is more readily available on the micellar surface for complement binding. Such hydroxylated surfaces can be tailored to effectively activate complement. A particularly useful hydrophilic block is PEG, terminated in a hydroxyl group. In additional to micelle-forming polymer architectures, block sizes and block size ratios can be selected to form vesicular structures. There also exist a number of other possible chemical compositions of micellar formulations that may be used.

Liposomes may be prepared from a variety of lipid materials including, but not limited to, lipids of phosphatidyl choline, phosphatidyl serine, phosphatidyl inositol, phosphatidyl glycerol, phosphatidyl ethanolamine, phosphatidic acid, dicetyl phosphate, monosialoganglioside, polyethylene glycol, stearyl armine, ovolecithin and cholesterol, as well as mixtures of these in varying stoichiometries. Liposomes, as used herein, may also be formed from non-lipid amphipathic molecules, such as block copolymers of poly(oxyethylene-b-isoprene-b-oxye-thylene) and the like. In preferred embodiments, the liposomes are prepared from lipids that will form negatively charged liposomes, such as those produced from phosphatidyl serine, dicetyl phosphate, and dimyristoyl phosphatidic acid.

The surfaces of liposomes may also be modified to reduce immunogenicity or to provide convenient reactive groups for conjugation. For example, sialic acid or other carbohydrates, or polyethylene glycol or other alkyl or alkenyl polymers, may be attached to the surface of a liposome to reduce immunogenicity. Alternatively, liposomes may be produced bearing a conjugating moiety such as biotin by inclusion of a small molar percentage of, for example, biotin-X-dipalmitoylphosphatidyle-thanolamine (Molecular Probes, Eugene, Oreg.) in the liposome.

The apoptotic body surrogate can also incorporate one or more functional groups for further reaction. Functional groups for further reaction include electrophiles or nucleophiles; these are convenient for reacting with other molecules, such as further one or more antigens or other molecules as described herein. Examples of nucleophiles are primary amines, thiols, and hydroxyls. Examples of electrophiles are succinimidyl esters, aldehydes, isocyanates, and maleimides.

### Linkages

The apoptotic body or surrogate thereof can be linked, attached or conjugated, either directly or indirectly, to one or more components. For example, in one embodiment, an antigen or a plurality of the same or different antigens is attached to a single or plurality of apoptotic bodies or surrogates thereof. In another embodiment, an epitope or a plurality of epitopes, from the same or different antigen, is attached to a single or plurality of apoptotic bodies or surrogates thereof. In yet another embodiment, the one or more epitopes can be immunodominant epitopes. The apoptotic body or surrogate thereof can be further attached to one or more additional molecules, such as, but not limited to, an anergy promoting agent, an apoptosis including molecule, a molecule recognized by a macrophage receptor (such as, but not limited to, a scavenger receptor).

The apoptotic body or surrogate thereof can have one or a plurality of attachment, linkage, or binding sites. The linkage can be covalent or non-covalent. The apoptotic body or surrogate thereof may have a surface to which conjugating moieties may be absorbed without chemical bond formation.

A great variety of means, well known in the art, may be used to link, attach or conjugate a molecule, such as an epitope of an antigen, to an apoptotic body or surrogate thereof. These methods include any standard chemistry which does not destroy or severely hinder the biological activity of the epitope or apoptotic body or surrogate thereof. The methods can permit a sufficient number of molecules, such as one or more immunodominant epitopes, to be attached or conjugated to one or more apoptotic bodies or surrogates thereof. In one embodiment, the molecule to be conjugated is in an orientation which allows for interaction of the epitope with a cognate T cell receptor.

In one embodiment, the C-terminal region of the antigen is attached to the carrier. The chemistry is dependent upon the nature of the carrier material, the presence or absence of C-terminal fusions to the antigen, and/or the presence or absence of conjugating moieties. In another embodiment, the N-terminal region of the antigen is attached to the carrier. The chemistries are dependent upon the nature of the carrier material, the presence or absence of N-terminal fusions to the antigen, and/or the presence or absence of conjugating moieties.

Functional groups can be located on the particle as needed for availability. One location can be as side groups or termini on the core polymer or polymers that are layers on a core or polymers otherwise tethered to the particle. For instance, examples are included herein that describe PEG stabilizing the apoptotic body or surrogate thereof that can be readily functionalized for specific cell targeting or protein and peptide drug delivery.

In one embodiment, a conjugate used for attaching one or more epitopes or antigens comprises an ethylene or carbodiimide conjugate. For example, conjugates such as ethylene carbodiimide (ECDI), hexamethylene diisocyanate, propyleneglycol di-glycidylether which contain 2 epoxy residues, and epichlorohydrin may be used for fixation of an antigen to the surface of an apoptotic body or surrogate thereof. Without being bound by theory, ECDI chemically couples an antigen to the cell surface via catalysis of peptide bond formation between free amino and free carboxyl groups; while also mimicking an apoptotic cell or body, thereby inducing recognition by an APC, such as an APC in the spleen or splenic marginal zone. The APC can present an epitope of the antigen to a host T-cell in a non-immunogenic fashion that leads to induction of anergy in autoreactive cells. In addition, ECDI may serve as a potent stimulus for the induction of specific regulatory T cells.

In one series of embodiments, the epitope is bound to an apoptotic body or surrogate thereof via a covalent chemical bond. For example, a reactive group or moiety near the C-terminus of the antigen comprising the epitope (e.g., the C-terminal carboxyl group, or a hydroxyl, thiol, or amine group from an amino acid side chain) may be conjugated directly to a reactive group or moiety on the surface of the apoptotic body or surrogate thereof (e.g., a hydroxyl or carboxyl group of a PLA or PGA polymer, a terminal amine or carboxyl group of a dendrimer, or a hydroxyl, carboxyl or phosphate group of a phospholipid) by direct chemical reaction. Alternatively, there may be a conjugating moiety which covalently conjugates to both the antigen and the apoptotic body or surrogate thereof, thereby linking them together.

Reactive carboxyl groups on the surface of an apoptotic body or surrogate thereof may be joined to one or more free amines (e.g., from Lys residues) on the antigen or epitope, by reacting them with, for example, 1-ethyl-3-[3,9-dimethyl aminopropyl] carbodiimide hydrochloride (EDC) or N-hydroxysuccinimide ester (NHS). Similarly, the same chemistry may be used to conjugate free amines on the surface of an apoptotic body or surrogate thereof with one or more free carboxyls (e.g., from the C-terminus, or from Asp or Glu residues) on the antigen or epitope. Alternatively, free amine groups on the surface of an apoptotic body or surrogate thereof may be covalently bound to an epitope or antigen using sulfo-SIAB chemistry, such as described by Arano et al., (1991) Bioconjug. Chem. 2:71-6.

In another embodiment, a non-covalent bond between a ligand bound to an antigen and an anti-ligand attached to an apoptotic body or surrogate thereof may conjugate the epitope of the antigen to the apoptotic body or surrogate thereof. For example, a biotin ligase recognition sequence tag may be joined to the C-terminus of an antigen, and this tag may be biotinylated by biotin ligase. The biotin may then serve as a ligand to non-covalently conjugate the antigen to avidin or streptavidin which is absorbed or otherwise bound to the surface of the carrier as an anti-ligand. Alternatively, if the antigen is fused to an immunoglobulin domain bearing an Fc region, the Fc domain may act as a ligand and protein A, either covalently or non-covalently bound to the surface of the carrier, may serve as the anti-ligand to non-covalently conjugate the antigen to the apoptotic body or surrogate thereof. Other means are well known in the art which may be employed to non-covalently conjugate antigen peptides and proteins to carriers, including metal ion chelation techniques (e.g., using a poly-His tag at the C-terminus of the antigen peptide or protein or antigen peptide or protein fusion proteins, and a Ni⁺-coated carrier), and these methods may be substituted for those described here.

Conjugation of a nucleic acid moiety to a platform molecule can be effected in any number of ways, typically involving one or more crosslinking agents and functional groups on the nucleic acid moiety and platform molecule. Linking groups are added to platforms using standard synthetic chemistry techniques. Linking groups can be added to nucleic acid moieties using standard synthetic techniques.

### Epitopes and Antigens

The present invention provides compositions and methods for inducing antigen-specific tolerance in a subject comprising an apoptotic body, or apoptotic body surrogate, and an epitope of an antigen. The antigen is a tolerance inducing antigen that contributes to the specificity of the tolerogenic response that is induced. The one or more antigens can act as an allergen that would otherwise induce T-cell receptor-mediated stimulation in a subject (i.e., if the subject had not been administered a composition comprising an apoptotic body, or apoptotic body surrogate, and an epitope of an antigen). In another embodiment, the antigen is not the same as the target antigen, wherein the target antigen is associated with a condition or suspected to cause a condition in a subject, wherein the target antigen can act as an allergen that would otherwise induce T-cell receptor-mediated stimulation in a subject (i.e., if the subject had not been administered a composition comprising an apoptotic body, or apoptotic body surrogate, and an epitope of an antigen).

In one embodiment, a composition can comprise a plurality of different antigens associated with the same condition. For example, the composition may comprise different antigens associated with multiple sclerosis. In another embodiment, a composition can comprise a plurality of different antigens associated with the same general condition. For example, the composition may comprise different antigens, each antigen being from a different plant, associated with a pollen allergen. In another embodiment, the composition may comprise different food antigens. In another embodiment, the composition comprises a plurality of antigens, wherein a subset of the plurality is associated with one condition and another subset is associated with a second condition.

In another embodiment, the composition comprises a plurality of different epitopes or fragments from the same antigen associated or suspected to cause a condition. In yet another embodiment, the composition comprises a plurality of different epitopes from a plurality of different antigens. The different epitopes can be immunodominant epitopes. An immunodominant epitope is a subunit of an antigen or antigen determinant that is most easily recognized by the immune system, such that the immunodominant epitope is responsible for the major immune response in a host to the antigenic determinant. The immunodominant epitope is also thought to most influence the specificity of an antibody to the epitope. Immunodominant epitopes have been identified for numerous antigens, such as described in Ota et al., Nature 346, 183-187 (1990) and Slavin et al., Autoimmunity 28, 109-120 (1998). Methods of identifying an immunodominant epitope is also known in the art, such as described in Kuwana et al., Arthritis Rheum. 46, 2742-7 (2002) and Huard et al., Int. Immunol. 9, 1701-7 (1997). The method can comprise generating overlapping regions of an antigen and determining the specificity of an antibody to each region. For example, overlapping peptides of an antigen can be generated and epitope specificity to an antibody for the antigen is determined using ELISA. The peptides can comprise at least 3, 4, 5, 6, 7, 8, 9, 10, 11, 12, 13, 14, 15, 16, 17, 18, 19, 20, 21, 22, 23, 24, or 25 amino acids, wherein the overlap between the peptides can be at least 1, 2, 3, 4, 5, 6, 7, 8, 9, 10, 11, 12, 13, 14, 15, 16, 17, 18, 19, 20, 21, 22, 23, 24, or 25 amino acids. Sera from a subject can be used to test the specificity, such as sera from a subject with a condition.

In one embodiment, an immunodominant epitope is identified and used in a composition disclosed herein. In another embodiment, the immunodominant epitope is known in the art. In yet another embodiment, the immunodominant epitope is of a myelin protein, such as MBP 13-32: KYLATASTMDHARHGFLPRH (SEQ ID NO: 1), MBP 83-99: ENPWHFFKNIVTPRTP (SEQ ID NO: 2), MBP 111-129: LSRFSWGAEGQRPGFGYGG (SEQ ID NO: 3), MBP 146-170: AQGTLSKIFKLGGRDSRSGSPMARR (SEQ ID NO: 4), PLP 139-154: IICLGKWLGHPDKFVGI (SEQ ID NO: 5), MOG 1-20: GQFRVIGPRHPIRALVGDEV (SEQ ID NO: 6), MOG 35-55: MEVGWYRPPFSRWHLYRNGK (SEQ ID NO: 7), or MBP 82-98: DENPWHFFKNIVTPRT (SEQ ID NO: 8).

In one embodiment, a composition comprises one or more of the immunodominant epitopes, wherein each is attached to an apoptotic body or surrogate thereof. In another embodiment, a composition comprises a plurality of immunodominant epitopes, wherein the plurality is attached to a single apoptotic body or surrogate thereof. In another embodiment, a composition comprises at least 2, 3, 4, 5, 6, 7, 8, 9, 10, 11, 12, 13, 14, 15, 16, 17, 18, 19, or 20 immunodominant epitopes, wherein each is attached to an apoptotic body or surrogate thereof. In yet another embodiment, a composition comprises at least 2, 3, 4, 5, 6, 7, 8, 9, 10, 11, 12, 13, 14, 15, 16, 17, 18, 19, or 20 immunodominant epitopes, wherein a plurality, such as at least 2, 3, 4, 5, 6, 7, 8, 9, 10, 11, 12, 13, 14, 15, 16, 17, 18, 19, or 20 immunodominant epitopes is attached to a single apoptotic body or surrogate thereof. The immunodominant epitopes can be different or the same.

The composition can comprise a plurality of immunodominant epitopes, wherein at least one of the plurality is associated with first antigen and another one of the plurality is associated with a second antigen. The first and second antigen can be associated with the same condition, such as multiple sclerosis. In another embodiment, the first and second antigen can be associated with the same general condition, such as pollen allergy (for example, the first antigen can be of a first seed plant, and the second is of a second seed plant). In another embodiment, the first and second antigen can be different food allergens. In yet another embodiment, the first and second antigen can each be associated with different conditions.

The epitope, such as immunodominant epitope, can be from an antigen comprising a molecule isolated or derived from a biological source, such as a polypeptide, polynucleotide, carbohydrate, glycolipid, or any combination thereof. Alternatively, the antigen can comprise a molecule that is chemically synthesized, such as a small molecule, or a synthetic polypeptide, polynucleotide, carbohydrate, glycolipid, or any combination thereof. In certain embodiments of this invention, the inducing antigen is a single isolated or recombinantly produced molecule. For treating conditions where the target antigen that is associated or suspected to cause a condition is disseminated to various locations in the host, the inducing antigen may be identical to or immunologically related to the target antigen. Examples of such antigens are most polynucleotide antigens and some carbohydrate antigens (such as blood group antigens).

In another embodiment, where the target antigen is preferentially expressed on a particular organ, cell, or tissue type, an inducing antigen which is identical with or immunologically related to the target antigen can be used in a composition disclosed herein. However, an antigen which is a bystander for the target antigen can also be used. This is an antigen which may not me immunologically related to the target antigen, but is preferentially expressed in a tissue where the target antigen is expressed. A working theory as to the effectiveness of bystander suppression is that suppression is an active cell-mediated process that down-regulates the effector arm of the immune response at the target cells. The suppressor cells are specifically stimulated by the inducer antigen at the mucosal surface, and home to a tissue site where the bystander antigen is preferentially expressed. Through an interactive or cytokine-mediated mechanism, the localized suppressor cells then down-regulate effector cells (or inducers of effector cells) in the neighborhood, regardless of what they are reactive against. If the effector cells are specific for a target different from the inducing antigen, then the result is a bystander effect. In one embodiment, one of ordinary skill need not identify or isolate a particular target antigen against which tolerance is desired in order to practice the present invention, in that a molecule preferentially expressed at the target site can be used as an inducing antigen.

In certain embodiments of this invention, the inducing antigen is not in the same form as expressed in the individual being treated, but is a fragment or derivative thereof. Inducing antigens include, but are not limited to, peptides based on a molecule of the appropriate specificity but adapted by fragmentation, residue substitution, labeling, conjugation, and/or fusion with peptides having other functional properties. The adaption may be performed for any desirable purposes, including but not limited to the elimination of any undesirable property, such as toxicity or immunogenicity; or to enhance any desirable property, such as mucosal binding, mucosal penetration, or stimulation of the tolerogenic arm of the immune response. Terms such as insulin peptide, collagen peptide, and myelin basic protein peptide, as used herein, refer not only to the intact subunit, but also to allotypic and synthetic variants, fragments, fusion peptides, conjugates, and other derivatives that contain a region that is homologous (preferably 70% identical, more preferably 80% identical and even more preferably 90% identical at the amino acid level) to at least 10 and preferably 20 consecutive amino acids of the respective molecule for which it is an analog, wherein the homologous region of the derivative shares with the respective parent molecule an ability to induce tolerance to the target antigen.

The antigen may comprise a component of an allergen. In one embodiment, administration of a composition comprising an apoptotic body or surrogate thereof and an epitope of an allergen, such as an immunodominant epitope, induces tolerance to the allergen in a subject. The allergen can be, but not limited to, an animal product, drug or therapeutic, food, insect or insect product, fungus, plant, or non-biological product. For example, the animal product can be Fel d 1, a component of fur or dander, or dust mite. In another embodiment, the insect can be a cockroach, ant, bee, wasp, or mosquito, product therefrom. Non-biological products can include, but not be limited to, latex or a metal.

In one embodiment, the allergen is a food. The administration of a composition comprising an apoptotic body or surrogate thereof and an epitope of a food, such as an immunodominant epitope, can reduce a hypersensitivity response of a food allergy in the subject. A composition comprising an apoptotic body or surrogate thereof and an epitope of a food allergen (such as an immunodominant epitope of the food) can be administered to a subject, thereby inducing tolerance of the food in the subject, whereby the subject's contact with the food would otherwise induce T-cell receptor-mediated stimulation in the subject.

The antigen may comprise a component of a food that causes a hypersensitive response in a subject. For example, the antigen may comprise a component of, but not limited to, soy, wheat, fish, shellfish, fruit, vegetable, spice, synthetic or natural color, chicken, garlic, oat, and chemical additive (such as MSG or a sulphite). For example, a composition disclosed herein can comprise an apoptotic body or surrogate thereof with an epitope from such an antigen. The antigen may comprise an epitope, such as an immunodominant epitope, of a component, such as a protein, from a fruit or nut. The antigen may comprise an antigen from a peanut, or a tree nut, such as a pecan, pistachio, pine nut, or walnut. In another embodiment, the antigen may comprise a component of a seed, such as comprising an epitope, such as an immunodominant epitope, of a component, such as a protein or oil, from a sesame seed or poppy seed. The antigen may comprise an antigen from an egg. The antigen may comprise a component of an egg yolk or egg white, such as comprising an epitope, such as an immunodominant epitope, of a component of an egg. The component may be a protein, such as albumen. The antigen may comprise a component of honey, for example comprising an epitope, such as an immunodominant epitope, of a component of honey. In another embodiment, the food is celery or celeriac, corn or maize, pumpkin, a legume (such as a bean, pea, or soybean), a fruit (such as banana, avocado, kiwi, or chestnut), a grain, meat product (such as beef), or a diary product. For example, the antigen may comprise a component of lactose, thereby administrating a composition comprising an apoptotic body or surrogate thereof with an epitope or antigen of lactose can induce tolerance of diary products in a subject with lactose intolerance. In another embodiment, the antigen comprises a component of gluten, thereby inducing tolerance of products with gluten in a subject with gluten intolerance or allergies.

In yet another embodiment, an antigen can be a component of a plant, such as poison ivy, eastern poison oak, western poison oak or poison sumac. In yet another embodiment, the antigen comprises a component of pollen, such as a grass, weed, or tree. For example, the grass may be a ryegrass or timothy-grass. The weed can be a ragweed, plantago, nettle, Artemisia vulgaris Chenopodium album, or sorrel. The tree can be a birch, alder, hazel, hornbeam, aesculus, willow, poplar, platanus, tilia, olea, or juniper (such as an Ashe juniper).

In yet another embodiment, an antigen can be a component of an animal, such as venom from a snake or a bee, such as honey bee, for example comprising an epitope, such as an immunodominant epitope, of a component of bee sting or snake sting.

In another embodiment, a therapeutic is an allergen. The therapeutic can act as an allergen that would otherwise induce T-cell receptor-mediated stimulation in a subject that had not been administered a composition comprising an apoptotic body or surrogate thereof, with an epitope from the therapeutic, such as an immunodominant epitope. The therapeutic can be a drug, such as a small molecule, antibody, nucleic acid, or peptide. For example, the therapeutic can comprise an antibody or fragment thereof. In another embodiment, the therapeutic comprises tetracycline, Dilatin, carbamzepine, cephalosporin, penicillin, sulfonamide, steroid, non-steroidal anti-inflammatory, or salicylate. In another embodiment, the allergen is a reagent used in surgery or medical procedures, such as I.V contrast dye or anesthetic.

In yet another embodiment, the antigen is a component of a tissue to be transplanted. The antigen can comprise an allogeneic cell extract or endothelial cell antigen. For example, an apoptotic body or surrogate thereof, and an epitope of a tissue to be transplanted, allogeneic cell extract or endothelial cell antigen, such as an immunodominant epitope, can be administered to a subject prior, concurrent, or subsequent to receiving the tissue, such that the composition induces tolerance of the tissue in the subject thereby reducing the risk of transplant rejection in the subject or increasing transplant tolerance. The tissue may act as an allergen that would otherwise induce T-cell receptor-mediated stimulation in the subject, such as if the subject were not administered the composition comprising an apoptotic body or surrogate thereof, and an epitope of a tissue to be transplanted. The tissue can be any transplanted tissue or organ, including, but not limited to, heart, heart valve, liver, lung, kidney, intestine, skin, eye, cornea, pancreas, ligament, tendon, and bone, composite tissue grafts (e.g., hand transplant, face transplant) and multiple organ transplants (e.g., heart-lung transplants, kidney-pancreas transplants).

The composition can further comprise an immunosuppressive agent, such as one known in the art. For example, it can be selected from the group consisting of, but not limited to, cyclosporins or metabolites or synthetic analogues thereof (such as Cyclosporin A), tacrolimus, rapamycin, corticosteroids, cyclophosphamide, chlorambucil, azathioprine, myclophenolate mofetil.

In one embodiment, a composition disclosed herein comprises one or more of antigens, wherein each is attached to an apoptotic body or surrogate thereof. In another embodiment, a composition comprises a plurality of antigens, wherein the plurality is attached to a single apoptotic body or surrogate thereof. In another embodiment, a composition comprises at least 2, 3, 4, 5, 6, 7, 8, 9, 10, 11, 12, 13, 14, 15, 16, 17, 18, 19, or 20 antigens, wherein each is attached to an apoptotic body or surrogate thereof. In yet another embodiment, a composition comprises at least 2, 3, 4, 5, 6, 7, 8, 9, 10, 11, 12, 13, 14, 15, 16, 17, 18, 19, or 20 antigens, wherein a plurality, such as at least 2, 3, 4, 5, 6, 7, 8, 9, 10, 11, 12, 13, 14, 15, 16, 17, 18, 19, or 20 antigens is attached to a single apoptotic body or surrogate thereof. The antigens can be different or the same.

For example, both insulin and glucagon can be mixed with a mucosal binding component in the treatment of diabetes. It may also be desirable to provide a cocktail of antigens to cover several possible alternative targets. For example, a cocktail of histocompatibility antigen fragments could be used to tolerize a subject in anticipation of future transplantation with an allograft of unknown phenotype. Allovariant regions of human leukocyte antigens are known in the art: e.g., Immunogenetics 29:231, 1989. In another example, a mixture of allergens may serve as inducing antigen for the treatment of atopy.

Notably, even in diseases where the pathogenic autoantigen is unknown, bystander suppression may be induced using antigens present in the anatomical vicinity. For example, autoantibodies to collagen are observed in rheumatoid arthritis and, accordingly, a collagen-encoding gene may be utilized as the antigen-expressing gene module in order to treat rheumatoid arthritis (see e.g. Choy (2000) Curr Opin Investig Drugs 1:58-62). Furthermore, tolerance to beta cell autoantigens may be utilized to prevent development of type 1 diabetes (see e.g. Bach and Chatenoud (2001) Ann Rev Immunol 19:131-161).

As another example, auto-antibodies directed against myelin oligodendrocyte glycoprotein (MOG) are observed in autoimmune encephalomyelitis and in many other CNS diseases as well as multiple sclerosis (see e.g. Iglesias et al. (2001) Glia 36:22-34). Accordingly, use of MOG antigen expressing construct in the invention allows for treatment of multiple sclerosis as well as related autoimmune disorders of the central nervous system.

As yet another example, auto-antibodies directed against aquaporin 4 are observed in neuromyelitis optica (see e.g. Paul et al. (2007) PLos Med 4(4): e133. doi:10.1371/journal.pmed.0040133). Accordingly, use of aquaporin 4 antigen expressing constructs in the invention allows for treatment of neuromyelitis optica.

In one embodiment, the antigenic peptide or protein is an autoantigen, an alloantigen or a transplantation antigen. In yet another particular embodiment, the autoantigen is selected from the group consisting of myelin basic protein, collagen or fragments thereof, DNA, nuclear and nucleolar proteins, mitochondrial proteins and pancreatic β-cell proteins.

Still other examples of candidate autoantigens for use in treating autoimmune disease include: aquaporin 4 (see above) antigens to treat neuromyelitis optica; pancreatic beta-cell antigens, insulin and GAD to treat insulin-dependent diabetes mellitus; collagen type 11, human cartilage gp 39 (HCgp39) and gpl30-RAPS for use in treating rheumatoid arthritis; myelin basic protein (MBP), proteolipid protein (PLP) and myelin oligodendrocyte glycoprotein (MOG, see above) to treat multiple sclerosis; fibrillarin, and small nucleolar protein (snoRNP) to treat scleroderma; thyroid stimulating factor receptor (TSH-R) for use in treating Graves' disease; nuclear antigens; histones, glycoprotein gp70 and ribosomal proteins for use in treating systemic lupus erythematosus; pyruvate dehydrogenase dehydrolipoamide acetyltransferase (PCD-E2) for use in treating primary billiary cirrhosis; hair follicle antigens for use in treating alopecia areata; and human tropomyosin isoform 5 (hTM5) for use in treating ulcerative colitis.

Antigens can be prepared by a number of techniques known in the art, depending on that nature of the molecule. Polynucleotide, polypeptide, and carbohydrate antigens can be isolated from cells of the species to be treated in which they are enriched. Short peptides are conveniently prepared by amino acid synthesis. Longer proteins of known sequence can be prepared by synthesizing an encoding sequence or PCR-amplifying an encoding sequence from a natural source or vector, and then expressing the encoding sequence in a suitable bacterial or eukaryotic host cell.

In certain embodiments of this invention, the composition comprises a complex mixture of antigens obtained from a cell or tissue, one or more of which plays the role of inducing antigen. The antigens may be in the form of whole cells, either intact or treated with a fixative such as formaldehyde, glutaraldehyde, or alcohol. The antigens may be in the form of a cell lysate, created by detergent solubilization or mechanical rupture of cells or tissue, followed by clarification. The antigens may also be obtained by subcellular fractionation, particularly an enrichment of plasma membrane by techniques such as differential centrifugation, optionally followed by detergent solubilization and dialysis. Other separation techniques are also suitable, such as affinity or ion exchange chromatography of solubilized membrane proteins.

### Additional Agents

The present invention provides compositions and methods for inducing antigen-specific tolerance in a subject comprising an apoptotic body, or apoptotic body surrogate, an epitope of an antigen, and an additional agent, such as an anergy promoting agent or apoptotic signaling molecule. The components of the composition can be administered in one composition or administered as separate compositions to a subject. For example, a composition comprising an apoptotic body, or apoptotic body surrogate, and an epitope of an antigen and a composition comprising an anergy promoting agent is administered to a subject. In another embodiment, a composition comprising an apoptotic body, or apoptotic body surrogate, an epitope of an antigen and an anergy promoting agent and/or apoptotic signaling molecule is administered to a subject.

In some embodiments, the composition of the present invention comprises an apoptotic signaling molecule. The apoptotic signaling molecule can enhance the recognition by an APC of the apoptotic body, allowing presentation of the associated epitope, such as immunodominant epitope, in a tolerance-inducing manner. Without being bound by theory, this is presumed to prevent the upregulation of molecules involved in immune cell stimulation, such as MHC class I/II, and costimulatory molecules. These apoptosis signaling molecules may also serve as phagocytic markers. For example, apoptosis signaling molecules suitable for the present invention have been described in US Pat App No. 20050113297, which is hereby incorporated by reference in its entirety Molecules suitable for the present invention include molecules that target phagocytes, which include macrophages, dendritic cells, monocytes and neutrophils.

Molecules suitable as apoptotic signaling molecules can act to enhance tolerance of the associated epitope. Additionally, an apoptotic body or surrogate thereof bound to an apoptotic signaling molecule can be bound by Clq in apoptotic cell recognition (Paidassi et al., J. Immunol. 180:2329-2338). For example, molecules that may be useful as apoptotic signaling molecules include phosphatidyl serine, CD47, annexin-1, annexin-5, milk fat globule-EGF-factor 8 (MFG-E8), calreticulin, oxidized LDL, Fas-ligand, TNF-alpha, or the family of thrombospondins.

Thrombospondins are a family of extracellular proteins that participate in cell-to-cell and cell-to-matrix communication. They regulate phenotype during tissue genesis and repair. In addition, thrombospondin-1 (TSP-1) is expressed on apoptotic cells and is involved in their recognition by macrophages. Thrombospondin-1 is therefore another phagocytic marker that can be used to enhance phagocytosis in accordance with the invention. Macrophages recognize TSP-1 on apoptotic cells via the CD36 molecule, which is present on the surface of macrophages and may also be present on apoptotic cells. While not wishing to be bound by any theory, it is possible that CD36/TSP1 complex on the surface of an apoptotic cell may form a ligand bridging the cell to a complex consisting of alpha(v)beta 3/CD36/TSP1 on macrophages. It is possible that binding of TSP-1 to CD36 is mediated by interaction of the TSR-1 domain of TSP-1 with a conserved domain called CLESH-1 in CD36. In certain embodiments of the invention phagocytosis is enhanced by increasing the level or density of TSP-1, CD36, or a TSP-1/CD36 complex on the surface of a cell or molecule, e.g., by delivering the TSP-1, CD36, or TSP-1/CD36 complex to the cell. In certain embodiments of the invention a TSP-1/CLESH domain complex is delivered to the cell.

Alternatively or additionally, the phagocytic marker may comprise a molecule (e.g., MFG-E8, b2-glycoprotein, etc.) that serves as a bridging agent between macrophages and their targets, or a portion of such a molecule. Such markers may, for example, facilitate recognition of phosphatidyl serine by macrophages or be independently recognized. Other markers that are also known to enhance phagocytosis include protein S, the growth arrest specific gene product GAS-6, and various complement components including, but not limited to, factor B, Clq, and C3. As mentioned above, MFG-E8 is a secreted glycoprotein, which is produced by stimulated macrophages and binds specifically to apoptotic cells by recognizing aminophospholipids such as phosphatidylserine (PS). MFG-E8, when engaged by phospholipids, binds to cells via its RGD (arginine-glycine-aspartate) motif and binds particularly strongly to cells expressing alpha(v)beta(3) integrin, such as macrophages. At least two splice variants of MFG-E8 are known, of which the L variant is believed to be active for stimulating phagocytosis In certain embodiments of the invention the phagocytic marker comprises the L splice variant of MFG-E8 (MFG-E8-L). In certain embodiments of the invention the phagocytic marker comprises an N-terminal domain of MFG-E8.

Annexin I is another phagocytic marker that may be used according to the present invention. Briefly, the 37 kDa annexin 1 (Anx-1; lipocortin 1) is a glucocorticoid-regulated protein that has been implicated in the regulation of phagocytosis, cell signaling and proliferation, and is postulated to be a mediator of glucocorticoid action in inflammation and in the control of anterior pituitary hormone release. Annexin I expression is elevated in apoptotic cells and appears to play a role in bridging phosphatidylserine on apoptotic cells to phagocytes and to enhancing recognition of apoptotic cells by phagocytes such as macrophages. While not wishing to be bound by any theory, it is possible that the phosphatidylserine receptor on macrophages recognizes either annexin I or a complex containing annexin I and PS, or that annexin 1 facilitates recognition by aggregating PS into clusters. Additionally, other DC targeting studies use conjugated targeting ligands such as anti-Dec-205 and anti-CD11c to increase DC specificity.

Also provided herein is an anergy promoting agent. The agent can comprise a cytokine, such as IL-10 or TGF-β. The anergy promoting agent can promote Treg expansion, induction, of both. The anergy promoting agent can promote PD-IL, IL-10, and/or TGF-β, activity or expression, such as by promoting PD-L1-mediaed anergy.

Also provided herein is an additional agent that can be an imaging agent. The imaging agent can be linked, attached or conjugated to the apoptotic body or surrogate thereof. For example, an apoptotic body or surrogate thereof may have one or more imaging agents incorporated or conjugated to the apoptotic body or surrogate thereof. An example of an apoptotic body surrogate is a nanosphere with an imaging agent, currently commercially available is the Kodak X-sight nanospheres. Inorganic quantum-confined luminescent nanocrystals, known as quantum dots (QDs), have emerged as donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M. Angew. Chem. Int. Ed. 2003, 42 5796; Waggoner, A., Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures of highly toxic, unstable reagents. (Etienne, et al., Appl. Phys. Lett. 87, 181913, 2005).

A composition disclosed herein can comprise one or more additional agents, wherein each is attached to an apoptotic body or surrogate thereof, such as along with one or more epitopes (such as immunodominant epitopes). In another embodiment, a composition comprises a plurality of additional agents, wherein the plurality is attached to a single apoptotic body or surrogate thereof. In another embodiment, a composition comprises at least 2, 3, 4, 5, 6, 7, 8, 9, 10, 11, 12, 13, 14, 15, 16, 17, 18, 19, or 20 additional agents, wherein each is attached to an apoptotic body or surrogate thereof. In yet another embodiment, a composition comprises at least 2, 3, 4, 5, 6, 7, 8, 9, 10, 11, 12, 13, 14, 15, 16, 17, 18, 19, or 20 additional agents, wherein a plurality, such as at least 2, 3, 4, 5, 6, 7, 8, 9, 10, 11, 12, 13, 14, 15, 16, 17, 18, 19, or 20 additional agents is attached to a single apoptotic body or surrogate thereof. The additional agents can be different or the same.

In some embodiments, the additional agent is linked, attached, or conjugated to the antigen. In some instances, the additional agent, such as an apoptotic signaling molecule, and antigen, such as an antigenic peptide, are conjugated by the creation of a fusion protein. As used herein, a “fusion protein” refers to a protein formed by the fusion of at least one antigenic peptide (or a fragment or a variant thereof) to at least one molecule of an apoptotic signaling molecule (or a fragment or a variant thereof). For the creation of fusion proteins, the terms “fusion protein,” “fusion peptide,” “fusion polypeptide,” and “chimeric peptide” are used interchangeably. Suitable fragments of the antigenic peptide include any fragment of the full-length peptide that retains the function of generating the desired antigen-specific tolerance function of the present invention. Suitable fragments of the apoptotic signaling molecules include any fragment of the full-length peptide that retains the function of generating an apoptotic signal. The present application is also directed to proteins containing polypeptides at least 80%, 85%, 90%, 95%, 96%, 97%, 98% or 99% identical to the reference polypeptide sequence (e.g., the antigenic peptide or apoptotic signaling molecule or the fusion protein thereof) set forth herein, or fragments thereof. “Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide. As used herein, “variant”, refers to an antigenic peptide, apoptotic signaling molecule or fusion protein thereof differing in sequence from an antigenic peptide, apoptotic signaling molecule or fusion protein thereof of the invention, respectfully, but retaining at least one functional and/or therapeutic property thereof (e.g., trigger tolerance in an immune system of produce an apoptotic signal). The present invention is also directed to proteins which comprise, or alternatively consist of, an amino acid sequence which is at least 80%, 85%, 90%, 95%, 96%, 97%, 98%, 99% or 100% identical to, for example, the amino acid sequence of an antigenic peptide, apoptotic signaling molecule or fusion protein thereof.

The fusion protein may be created by various means. One means is by genetic fusion (i.e., the fusion protein is generated by translation of a nucleic acid sequence in which a polynucleotide encoding all or a portion or a variant of an antigenic peptide is joined in frame to a polynucleotide encoding all or a portion or a variant of an apoptotic signaling molecule. The two proteins may be fused either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. This term also refers to conservatively modified variants, polymorphic variants, alleles, mutants, subsequences, and interspecies homologs of the antigens that make up the fusion protein. The fusion protein may also be created by chemical conjugation. Protocols for generation of fusion polypeptides are well known in the art, and include various recombinant means and DNA synthesizers. Alternatively, the apoptotic signaling molecule and antigenic peptide fusion protein can also be easily created using PCR amplification and anchor primers that give rise to complementary overhangs between two consecutive gene fragments that can subsequently be annealed and reamplified to generate a chimeric gene sequence. For example, an apoptotic signaling molecule can be fused in-frame with an antigenic peptide. In the present invention, either the apoptotic signaling molecule or antigenic peptide may be the N-terminal portion of the fusion protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component to that the reading frames of the sequences are in phase. This permits translation into a single fusion protein that retains the biological activity of both component polypeptides

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46 (1985); Murphy et al., Proc Natl. Acad. Sci. USA 83:8258-8262 (1986); U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

### Method of Use

Also provided herein is a method of administering a composition as disclosed herein. The method comprises regulating an immune response in a subject. Methods of immunoregulation include those that suppress and/or inhibit an innate immune response, including, but not limited to, an immune response stimulated by immunostimulatory polypeptides, such as myelin basic protein. In one embodiment, the method induces tolerance to a specific antigen. The method can comprise reducing hypersensitivity to an antigen in a subject. The antigen can be an allergen, therapeutic, or tissue or cell to be transplanted to a subject that would otherwise induce T-cell receptor-mediated stimulation. In another embodiment, the method induces anergy, such as PD-L1 mediate anergy. The method can also comprise inducing IL-10 and/or PD-L1 expression, activity, or both. The method can also comprise targeting or delivering an antigen, or an immunodominant epitope, the splenic marginal zone.

In one embodiment, a composition comprising an apoptotic body, or apoptotic body surrogate, and an epitope of an antigen is administered to a subject, and antigen-specific tolerance is induced in the subject. In another embodiment, a composition comprising an apoptotic body, or apoptotic body surrogate, and a plurality of epitopes of one or more antigens is administered to a subject, and tolerance to at least one or more of the antigens is induced in the subject. In one embodiment, the plurality can comprise a subset of immunodominant epitopes or all of the epitopes in the plurality are immunodominant epitopes. The composition can be administered or delivered to a subject orally, nasally, intravenously, intramuscularly, parenterally, or ocularly. In preferred embodiments, the composition is administered intravenously.

The subject can be suffering from or at risk of a condition and the antigen is suspected or known to cause the condition. For example, the antigen may act as an allergen that would otherwise induce T-cell receptor-mediated stimulation in the subject. Administration of the composition can be prior to concurrent, or subsequent to with onset of said condition. In some embodiments, the composition prevents relapse of the condition. In other embodiments, the invention relates to uses of the compositions disclosed herein to inhibit ongoing of the condition. In some embodiments, the invention relates to ameliorating the condition. By ameliorating a condition is meant to include treating, preventing or suppressing the condition in a subject.

The subject can be any organism with an immune response. For example, the subject can be a mammal, such as a human, monkey, dog, cat, rabbit or rodent.

Certain embodiments relate to the subject being primed with a composition of the present invention (i.e., apoptotic body or surrogate thereof with an antigen), to prime a subject for immune tolerance. These embodiments generally involve a plurality of administrations of an immune tolerance inducing composition. For example, at least 2, 3, 4, 5, 6 or more administrations are performed during priming in order to achieve a long-lasting result, although the subject may show manifestations of tolerance early in the course of treatment. In one embodiment, each dose is given as a bolus administration. In another embodiment, sustained formulations capable of mucosal release are used. Where multiple administrations are performed, the time between administrations is generally between 1 day and 3 weeks, and typically between about 3 days and 2 weeks.

Other embodiments relate to boosting or extending the persistence of a previously established immune tolerance. These embodiments can involve one administration or a short course of treatment (such as at least 2, 3, 4, 5, 6, or more administrations) at a time when the established tolerance is declining or at risk of declining. Boosting can be performed from 1 month to 1 year, such as 2 to 6 months after priming with an immune tolerance inducing composition or a previous boost. This invention also includes embodiments that involve regular maintenance of tolerance on a schedule of administrations that occur semiweekly, weekly, biweekly, monthly, yearly, or on any other regular schedule.

In certain embodiments, the subject is at risk for or has a condition that comprises a hypersensitive reaction to a substance, such as an allergen. For example, the method can relate to treatment of pathological conditions relating to an unwanted hypersensitivity. The hypersensitivity can be any one of types I, II, III, and IV. The frequency of administration will typically correspond with the timing of allergen exposure. Suitable animal models are known in the art (for example, Gundel et al., Am. Rev. Respir Dis. 146:369, 1992; Wada et al., J. Med. Chem. 39, 2055, 1996; and WO 96/35418).

The subject may have never been exposed to an allergen that the subject is allergic to, such as never being exposed to a food or therapeutic to which the subject is allergic to. In another embodiment, the subject has been previously exposed to the allergen and had an adverse or hypersensitive reaction, such as being exposed to a food to which the subject has had an adverse reaction to. In one embodiment, the method comprises reducing the risk of having a hypersensitive reaction to the allergen or inducing tolerance to the allergen thereby reducing the hypersensitivity response to the allergen in the subject.

For example, the method can comprise administering to a subject a composition comprising an apoptotic body or surrogate thereof, and an antigen from an allergen in which the subject is at risk or would have a hypersensitive response to. Administration reduces or eliminates an adverse reaction to any subsequent contact the subject has with the allergen. The composition can be administered prior to, concurrent with, or subsequent to the subject's exposure to the allergen, such as prior to, concurrent with, or subsequent to a subject's contact with a therapeutic, vaccine, or food to which the subject may be at risk for, or have had, an adverse reaction to. Administering to the subject the composition comprising an apoptotic body or surrogate thereof, and an antigen of the allergen can reduce or eliminates any hypersensitivity response or adverse reaction the subject would have without being administered the composition.

In another embodiment, the method comprises administering to a subject a composition comprising an apoptotic body or surrogate thereof, and an antigen from a tissue or cell to be transplanted or that has been transplanted to a subject. The subject may be receiving a transplant or has received a transplant. The subject may have previously rejected a transplant. Alternatively, the subject may not have in experienced a transplant rejection. Administration of the composition can reduce the risk of transplant rejection, such as for a subject to receive a transplant. In one embodiment, administration can suppress transplant rejection or induce tolerance of the transplanted tissue or cell in the subject thereby reducing the risk of transplant rejection in the subject. Administering of the composition can be performed prior to, concurrent with, or subsequent to transplantation of the tissue or cell.

Transplantation can refer to the transfer of a tissue sample or graft from a donor individual to a recipient individual, such as frequently performed on human recipients who need the tissue in order to restore a physiological function provided by the tissue. Tissues that are transplanted include (but are not limited to) whole organs such as kidney liver, heart, lung; organ components such as skin grafts and the cornea of the eye; and cell suspensions such as bone marrow cells and cultures of cells selected and expanded from bone marrow or circulating blood, and whole blood transfusions.

A serious potential complication of any transplantation can ensue from antigenic differences between the host recipient and the engrafted tissue. Depending on the nature and degree of the difference, there may be a risk of an immunological assault of the graft by the host, or of the host by the graft, or both, may occur. The extent of the risk is determined by following the response pattern in a population of similarly treated subjects with a similar phenotype, and correlating the various possible contributing factors according to well accepted clinical procedures. The immunological assault may be the result of a preexisting immunological response (such as preformed antibody), or one that is initiated about the time of transplantation (such as the generation of T_{H} cells). Antibody, T_{H} cells, or T_{C} cells may be involved in any combination with each other and with various effector molecules and cells.

A composition and method disclosed herein can provide materials and procedures that permit transplantation to be conducted according to standard surgical procedures, but with decreased risk of an adverse immunological reaction to the recipient of the transplant. The procedure can involve tolerizing the recipient to the tissues of the donor, or vice versa, or both. The tolerizing can be performed by administering a target antigen expressed in the transplanted tissue, such as comprising an immunodominant epitope of the target antigen, or a bystander antigen, along with an apoptotic body or surrogate thereof. The target antigen may be, for example, allogeneic cell extracts. The graft may be a complex structure of many different cell types, and any one or more of the cell types transplanted into the individual may pose a risk for which the procedures of this invention are appropriate. For example, endothelial cell antigens complicate renal transplants, and passenger lymphocytes complicate hepatic transplants.

In another embodiment, the risk of host versus graft disease, leading to rejection of the tissue graft by the recipient is reduced. The treatment may be performed to prevent or reduce the effort of a hyperacute, acute, or chronic rejection response. Treatment can be initiated sufficiently far in advance of the transplant so that tolerance is in place when the graft is installed; but where this in not possible, treatment can be initiated simultaneously with or following the transplant. Regardless of the time of initiation, in one embodiment, treatment can continue at regular intervals for at least the first month following transplant. Follow-up doses may not be required if a sufficient accommodation of the graft occurs, but can be resumed if there is any evidence of rejection or inflammation of the graft. The tolerization procedures disclosed herein may be combined with other forms of immunosuppression to achieve an even lower level of risk.

In another embodiment, decreasing the risk of graft versus host disease is achieved by tolerizing a living donor against a target antigen of the future graft recipient before the transplantation occurs. Once tolerance is achieved, the cells or tissue of the donor are harvested and the transplant is performed.

The subject may be at risk or have a condition associated with unwanted immune activation, such as an autoimmune disease of inflammatory disease. Autoimmune diseases can be divided in two broad categories: organ-specific and systemic. Autoimmune diseases include, without limitation, rheumatoid arthritis (RA), systemic lupus erythematosus (SLE), type I diabetes mellitus, type II diabetes mellitus, multiple sclerosis (MS), neuromyelitis optica, immune-mediated infertility such as premature ovarian failure, scleroderma, Sjogren's disease, vitiligo, alopecia (baldness), polyglandular, Grave's disease, hypothyroidism, polymyositis, pemphigus vulgaris, pemphigus foliaceus, inflammatory bowel disease including Crohn's disease and ulcerative colitis, autoimmune hepatitis including that associated with hepatitis B virus (HBV) and hepatitis C virus (IICV), hypopituitarism, graft-versus-host disease (GvHD), myocarditis, Addison's disease, autoimmune skin diseases, uveitis, pernicious anemia, and hypoparathyroidism.

Autoimmune diseases may also include, without limitation, Hashimoto's thyroiditis, Type I and Type II autoimmune polyglandular syndromes, paraneoplastic pemphigus, bullus pemphigoid, dermatitis herpetiformis, linear IgA disease, epidermolysis bullosa acquisita, erythema nodosa, pemphigoid gestationis, cicatricial pemphigoid, mixed essential cryoglobulinemia, chronic bullous disease of childhood, hemolytic anemia, thrombocytopenic purpura, Goodpasture's syndrome, autoimmune neutropenia, myasthenia gravis, Eaton-Lambert myasthenic syndrome, stiff-man syndrome, acute disseminated encephalomyelitis, Guillain-Barre syndrome, chronic inflammatory demyelinating polyradiculoneuropathy, multifocal motor neuropathy with conduction block, chronic neuropathy with monoclonal gammopathy, opsonoclonus-myoclonus syndrome, cerebellar degeneration, encephalomyelitis, retinopathy, primary biliary sclerosis, sclerosing cholangitis, gluten-sensitive enteropathy, ankylosing spondylitis, reactive arthritides, polymyositis/dermatomyositis, mixed connective tissue disease, Bechet's syndrome, psoriasis, polyarteritis nodosa, allergic anguitis and granulomatosis (Churg-Strauss disease), polyangiitis overlap syndrome, hypersensitivity vasculitis, Wegener's granulomatosis, temporal arteritis, Takayasu's arteritis, Kawasaki's disease, isolated vasculitis of the central nervous system, thromboangiutis obliterans, sarcoidosis, glomerulonephritis, and cryopathies. These conditions are well known in the medical arts and are described, for example, in Harrison's Principles of Internal Medicine, 14^{th} ed., Fauci A S et al., eds., New York: McGraw-Hill, 1998.

In one embodiment, the method comprises inducing tolerance to an autoantigen for the treatment of an autoimmune disease by administering the antigen for which tolerance is desired. For example, autoantibodies directed against the myelin basic protein (MBP) are observed in subjects with multiple sclerosis, and, accordingly, MBP antigenic peptides or proteins may be used in the invention to be delivered using the compositions of the present invention to treat and prevent multiple sclerosis. For another example, autoantibodies directed against the water channel aquaporin 4 are observed in subjects with neuromyelitis optica, and, accordingly, aquaporin 4 antigenic peptides or proteins may be used in the invention to be delivered using the compositions of the present invention to treat and prevent neuromyelitis optica. In one embodiment, one or more immunodominant epitopes of the antigenic peptides are administered.

In some embodiments, the invention relates to preventing the relapse of disease. For example, an unwanted immune response can occur at one region of an antigen (such as an antigenic determinant or immunodominant epitope). Relapse of a disease associated with an unwanted immune response can occur by having an immune response attack at a different region of the antigen. T-cell responses in some immune response disorders, including MS and other Th1/17-mediated autoimmune diseases, can be dynamic and evolve during the course of relapsing-remitting and/or chronic-progressive disease. The dynamic nature of the T-cell repertoire has implications for treatment of certain diseases, since the target may change as the disease progresses. Previously, pre-existing knowledge of the pattern of responses was necessary to predict the progression of disease. The present invention provides compositions that can prevent the effect of dynamic changing disease, a function of “epitope spreading.” One model for relapse is an immune reaction to proteolipid protein (PLP) as a model for multiple sclerosis (MS). Initial immune response can occur by a response to PLP₁₃₉₋₁₅₁. Subsequent disease onset can occur by a relapse immune response to PLP₁₇₈₋₁₉₁. Compositions of the present invention have been shown to prevent relapse of disease using the PLP model.

Animal models for the study of autoimmune disease are known in the art. For example, animal models which appear most similar to human autoimmune disease include animal strains which spontaneously develop a high incidence of the particular disease. Examples of such models include, but are not limited to, the nonobeses diabetic (NOD) mouse, which develops a disease similar to type I diabetes, and lupus-like disease prone animals, such as New Zealand hybrid, MRL-Fas^{lpr} and BXSB mice. Animal models in which an autoimmune disease has been induced include, but are not limited to, experimental autoimmune encephalomyelitis (EAE), which is a model for multiple sclerosis, collagen-induced arthritis (CIA), which is a model for rheumatoid arthritis, and experimental autoimmune uveitis (EAU), which is a model for uveitis. Animal models for autoimmune disease have also been created by genetic manipulation and include, for example, IL-2/IL-10 knockout mice for inflammatory bowel disease, Fas or Fas ligand knockout for SLE, and IL-1 receptor antagonist knockout for rheumatoid arthritis.

In a further example, sensitization of a subject to an industrial pollutant or chemical, such as may be encountered on-the-job, presents a hazard of an immune response. Prior tolerance of the individual's immune system to the chemical, in particular in the form of the chemical reacted with the individual's endogenous proteins, may be desirable to prevent the later occupational development of an immune response. In one embodiment, a subject can be administered a composition comprising an apoptotic body or surrogate thereof with such a chemical.

The compositions disclosed herein can be used to deliver the antigen to the spleen of a subject. In one embodiment, a method for delivering an antigen to the spleen, such as specifically the splenic marginal zone, can comprise administering a composition comprising an apoptotic body or surrogate thereof and the antigen to a subject. The apoptotic body or surrogate thereof, along with the antigen, can be recognized by a macrophage scavenger receptor. The macrophage can be in the spleen, such as specifically in the splenic marginal zone. The macrophage scavenger receptor can uptake the apoptotic body or surrogate thereof and the antigen. In one embodiment, the apoptotic body or surrogate thereof is cleared from a spleen or splenic zone within 72, 48, 24, 12, 11, 10, 9, 8, 7, 6, 5, 4, 3, 2, or 1 hour(s). The macrophage scavenger receptor can be LOX-1, SRBI, SRBII, or MARCO. Scavenger receptor, such as MARCO, may function through its ability to uptake particles, e.g., Ag-linked particles and assist on macrophage antigen presentation or antigen transfer to local dendritic cells. MARCO or other scavenger receptors may also inhibit inflammatory responses by preventing dendritic cell migration or by other unknown anti-inflammatory mechanisms.

The methods disclosed herein can also comprise determining or identifying a specific antigen to which a subject is hypersensitive to, or at risk of having a hypersensitive response, autoimmune condition, or inflammatory condition. The specific antigen may be identified as being associated with a condition for a first subject but not a second subject with the same condition, for example, a different antigen may be identified for the second subject.

Determination or identification of the antigen can be used to select one or more specific antigens or epitopes, such as immunodominant epitopes, of the one or more specific antigens to be used in a composition with an apoptotic body or surrogate thereof. The composition can be administered to the subject to induce tolerance to the one or more specific antigens.

In one embodiment, the method comprises determining from personalized information from the subject the specific antigen. The personalized antigen may be obtained from the subject or a third party, such as a physician or health care professional of the subject, a guardian or caretaker of the subject, or indirectly from the subject (such as a subject consenting to a genetic testing company releasing the results for determining an antigen to which the subject is hypersensitive to).

The personalized information can comprise the medical history, family history, genotype information, or any combination thereof of the subject. For example, the personalized information can comprise allergic reaction information, autoimmune disorder records, or inflammatory disorder records of the subject or family members of the subject. The personalized information can comprise surveys or questionnaires with questions asking about the subject's diet, exercise habits, allergic reactions, pre-existing conditions, work and living environment, daily activities, or any combination thereof. The personalized information can comprise laboratory results.

The genotype information can comprise information such as the subject DNA sequence, such as the subject's complete genome or a portion thereof. The genotype information can comprise information about any genetic mutations, deletions, insertions, or polymorphisms the subject may have. The genotype information can comprise information about any variants or variations, such as copy number variants. The method of determining from personalized information one or more specific antigens, to which a subject may have a hypersensitive response to, can also comprise generating the genotype. Alternatively, the genotype information is generated by a third party.

In another embodiment, the method of determining or identifying a specific antigen to which a subject is hypersensitive to, or at risk of having a hypersensitive response, autoimmune condition, or inflammatory condition comprises determining from a pool of immune cells from a subject an antigen to which said subject is hypersensitive. This can be done in combination with determining from personalized information of the subject, or in isolation. In one embodiment, the method comprises obtaining a pool of immune cells from a subject and determining from the pool one or more antigens to which the subject is hypersensitive to. The method can further comprise administering a composition comprising an apoptotic body or surrogate thereof and an epitope, such as an immunodominant epitope, of the one or more antigens to the subject. Tolerance specific to the antigen can be induced in the subject.

The immune cells may comprise lymphocytes, such as T-cells. The immune cells may be a mixed cell population or a population consisting of essentially one type of immune cells, such as a T-cell population. In one embodiment, determining the specific antigen comprises subjecting the immune cells to a variety of antigens and identifying a T-cell response to an antigen, thereby determining an antigen to which said subject is hypersensitive to. T-cell response can be performed by using any assay known in the art. In one embodiment, the T-cell response is assayed by determining T-cell proliferation of cytokine secretion. The T-cell response can be assayed by flow cytometry.

### Evaluating Tolerance

Also provided herein is a method of evaluating tolerance induction or hypersensitivity reduction using a composition or method of inducing tolerance as disclosed here. For example, a specific antigen, epitope, immunodominant epitope or combination thereof can be tested for their ability to promote tolerance by conducting experiments with isolated cells or in animal models.

A proxy for tolerogenic activity is the ability of an intact antigen or fragment to stimulate the production of an appropriate cytokine at the target site. The immunoregulatory cytokine released by T suppressor cells at the target site is thought to be TGF-β (Miller et al., Proc. Natl. Acad. Sci. USA 89:421, 1992). Other factors that may be produced during tolerance are the cytokines IL4 and IL-10, and the mediator PGE. In contrast, lymphocytes in tissues undergoing active immune destruction secrete cytokines such as IL-1, IL-2, IL-6, and γ-IFN. Hence, the efficacy of a candidate inducing antigen can be evaluation by measuring its ability to stimulate the appropriate type of cytokines.

A rapid screening test for tolerogenic epitopes of the inducing antigen, effective mucosal binding components, effective combinations, or effective modes and schedules of mucosal administration can be conducted using syngeneic animals as donors for in vitro cell assays. In one embodiment, animals are treated at a mucosal surface with the test composition, and at some time are challenged with parenteral administration of the target antigen in complete Freund's adjuvant. Spleen cells are isolated, and cultured in vitro in the presence of the target antigen at a concentration of about 50 μg/mL. Target antigen can be substituted with candidate proteins or sub-fragments to map the location of tolerogenic epitopes. Cytokine secretion into the medium can be quantified by standard immunoassay.

The ability of the cells to suppress the activity of other cells can be determined using cells isolated from an animal immunized with the target antigen, or by creating a cell line responsive to the target antigen (Ben-Nun et al., Eur. J. Immunol., 11:195, 1981). In one variation of this experiment, the suppressor cell population is mildly irradiated (about 1000 to 1250 rads) to prevent proliferation, the suppressors are co-cultured with the responder cells, and then tritiated thymidine incorporation (or MTT) is used to quantify the proliferative activity of the responders. In another variation, the suppressor cell population and the responder cell population are cultured in the upper and lower levels of a dual chamber transwell culture system (Costar, Cambridge Mass.), which permits the populations to coincubate within 1 mm of each other, separated by a polycarbonate membrane (WO 93/16724). In this approach, irradiation of the suppressor cell population is unnecessary, since the proliferative activity of the responders can be measured separately.

In other embodiments, methods known in the art for diagnosing MS can be used for determining the effectiveness of a composition disclosed herein. For example, a subject with MS administered a composition disclosed herein can have magnetic resonance imaging (MRI), visual evoked potentials (VEP), cerebrospinal fluid analysis, or any combination thereof performed to determine whether inflammation or CNS damage has been increased, decreased, or relatively unchanged as compared to prior administration. Increased damage can be used as an indication that the composition is ineffective, or does not promote tolerance. In some embodiments, unchanged CNS damage or inflammation can also be used as an indication that the composition is ineffective, or does not promote tolerance. However, in other embodiments, such as when inflammation or CNS damage is expected to increase in the subject, unchanged CNS damage or inflammation can also be used as an indication that the composition is effective, or does promote tolerance. Decreased inflammation or CNS damage can be used as an indication that the composition is effective, or does promote tolerance as well.

The effectiveness of compositions and modes of administration for treatment of specific disease can also be elaborated in a corresponding animal disease model. The ability of the treatment to diminish or delay the symptomatology of the disease is monitored at the level of circulating biochemical and immunological hallmarks of the disease, immunohistology of the affected tissue, and gross clinical features as appropriate for the model being employed. Non-limiting examples of animal models that can be used for testing are included herein. In one embodiment, the animal model is an experimental allergic encephalomyelitis (EAE) mouse model. The EAE mouse model can be a relapsing EAE (R-EAE) mouse model. In one embodiment, the methods for evaluating tolerance disclosed herein can be performed on a mouse, such as an EAE mouse model.

The present invention also contemplates modulation of tolerance by modulating TH1 response, TH2 response, TH17 response, or a combination of these responses. Modulating TH1 response encompasses changing expression of, e.g., interferon-gamma. Modulating TH2 response encompasses changing expression of e.g., any combination of IL-4, IL-5, IL-10, and IL-13. Typically an increase (decrease) in TH2 response will comprise an increase (decrease) in expression of at least one of IL-4, IL-5, IL-10, or IL-13; more typically an increase (decrease) in TH2 response will comprise an increase in expression of at least two of IL-4, IL-5, IL-10, or IL-13, most typically an increase (decrease) in TH2 response will comprise an increase in at least three of IL-4, IL-5, IL-10, or IL-13, while ideally an increase (decrease) in TH2 response will comprise an increase (decrease) in expression of all of IL-4, IL-5, IL-10, or IL-13. Modulating TH17 encompasses changing expression of, e.g., TGF-beta, IL-6, IL-21 and IL23, and effects levels of IL-17, IL-21 and IL-22.

Tolerance to autoantigens and autoimmune disease can be achieved by a variety of mechanisms including negative selection of self-reactive T cells in the thymus and mechanisms of peripheral tolerance for those autoreactive T cells that escape thymic deletion and are found in the periphery. Examples of mechanisms that provide peripheral T cell tolerance include “ignorance” of self-antigens, anergy or unresponsiveness to autoantigen, cytokine immune deviation, and activation-induced cell death of self-reactive T cells. In addition, regulatory T cells have been shown to be involved in mediating peripheral tolerance. See, for example Walker et al., (2002) Nat. Rev. Immunol, 2:11-19; Shevach et al., (2001) Immunol. Rev. 182:58-67. In some situations, peripheral tolerance to an autoantigen is lost (or broken) and an autoimmune response ensues. For example, in an animal model for EAE, activation of antigen presenting cells (APCs) through TLR innate immune receptors was shown to break self-tolerance and result in the induction of EAE (Waldner et al., (2004) J. Clin, Invest. 113:990-997).

In some embodiments, tolerance induction can be evaluated by analyzing whether antigen presentation and/or TLR7/8, TLR9, and/or TLR7/8/9 dependent cell stimulation is increased or reduced as compared to a control subject (i.e., a subject not administered a composition disclosed here). Administration of a composition disclosed herein can result in antigen presentation by DCs or APCs while suppressing the TLR7/8, TLR9, and/or TLR7/8/9 dependent cell responses associated with immunostimulatory polynucleotides. Such suppression may include decreased levels of one or more TLR-associated cytokines.

### Pharmaceutical Compositions

The composition disclosed herein can be a pharmaceutical composition. In one embodiment, the composition of an apoptotic body or surrogate thereof and an antigen can be administered in combination with other pharmaceutical agents, as described herein, and can be combined with a physiologically acceptable carrier thereof (and as such the invention includes these compositions).

Compositions can be prepared for administration to an individual in need thereof, particularly human subjects having an unwanted immune response. The preparation of compositions and their use is conducted in accordance with generally acceptance procedures for the preparation of pharmaceutical compositions.

Procedures for preparing pharmaceutical compositions are described in Remington's Pharmaceutical Sciences, E. W. Martin ed., Mack Publishing Co., Pa. The composition (whether given separately or together) can be optionally combined with other active components, carriers and excipients, and stabilizers. Additional active components of interest are agents that enhance the tolerogenic effect of the combination. An example of an additional active component is a cytokine, such as IL-10, IL-4, or any others described herein or found suitable for inducing immune tolerance. Pharmaceutical compositions can be supplied in unit dosage form suitable for administration of a precise amount.

The effective amounts and method of administration of the present invention for modulation of an immune response can vary based on the individual, what condition is to be treated and other factors evident to one skilled in the art. Factors to be considered include route of administration and the number of doses to be administered. Such factors are known in the art and it is well within the skill of those in the art to make such determinations without undue experimentation. A suitable dosage range is one that provides the desired regulation of immune response (e.g., suppression of IFN-α or other cytokine production). Useful dosage ranges of the carrier, given in amounts of carrier delivered, may be, for example, from about any of the following: 0.5 to 10 mg/kg, 1 to 9 mg/kg, 2 to 8 mg/kg, 3 to 7 mg/kg, 4 to 6 mg/kg, 5 mg/kg, 1 to 10 mg/kg, 5 to 10 mg/kg. Alternatively, the dosage can be administered based on the number of particles. For example, useful dosages of the carrier, given in amounts of carrier delivered, may be, for example, from about any of the following: greater than 10⁶, 10⁷, 10⁸, 10⁹, or 10¹⁰ particles per dose, or from 1×10⁷ to 1×10⁹ particles per dose, or from 1×10⁸ to 1×10⁹ particles per dose, or from 2×10⁹ to 5×10⁹ particles per dose. The absolute amount given to each patient depends on pharmacological properties such as bioavailability, clearance rate and route of administration. Details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa., USA, which is hereby incorporated by reference in its entirety.

The effective amount and method of administration of the particular formulation can vary based on the individual patient, desired result and/or type of disorder, the stage of the disease and other factors evident to one skilled in the art. The route(s) of administration useful in a particular application are apparent to one of skill in the art. Routes of administration include but are not limited to topical, dermal, transdermal, transmucosal, epidermal, parenteral, gastrointestinal, and naso-pharyngeal and pulmonary, including transbronchial and transalveolar. In one particular embodiment, administration is performed intravenously. A suitable dosage range can be one that provides sufficient tissue concentration, such as of about 1-50 μM, as measured by blood levels. However, the absolute amount given to each subject depends on pharmacological properties such as bioavailability, clearance rate and route of administration.

The present invention also provides carrier formulations suitable for topical application including, but not limited to, physiologically acceptable implants, ointments, creams, rinses and gels. Exemplary routes of dermal administration are those which are least invasive such as transdermal transmission, epidermal administration and subcutaneous injection.

Transdermal administration is accomplished by application of a cream, rinse, gel, etc., capable of allowing the carrier to penetrate the skin and enter the blood stream. Compositions suitable for transdermal administration include, but are not limited to, pharmaceutically acceptable suspensions, oils, creams and ointments applied directly to the skin or incorporated into a protective carrier such as a transdermal device (so-called “patch”). Examples of suitable creams, ointments, etc., can be found, for instance, in the Physician's Desk Reference. Transdermal transmission may also be accomplished by iontophoresis, for example using commercially available patches which deliver their product continuously through unbroken skin for periods of several days or more. Use of this method allows for controlled transmission of pharmaceutical compositions in relatively great concentrations, permits infusion of combination drugs and allows for contemporaneous use of an absorption promoter.

Parenteral routes of administration include but are not limited to electrical (iontophoresis) or direct injection such as direct injection into a central venous line, intravenous, intramuscular, intraperitoneal, intradermal, or subcutaneous injection. Formulations of carrier suitable for parenteral administration are generally formulated in USP water or water for injection and may further comprise pH buffers, salts bulking agents, preservatives, and other pharmaceutically acceptable excipients. Immunoregulatory polynucleotide for parenteral injection may be formulated in pharmaceutically acceptable sterile isotonic solutions such as saline and phosphate buffered saline for injection.

Gastrointestinal routes of administration include, but are not limited to, ingestion and rectal routes and can include the use of, for example, pharmaceutically acceptable powders, pills or liquids for ingestion and suppositories for rectal administration.

Naso-pharyngeal and pulmonary administration include are accomplished by inhalation, and include delivery routes such as intranasal, transbronchial and transalveolar routes. The invention includes formulations of carrier suitable for administration by inhalation including, but not limited to, liquid suspensions for forming aerosols as well as powder forms for dry powder inhalation delivery systems. Devices suitable for administration by inhalation of carrier formulations include, but are not limited to, atomizers, vaporizers, nebulizers, and dry powder inhalation delivery devices.

As is well known in the art, solutions suspensions used for the routes of administration described herein can include any one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

As is well known in the art, pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition must be sterile and can be fluid to the extent that easy syringability exists. It can be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Some embodiments include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

As is well known in the art, sterile injectable solutions can be prepared by incorporating the active compound(s) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Certain embodiments of the present invention relate to kits and reagents in which one or more component is provided in a separate container, optionally with written instructions, for assembly of a pharmaceutical composition by the subject or the administering health professional.

### EXAMPLES

### Example 1. Tolerance Induction Requires i.v. Administration of Ag-SP and is Spleen Dependent

The role of the spleen and route of administration in peptide-coupled splenocytes (or antigen-splenocyte, “Ag-SP”) tolerance induction was examined. First the route of Ag-SP administration was investigated and the results are depicted in FIG. 1A. Synthetic peptides myelin oligodendrocyte glycoprotein (MOG)₃₅₋₅₅ (MEVGWYRSPFSRVVHLYRNGK), proteolipid protein (PLP)₁₃₉₋₁₅₁ (HSLGKWLGHPDKF), and OVA₃₂₃₋₃₃₉ (ISQAVHAAHAEINEAGR), were purchased from Genemed Synthesis. PLP₁₇₈₋₁₉₁ (NTWTTCQSIAFPSK) was purchased from Peptides International. Female SJL/J mice, 5-7 wk old, were purchased from Harlan Laboratories and were housed under specific pathogen-free conditions in the Northwestern University Center for Comparative Medicine and maintained according to protocols approved by the Northwestern University Institutional Animal Care and Use Committee.

Spleens were removed from naive female mice, and the red blood cells were lysed. The splenocytes were incubated with ECDI (150 mg/3.2×10⁸ cells; Calbiochem) and peptide (1 mg/ml) on ice, shaking for 1 h. The coupled cells (Ag-SP) were washed three times and filtered through a 70-μM cell strainer to remove cell clumps. The Ag-SP were resuspended at 250×10⁶ cells/ml in PBS.

SJL/J mice were tolerized with 5×10⁷ sham OVA₃₂₃₋₃₃₁-SP given intravenously (i.v.) (OVA323-SP i.v.) or with 5×10⁷ PLP₁₃₉₋₁₅₁-SP given i.v. (PLP139-SP i.v.), subcutaneously (s.c.) (PLP139-SP s.c.), or intraperitoneally (i.p.) (PLP139-SP i.p.). Five days later, the mice were immunized with 50 μg PLP₁₃₉₋₁₅₁/CFA. Mice were primed with an emulsion containing 1 mg/ml peptide and CFA containing 2 mg/ml Mycobacterium tuberculosis H37Ra (Difco). A 100 μl vol of emulsion was injected s.c. among three sites on the flank of each mouse. Mice were monitored for clinical EAE for 20 d postpriming (FIG. 1A). Individual animals were observed daily, and clinical scores were assessed in a blinded fashion on a 0-5 scale, as follows: 0, no abnormality; 1, limp tail or hind limb weakness; 2, limp tail and hind limb weakness; 3 hind limb paralysis; 4, hind limb paralysis and forelimb weakness; and 5, moribund. The data are reported as the mean daily clinical score. Paralyzed animals were afforded easier access to food and water.

I.v. administration prevented disease induction, but i.p. administration was ineffective, as mice that received PLP₁₃₉₋₁₅₁-SP i.p. developed EAE clinical scores that were similar to control mice treated i.v. with Ag-SP coupled with an irrelevant OVA₃₂₃₋₃₃₉ peptide (sham tolerized). In contrast, s.c. administration of PLP₁₃₉₋₁₅₁-SP acted synergistically with immunization, in that treated mice displayed significantly higher disease scores than sham-tolerized controls. The importance of i.v. administration of Ag-SP for tolerance induction could be related to the requirement for i.v. delivery of Ag to organs such as the spleen and liver, which have been associated with tolerance induction.

### Example 2. Scavenger Receptor Response to Ag-SP Infusion

Scavenger receptor response to Ag-SP infusion was monitored. Tolerance was induced by i.v. injection of chemically treated Ag-SP, as described in Example 1. Spleens were removed from naive female mice, and the red blood cells were lysed. The Ag-SP were labeled with green fluorescence membrane labeling agent PKH76 prior to ECDI fixation. Spleen cell membranes were stained with PKH76 (green) or with PKH26 (red) (Sigma-Aldrich) dye, according to the manufacture's instructions, before ECDI fixation. CFSE labeling was performed, as described in the manufacture's instructions (Cayman Chemical). The splenocytes were incubated with ECDI (150 mg/3.2×10⁸ cells; Calbiochem) and peptide (1 mg/ml) on ice, shaking for 1 h. The coupled cells were washed three times and filtered through a 70-μM cell strainer to remove cell clumps. The Ag-SP were resuspended at 250×10⁶ cells/ml in PBS. SJL/J mice were infused with 5×10⁷ PKH76-labeled PLP₁₃₉₋₁₅₁-SP i.v. Spleens were harvested for immune-histochemical staining 3 h later.

Antibodies used for immunohistochemistry on spleen sections included rabbit polyclonal anti-LOX-1 (Abcam), rabbit polyclonal SRBI (Abcam) and SRBII (Abnova), and anti-CD68. Polyclonal anti-rabbit, hamster IgG, rat IgG2a, or rat IgG1 Abs were used, respectively, as controls (Vector Laboratories, Biolegend, BD Pharmingen). Spleens removed from mice were infused with PKH76-labeled Ag-SP and fixed in paraformaldehyde for 30 min to 3 h at 4° C. in the dark. Spleens were then frozen in OCT. The blocks were stored at −80° C. in plastic bags to prevent dehydration. Six-micrometer-thick cross-sections were cut on a Reichert-Jung Cryocut CM1850 cryotome (Leica) mounted on Superfrost Plus electrostatically charged slides (Fisher), air dried, and stored at −80° C. Slides were stained using Tyramide Signal Amplification Direct kit (NEN), according to the manufacturer's instructions. Nonspecific staining was blocked using either anti-CD16/CD32 (FcIII/IIR, 2.4G2; BD Pharmingen) or 10% horse serum, as well as avidin/biotin blocking kit (Vector Laboratories), in addition to the blocking reagent provide by the Tyr-amide Signal Amplification kit (NEN). Sections were then stained with primary and secondary antibody mixtures). Sections were coverslipped with Vectashield mounting medium with DAPI (Vector Laboratories). Slides were examined and images were acquired using a Lica DM5000B fluorescent microscope and Advanced SPOT software. At least eight serial sections from each sample per group were analyzed at original magnification, X20, X40, and X100.

As shown in FIG. 1 B, Ag-SP accumulated in the marginal zone (MZ) of the spleen. Expression of scavenger receptors LOX-1 (FIG. 1B), SRBI (FIG. 1C), and CD68 (FIG. 1D) was unaffected, but SRBII was upregulated (FIG. 1F) compared with noninfused mice (FIG. 1 E). Some colocalization of SRBII and PKH76 was observed (white arrowhead in FIG. 1H), but not in isotype controls (FIG. 1G).

Spleen is thought to be a major site for the removal of circulating senescing erythrocytes and apoptotic hematopoietic cells The effects of Ag-SP administration on expression of certain scavenger receptors known to play a role in the removal of apoptotic cellular debris was examined. Although scavenger receptors LOX-1, SRBI, and CD68 were not affected by the accumulation of Ag-SP in the spleen at the time points examined, SRBII was upregulated within 3 h after Ag-SP infusion (FIGS. 1 B-H).

### Example 3. Tolerance Induction is Spleen Dependent

The requirement of spleen for Ag-SP tolerance was tested. Sham-splenectomized (Sham Splx) or splenectomized (Splx) SJL mice were tolerized with OVA₃₂₃₋₃₃₁-SP or PLP₁₃₉₋₁₅₁-SP on day −7 as described in Example 1 and primed s.c. with 50 μg PLP₁₃₉₋₁₅₁/CFA on day 0, and delayed-type hypersensitivity (DTH) responses to PLP₁₃₉₋₁₅₁ were determined 8 d later (FIG. 1I). DTH was performed via ear challenge with 10 μg peptide, as described in Turley and Miller, 2007, J. Immunol. 178: 2212-2220. For proliferation assays, draining lymph nodes (axillary, brachial, and inguinal) were harvested from naive mice or primed mice 8 days following disease induction, counted, and cultured in 96-well microtiter plates at a density of 5×10⁵ cells/wells in a total volume of 200 μl HL-1 medium (Bio Whittaker, 1% penicillin/streptavidin and 1% glutamine). Cells were cultured at 37° C. with medium alone or with difference concentrations of peptide Ag for 72 h. During the last 24 h, cultures were pulsed with 1 μCi/well [³H]TdR, and uptake was detected using a Topcount microplate scintillation counter; results are expressed as mean of triplicate cultures. Asterisks denote a significant reduction in DTH responses (*p<0.0005). Data are representative of four experiments of five mice per group. PLP₁₃₉₋₁₅₁-specific proliferative responses from Sham Splx and Splx SJL mice were determined on day 10 (FIG. 1J). Data examining the route of inoculation and splenectomized mice were found to be representative of two to three experiments of five mice per group, with scavenger receptor examination determined from one experiment with five mice per group and at least independent spleen sections examined. Asterisks denote a significant reduction of proliferative responses (*p<0.0001).

Splenectomized SJL/J mice responded via DTH (FIG. 1I) and proliferation (FIG. 1J) to PLP₁₃₉₋₁₅₁/CFA immunization similarly to sham-splenectomized control mice; however, splenectomized animals were resistant to tolerance induction with PLP₁₃₉₋₁₅₁-SP, as measured by both assays (FIGS. 1I, 1J). These data show that i.v. administration is likely critical in Ag-SP tolerance induction, most likely due to the direct delivery of apoptotic Ag-SP to immature tolerogenic APCs in the splenic MZ.

### Example 4. Intravenously Infused Ag-SP Locate to the Splenic MZ and are Rapidly Cleared within 18 h

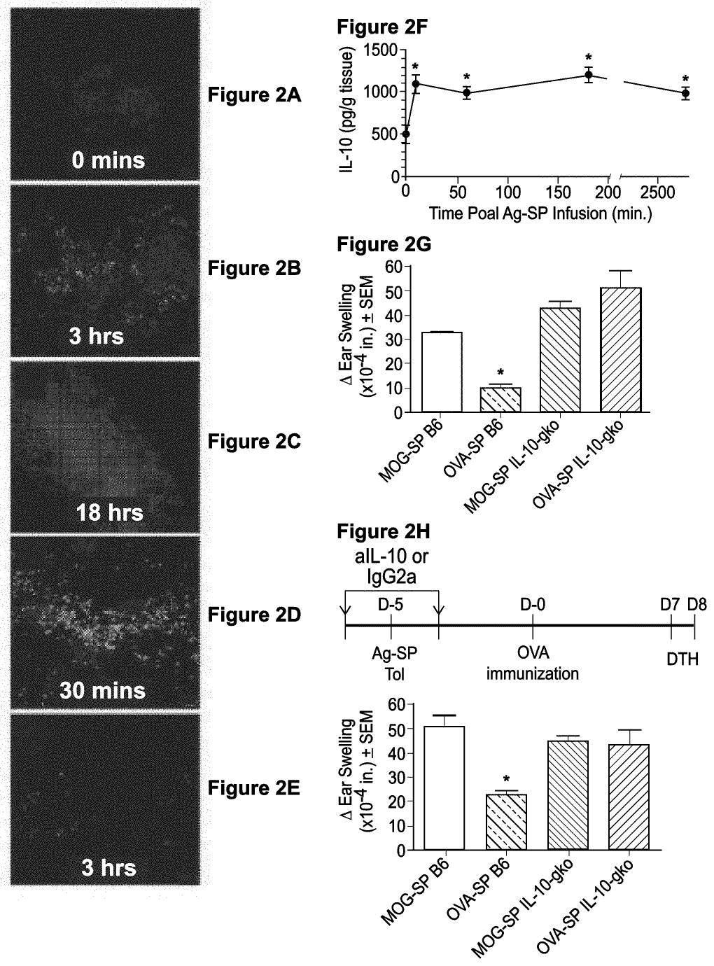

To further elucidate the precise environment of Ag-SP localization within the spleen, the temporal uptake and destruction of PKII-76-labled OVA₃₂₃₋₃₃₉-coupled splenocytes (OVA₃₂₃₋₃₃₉-SP) were examined (FIGS. 2A-C). SJL/J mice were tolerized with 5×10⁷ PKH76-labeled OVA₃₂₃-SP as described in Example 2. Groups of 3-5 mice were sacrificed at 0, 1, 3, and 18 h postinfusion. Within 60 min of infusion, PKH-76-labeled OVA₃₂₃₋₃₃₉-SP were found throughout the spleen, especially within the marginal zone (MZ) (data not shown). At least 20, 8-μM sections were examined from each animal. PKH76-labeled subcellular debris present at 3 h (FIG. 2B) postinfusion was completely absent by 18 h (FIG. 2C). By 3 h postinfusion, the PKH76 staining appeared to be punctate and fragmented, indicating that the Ag-SP had lost cell membrane integrity (FIG. 2B). Using PKH-76 as a marker of membrane debris removal, the Ag-SP were undetectable by 18 h postinfusion (FIG. 2C).

This was further supported by experiments using CFSE labeled Ag-SP (FIGS. 2D, 2E). A separate cohort of at least four animals was treated with 5×10⁷ CFSE-labeled OVA₃₂₃-SP as described in Example 2, and mice were sacrificed 30 min and 3 h postinfusion. Numerous CFSE-labeled Ag-SP were observed at 30 min (FIG. 2D) but were completely absent by 3 h post-infusion (FIG. 2E). At 30 min postinfusion, numerous CFSE-labeled OVA₃₂₃₋₃₃₉-SP can be seen throughout the spleen (FIG. 2D); however, by 3 h postinfusion, no evidence of CFSE-positive cells remains (FIG. 2E). Because CFSE is a cytoplasmic dye, these data suggest that within 3 h, all infused Ag-SP cells lose their plasma membrane integrity, resulting in CFSE diffusion into the extracellular matrix. In contract, PKH-76 is a plasma membrane-bound dye, which will not dilute or leak during the apoptotic process. Therefore, these results collectively indicate that, upon i.v. infusion, the Ag-SP cells rapidly become unstable, lose their membrane integrity within 3 h of infusion, as supported by our earlier finding that ECDI-fixed cells become rapidly apoptotic, and are completely removed by phagocytosis within 18 posttransfer.

### Example 5. IL-10 Secretion is Induced in Response to Intravenous Ag-SP Infusion and is Critical for Tolerance Induction

The rapid clearance of i.v. administered Ag-SP from the spleen (FIGS. 2A-E) suggested that the framework for tolerance induction is initiated very early after Ag-SP infusion. Because of the importance of IL-10 in immune regulation, the level of IL-10 present in whole-spleen homogenates in response to Ag-SP infusion was investigated (FIG. 2F). Groups of at least four mice were infused with 5×10⁷ OVA₃₂₃-SP as described in Example 1. Recipient mouse spleens were harvested at 0, 10, 60, and 180 min postinfusion and IL-10 levels in supernatants of individual homogenized spleens (run in triplicate) were measured using ELISA. IL-10 ELISA was performed using a ready-set-go IL-10 ELISA kit (eBioscience). Spleens from individual mice were snap frozen, defrosted, and homogenized with a handheld homogenizer. The resulting homogenate was ultracentrifuged before IL-10 quantification. IL-10 levels significantly higher than baseline (p<0.01) are marked with * (FIG. 2F). Examination of IL-10 protein revealed that within 10 min postinfusion of OVA₃₂₃₋₃₃₉-SP, IL-10 protein levels increased dramatically. Furthermore, these IL-10 levels remained significantly above the baseline level over the 3 d of testing.

To determine the functional role of IL-10 secretion in Ag-SP tolerance induction, first IL-10-deficient animals (IL-10gko) were tolerized with ECDI-coupled splenocytes coupled with OVA₃₂₃₋₃₃₉-SP. Using DTH as an in vivo measure of T cell tolerance induction in OVA₃₂₃₋₃₃₉/CFA-immunized mice, the results indicated that whereas control mice were successfully tolerized to OVA₃₂₃₋₃₃₉, showing little to no ear inflammation, IL-10gko mice were not tolerized (FIG. 2G). Wild-type (B6) and IL-10-deficient (IL-10gko) C57BL/6 mice were tolerized i.v. with 5×10⁷ syngeneic splenocytes from IL-10gko mice coupled with MOG₃₅₋₅₅ (irrelevant peptide control) or OVA₃₂₃₋₃₃₉ on day −5. On day 0, the mice were immunized with 200 μg OVA₃₂₃₋₃₃₉/CFA, and DTH responses to OVA₃₂₃₋₃₃₁ ear challenge were determined on day 7 (FIG. 2G).

Importantly, donor splenocytes from both wild-type (data not shown) and IL-10gko (FIG. 2G) animals were similarly capable of inducing tolerance in wild-type animals, indicating the source of IL-10 was the recipient. These data are a strong indication of the critical nature of IL-10 for the indication to Ag-SP tolerance. However, IL-10gko mice are known to have altered immune regulation, commonly developing autoimmune conditions, including colitis. Therefore, wild-type B6 mice were administered 100 μg neutralizing IL-10 Ab 30 min prior and 18 h post-OVA₃₂₃₋₃₃₉-SP infusion. IL-10 neutralization was performed through i.p. injection of 100 μg/mouse anti-IL-10 Ab (eBioscience), as described for each experiment. Anti-IL-10 or control IgG2a Ab was given 30 min prior and 18 h after MOG₃₅-SP or OVA₃₂₃-SP infusion of day −5. Animals were immunized with OVA₃₂₃₋₃₃₉/CFA on day 0, and DTH was assessed on day 7. Data in all panels are representative of at least three experiments of at least four mice per group. Asterisks denote a significant reduction in DTH responses (*p<0.0005) as compared with MOG₃₅₋₅₅-SP controls. Whereas immunized mice treated with isotype control Ab displayed a characteristic reduction of DTH responses indicative of tolerance induction, mice receiving anti-IL-10 exhibited ear swelling similar to mice tolerized with the irrelevant MOG₃₃₋₃₅-coupled splenocytes (MOG₃₅₋₅₅-SP) peptide (FIG. 2H). Collectively, these results confirm that the environment supporting Ag-SP tolerance induction is formed early and is critically dependent on IL-10 production.

### Example 6. B Cells are not Required for Ag-SP Tolerance Induction

Previously, it has been reported that the infusion of apoptotic cells with CFA stimulates IL-10-producing regulatory B cells, which can prevent CD4⁺ T cell activation (Gray et al., 2007. Apoptotic cells protect mice from autoimmune inflammation by the induction of regulatory B cells. Proc. Natl. Acad. Sci. USA 104:14080-14085). The importance of both cells in Ag-SP tolerance induction were investigated.

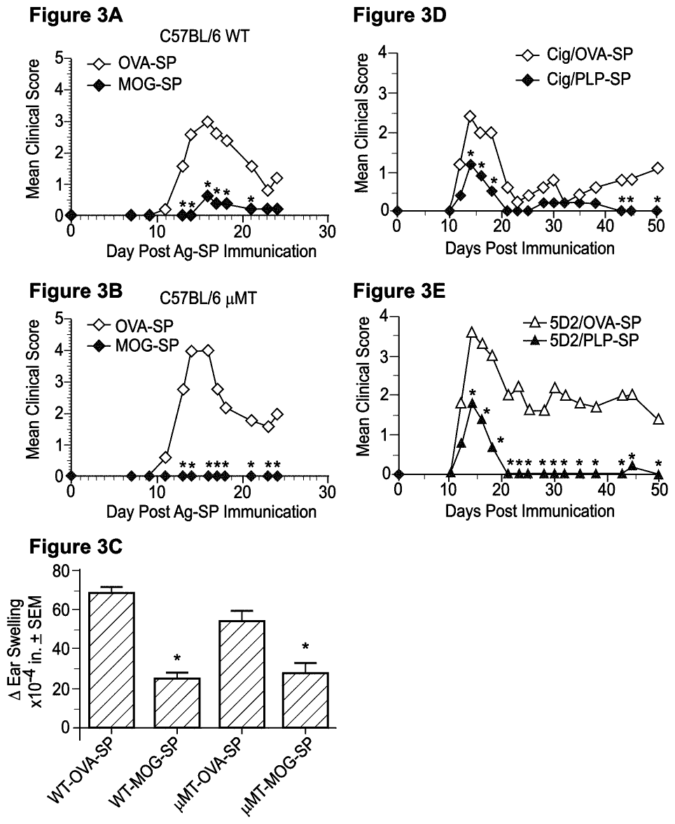

Wild-type (FIG. 3A) and B cell-deficient (pMT) C57BL/6 mice (FIG. 3B) were tolerized i.v. with 5×10⁷ syngeneic MOG₃₅-SP on day −7, primed with MOG₃₅₋₅₅/CFA on day 0, and monitored for clinical EAE disease for 24 d postpriming. Data are representative of two experiments of five mice per group. On day +25 postpriming, MOG₃₅₋₅₅-specific DTH responses were assessed (FIG. 3C). Wild-type SJL/J mice were treated with 250 μg control Ig (CIg: FIG. 3D) or anti-mouse CD20 mAb (FIG. 3E). CD20 depletion was performed using 250 μg/mouse anti-CD20 Ab (clone 5D2 gifted by Genentech) on day −12, followed by i.v. tolerization with 5×10⁷ PLP₁₃₉-SP on day −7. Anti-CD20 treatment resulted in >95% reduction in B cells in the primary lymphoid organs, peritoneal cavity, and the blood within 2 d of Ab injection. On day 0, the mice were primed with PLP₁₃₉₋₁₅₁/CFA and monitored for disease incidence for 50 d postpriming. Data are representative of two experiments of five mice per group. Asterisks denote a significant reduction in mean clinical score or DTH responses (*p<0.01).

In contrast to the Gray et al. study, mice devoid of B cells (pMT mice) can still be tolerized with Ag-SP indicating the B cells are not required for induction of Ag-SP tolerance. Specifically, treatment with MOG₃₅₋₅₅-SP was equally capable of preventing MOG₃₅₋₅₅/CFA-induced EAE in wild-type (FIG. 3A) and pMT mice (FIG. 3B), and tolerance was similarly reflected in MOG₃₅₋₅₅-specific DTH responses (FIG. 3C). In addition, tolerance could be induced in mice depleted of B cells with anti-CD20 (FIG. 3E). These data discount the importance of B cell-derived IL-10 production in Ag-SP tolerance.

### Example 7. Tregs are Critical for Maintenance, but not Induction, of Ag-SP Tolerance

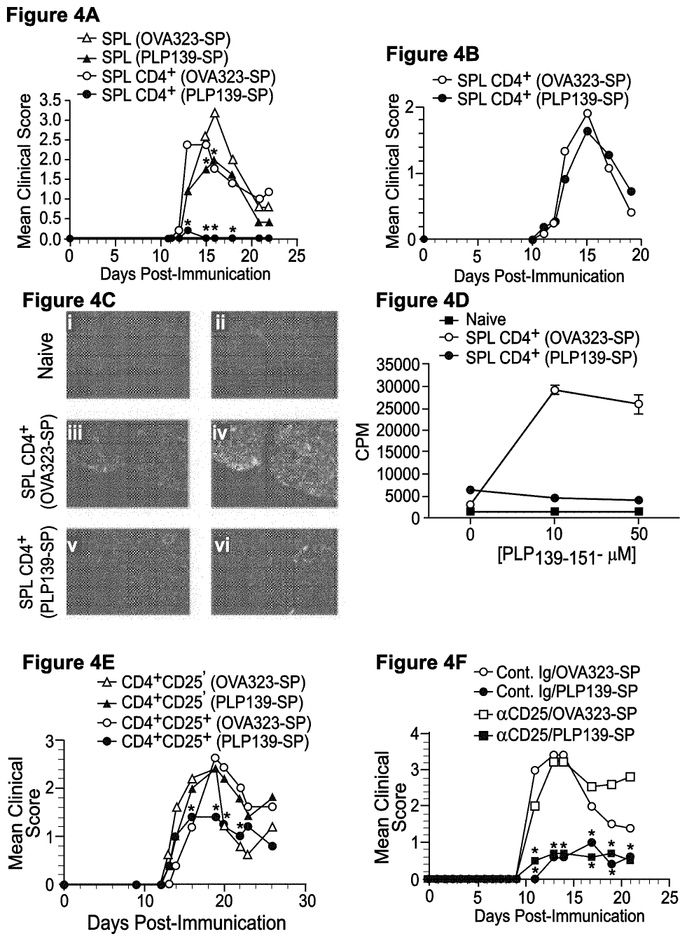

IL-10-producing CD4⁺CD25⁺Foxp3⁺ Tregs have been implicated in immune regulation and tolerance induction in numerous models of inflammation and tolerance. The importance of IL-10 in Ag-SP tolerance suggests that Tregs may also play a role in the induction of Ag-SP tolerance. To address the role to Tregs, mice treated with Ag-SP to test for the ability of transferring tolerance. On day −7, SJL/J mice were tolerized with 5×10⁷ syngeneic splenocytes coupled with either PLP₁₃₉₋₁₅₁ or OVA₃₂₃₋₃₃₉. On day 2, 5×10⁶ bulk splenocytes (SPL) or CD4⁺ splenocytes (SPL CD4⁺) from each treatment group were transferred i.v. to naive recipients that were primed s.c. with 50 μg PLP₁₃₉₋₁₅₁/CFA (FIG. 4A) or PLP₁₇₈₋₁₉₁/CFA (FIG. 4B) on day 0 and monitored for clinical disease. Asterisks denote a significant reduction in clinical score in recipients of bulk or CD4⁺ splenocytes (*p<0.05). Data are representative of two to three experiments of five to eight mice per group. Clinical disease was monitored for 24 d.

Transfer of bulk splenocytes as well as purified CD4⁺ T cells significantly suppressed clinical EAE compared with animals tolerized to the irrelevant OVA₃₂₃₋₃₃₉ peptide (FIG. 4A). Tolerance transfer was Ag specific, as recipients of T cells from PLP₁₃₉₋₁₅₁-SP-treated mice failed to suppress EAE induced by immunization with PLP₁₇₈₋₁₉₁ (FIG. 4B).

Ag-specific regulation was supported by reductions in CNS inflammation, observed by immunofluorescent staining for CD4 T cells and F4/80 (microglia/macrophages) on lumbar spinal cord sections (FIG. 4C i-vi). Two mice from the groups receiving splenic CD4⁺ T cells from OVA₃₂₃-SP and PLP₁₃₉-SP primed with PLP₁₃₉₋₁₅₁/CFA were perfused on day +25. Spinal cords were stained with anti-CD4 (red) or anti-F4/80 (green) mAbs and counterstained with DAPI (blue). Lumbar regions are shown at original magnification ×200.

Ag-specific regulation was also supported by lack of development of PLP₁₃₉₋₁₅₁ proliferative responses (FIG. 4D) in the animals receiving CD4⁺ splenocytes from PLP₁₃₉₋₁₅₁-SP-tolerized animals. Spleens were harvested from three representative mice from each group on day +25, and proliferative responses were determined. Data are representative of two experiments.

These date support an important role for CD4⁺ T cells in disease regulation. To more specifically examine a potential role for Tregs, CD25⁺ and CD25² CD4⁺ splenic T cell populations were purified 5 d post-PLP₁₃₉₋₁₅₁-SP or OVA₃₂₃₋₃₃₉-SP infusion and 5×10⁶ of these cells were transferred independently into naive SJL mice, which were then immunized with PLP₁₃₉₋₁₅₁/CFA and monitored for disease. On day −2, 5×10⁶ CD4⁺CD25⁻ or CD4⁺×CD25⁺ splenocytes from the tolerized mice were transferred i.v. to naive recipients that were primed s.c. with 50 μg PLP₁₃₉₋₁₅₁/CFA and monitored for clinical disease. Asterisks denote a significant reduction in clinical score in recipients of CD4⁺CD25⁺ splenocytes (*p<0.05) from PLP₁₃₉-SP-tolerized mice. Data are representative of two experiments of six to eight mice per group. CD4-CD25, but not CD4⁺CD25⁻ cells, transferred from PLP₁₃₉₋₁₅₁-SP-tolerized animals, induced significant protection from EAE (FIG. 4E).

Overall, these data indicate that CD4⁺CD25⁻ Tregs are a component of tolerance induced by Ag-SP treatment. However, because rapid IL-10 production was observed almost immediately after Ag-SP infusion, and neutralization of IL-10 at the time of Ag-SP was capable of preventing complete tolerance induction (Example 5; FIGS. 2G, 2H), the role of Tregs precisely at the time of tolerance induction was addressed. Using anti-CD25 Ab to deplete/inactivate Tregs, the functional in-activation of Tregs was found to have no measurable effect on tolerance induction, with anti-CD25-treated and isotype control-treated Ag-SP-tolerized animals both exhibiting significantly reduced clinical disease (FIG. 4F). SJL/J mice (5-6 per group) were treated with 500 μg control Ig (Cont. Ig) or anti-CD25 mAb (clone 7D4) on days −11 and −9, tolerized with 5×10⁷ OVA₃₂₃-SP or PLP₁₃₉-SP on day −7, primed with PLP₁₃₉₋₁₅₁/CFA on day 0, and monitored for clinical signs of disease. Data are representative of three separate experiments. Asterisks denote a significant reduction in clinical score of PLP₁₃₉-SP-treated mice (*p<0.01) in both control Ig and anti-CD25-treated mice.

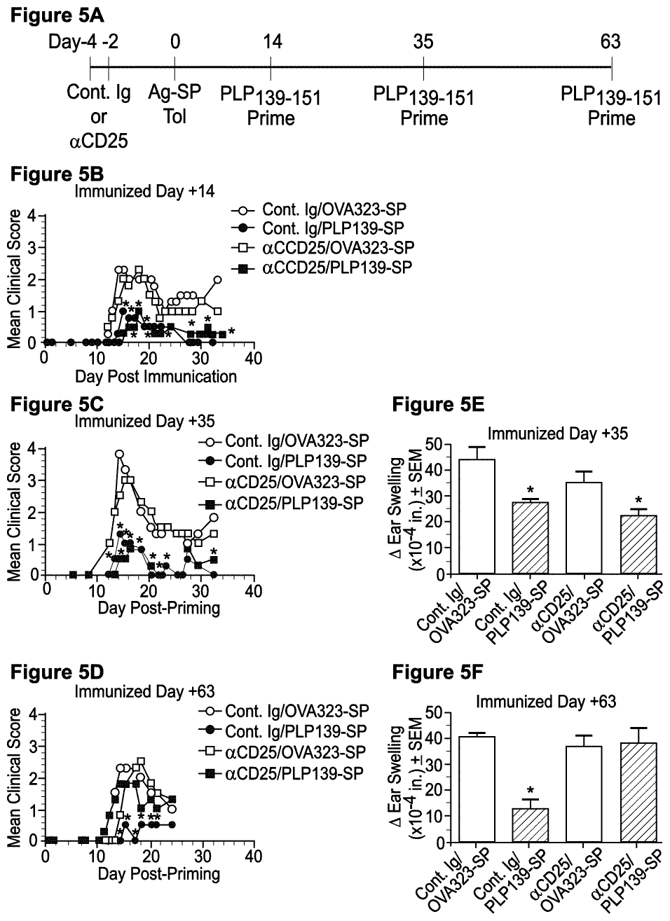

The data suggest that Tregs capable of down-regulating clinical disease are induced by Ag-SP treatment, but that there is a separate nonoverlapping tolerance mechanism induced. According to one hypothesis, whereas Treg may not be critical for tolerance induction, they may play a role in the long-term maintenance of Ag-SP tolerance. To investigate this possibility, a large cohort of SJL/J mice were treated with either control Ig or anti-CD25 Ab (FIG. 5A). SJL/J mice were treated with 500 μg control Ig (Cont. Ig) or anti-CD25 mAb (clone 7D4) on days −4 and −2. On day 0, the entire cohort of mice was tolerized with 5×10⁷ OVA₃₂₃-SP or PLP₁₃₉-SP. Separate groups of mice were primed with 50 μg PLP₁₃₉₋₁₅₁/CFA on day +14 (FIG. 5B), day +35 (FIG. 5C), or day +63 (FIG. 5D) posttolerization and followed for clinical signs of EAE. Data represent the clinical disease pattern of five to six mice per group and are representative of two separate experiments.

DTH responses of mice from FIGS. 5C and D to challenge with PLP₁₃₉₋₁₅₁ were determined following cessation of clinical disease assessment (FIGS. 5E and F). Asterisks denote a significant reduction in clinical disease score (*p<0.01) and DTH responses (p<0.05).

Similarly to data shown above (FIG. 4F), the functional inactivation of Tregs had no effect on tolerance induction in animals immunized on either day 14 (FIG. 5B) or day 35 (FIGS. 5C, 5E), posttolerance induction, as both control Ig and anti-CD25-treated mice tolerized with PLP₁₃₉₋₁₅₁-SP displayed significantly lower clinical disease and peptide-specific DTH. However, only control Ig-treated, not anti-CD25-treated, mice immunized on day 63 post-Ag-SP treatment were protected from disease induction and had significantly downregulated DTH responses (FIGS. 5D, 5F).

Overall, the data support two important observations. First, Tregs do not appear to be required for tolerance induction and are unlikely to be a significant source of the early IL-10 induced by Ag-SP injection. Second, Tregs appear to play a major role in long-term tolerance maintenance for protection from relapsing experimental allergic encephalomyelitis (R-EAE).

### Example 8. Splenic Macrophages Produce IL-10 in Response to Ag-SP In Vivo

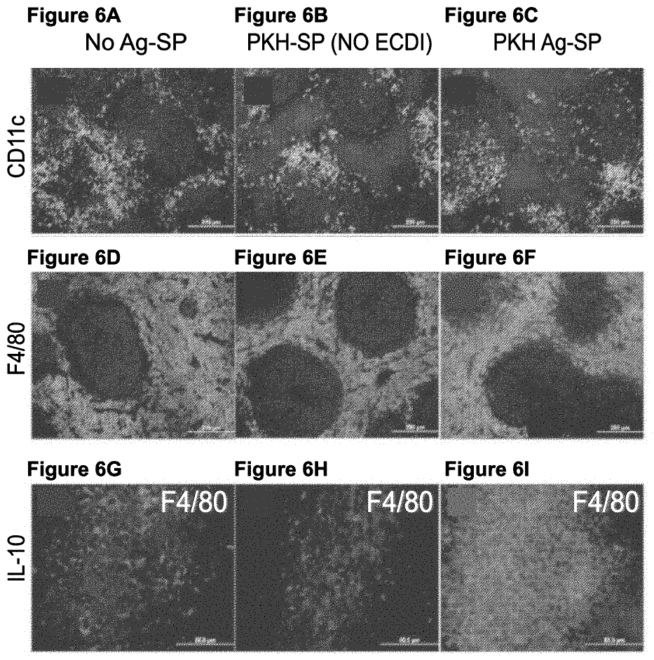

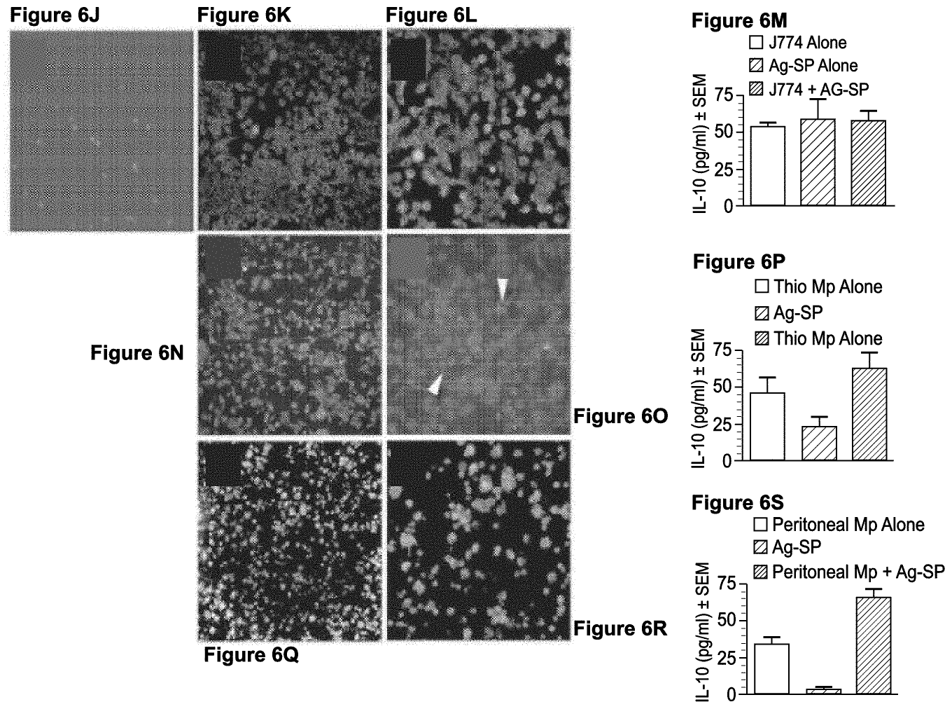

The APC subsets in the spleen involved in tolerance induction were investigated. Using PKH26-labeled Ag-SP (red), the association of Ag-SP with dendritic cells (DCs; CD11c; FIG. 6A-C) or macrophages (F4/80⁺; FIGS. 6D, 6E) at 8 h post-Ag-SP infusion was examined. Groups of at least five C57BL/6 mice were infused with nothing (No Ag-SP, FIGS. 6A, D and G), 5×10⁷ non-ECDI-fixed PKH26 (red)-labeled splenocytes [PKH-SP (No-ECDI), FIGS. 6B, E, and H], or 5×10⁷ ECDI-fixed PKH26-labeled MOG₃₅₋₅₅-SP (PKH Ag-SP, FIGS. 6C, F, and I). Eight hours later, the spleens were harvested for immunohistochemistry. Abs used for immunohistochemistry on spleen sections included rabbit polyclonal hamster anti-mouse CD11c (Biolegend), rat anti-mouse F4/80 (Biolegend), or rat anti-mouse IL-10 (BD Pharmigen). Polyclonal anti-rabbit, hamster IgG, rat IgG2a, or rat IgG1 Abs were used, respectively, as controls (Vector Laboratories, Biolegend, BD Pharmingen). Immunohistochemistry was performed as described in Example 2. Spleen sections (8 μM) were stained in green for CD11c (FIG. 6A-C), F4/80 (FIG. 6D-F), and IL-10 (FIG. 6G-I), and counterstained with DAPI (blue, FIG. 6A-F). Similar to the nonfixed splenocyte control (FIG. 6B), little colocalization of Ag-SP with CD11c was observed (FIG. 6C). F4/80 commonly colocalized with PKH-26 in the Ag-SP-treated animals (FIG. 6F). No IL-10 staining was observed in the untreated (FIG. 6G) or non-ECDI-fixed splenocyte-infused animals (FIG. 6H). Strong IL-10 production (indicated by the green stain) was commonly coincident with F4/80⁺ cells (indicated by the blue stain) (FIG. 6I).

Surprisingly, little PK1126-CD11c colocalization was observed in animals that received either nonfixed splenocytes (FIG. 6B) or ECDI-fixed splenocytes (FIG. 6C). However, fixed splenocytes colocalized at a much high frequency with F4/80-expressing cells (FIG. 6F), especially in the MZ. In addition to the colocalization of Ag-SP with F4/80⁺ macrophages, the expression profile of IL-10 in serial sections in the same experiments was also determined. Although little IL-10 was found in the control nontreated or animals receiving non-ECDI-fixed PKII-26-labeled splenocytes, IL-10 was strongly expressed in Ag-SP recipients. Whereas a small population of cells was found to express IL-10 in the germinal centers of Ag-SP-treated animals (data not shown), the most striking number of cells producing IL-10 were F4/80⁺ and in close proximity to PKH26-expressing Ag-SP (FIG. 6I). Scale bars, 200 μm (FIG. 6A-F), 50 μm (FIG. 6G-I).

### Example 9. Macrophages Produce IL-10 Upon Coculture with Ag-SP

IL-10 responses of a macrophage cell line (J774), as well as primary thioglycolate-stimulated and resting peritoneal macrophages, to coculture with Ag-SP were evaluated. The macrophage cell line, J774 (FIG. 6K-M), thioglycolate-elicited (FIG. 6N-P), and nonelicited peritoneal macrophages (FIG. 6Q-S) were cultured on coverslips in 24-well plates and fed 10⁶ OVA₃₂₃₋₃₃₉-SP labeled with PKH26 (red) overnight. Supernatant was collected for IL-10 analysis, and the remaining coverslips were fixed in paraformaldehyde, counterstained with membrane dye PKH76 (green), and nuclei stained with DAPI (blue). Ag-SP remained PKH26⁺ after overnight incubation; the cells did not label with DAPI or PKH76 (FIG. 6J). J774 macrophages cultured alone (FIG. 6K) and demonstrated uptake of PKH26⁺ cell membranes (FIG. 6L), but failure to produce significant IL-10 (FIG. 6M). Thus, J774 cells phagocytosed PKH-26-labeled (red) Ag-SP, but this uptake failed to upregulate production of IL-10 (FIG. 6K-M).

Thioglycolate-elicited peritoneal macrophages were also found to be capable of ingesting Ag-SP, with a significant amount of PKH-26-labeled membrane localized inside the macrophages, but again, Ag-SP uptake failed to stimulate IL-10 production (FIG. 6N-P). Thioglycolate-elicited peritoneal macrophages cultured alone (FIG. 6N), demonstrated significant uptake of both fragments (white arrowhead) and cells (yellow arrowhead) (FIG. 6O), but failure to produce IL-10 (FIG. 6P). The thioglycolate-stimulated macrophages were rounded up, with multiple nuclei, and exhibited a highly inflammatory phenotype. It was previously shown that lipopolysaccharide (LPS) injection is capable of preventing Ag-SP tolerance in vivo. Because the J774 macrophage line and the thioglycolate-elicited peritoneal macrophages are of a type 1 phenotype, characterized by the production of proinflammatory cytokines, one reasonable hypothesis is that the normal response to Ag-SP is overcome by the background activation state of these cells. Thus, the nonelicited peritoneal macrophages harvested from multiple mice were tested. These cells were found to exhibit the greatest capacity to ingest Ag-SP as determined by the internalization of PKH26-labeled membrane material, and, importantly, that resting macrophages produced significantly upregulated levels of IL-10 upon ingestion of OVA₃₂₃₋₃₃₉-SP (FIG. 6Q-S). Resting peritoneal macrophages were cultured alone (FIG. 6Q) and demonstrated significant uptake of PKH26-labeled Ag-SP (FIG. 6R) and significant production of IL-10 (FIG. 6S). Data represent at least six independent wells, conducted in two or three separate experiments. Asterisk represents significant increase in the level of IL-10 (p<0.05).

The production of IL-10 by these macrophages is consistent with our in vivo immunohistological findings (FIG. 6I) as well as observations by other investigators examining the response of macrophages to tolerogenic stimuli. In conclusion, these results support a scenario in which resting MZ macrophages respond rapidly to Ag-SP and are likely to be the major source of the early IL-10 produced in response to i.v. Ag-SP infusion and to be critical for the induction of tolerance.

### Example 10. Splenic Macrophages Uptake Ag-SP and Express PD-L1 in an IL-10-Dependent Manner

The data generated in the previous examples indicate that the long-term Ag-specific tolerance triggered by the infusion of Ag-SP is the result of more than one mechanism, with Tregs primarily required for tolerance maintenance. Next, it was investigated whether there was a role for CD8a⁺DCs, as well as different macrophage subpopulations, in Ag-SP uptake, as a source for IL-10 and/or as major drivers of Ag-SP tolerance.

Effect of Ag-SP infusion on splenic macrophage ratio was investigated. Five groups of SJL/J mice (four to five mice per group) received IgG2a control Ab, anti-IL-10 alone, OVA₃₂₃₋₃₃₉-SP+IgG2a Ab, OVA₃₂₃₋₃₃₉-SP+anti-IL-10 Ab, or no treatment. All Abs were given 30 min prior to OVA₃₂₃-SP infusion. Three hours after infusion, animals were sacrificed and splenocytes stained with a mixture of Abs. Cells were isolated from the spleen, as described in Bailey et al., (2007, CNS myeloid DCs presenting endogenous myelin peptides ‘preferentially’ polarize CD4+T (H)-17 cells in relapsing EAE. Nat. Immunol. 8: 172-180.)

FcR blocking with CD16/32 was performed. Cells were then stained with either a mixture of Abs containing CD4 (BD Biosciences), CD11c (BD Biosciences), CD8 (BD Biosciences), B220 (BD Biosciences), F4/80 (Biolegend), and PD-L1 (BioXcell), or respective isotype controls. Samples were run on a FACSCanto flow cytometer with FACS DIVA software (BD Biosciences). PD-L1 expression was determined based on mean fluorescent intensity relative to isotype controls.

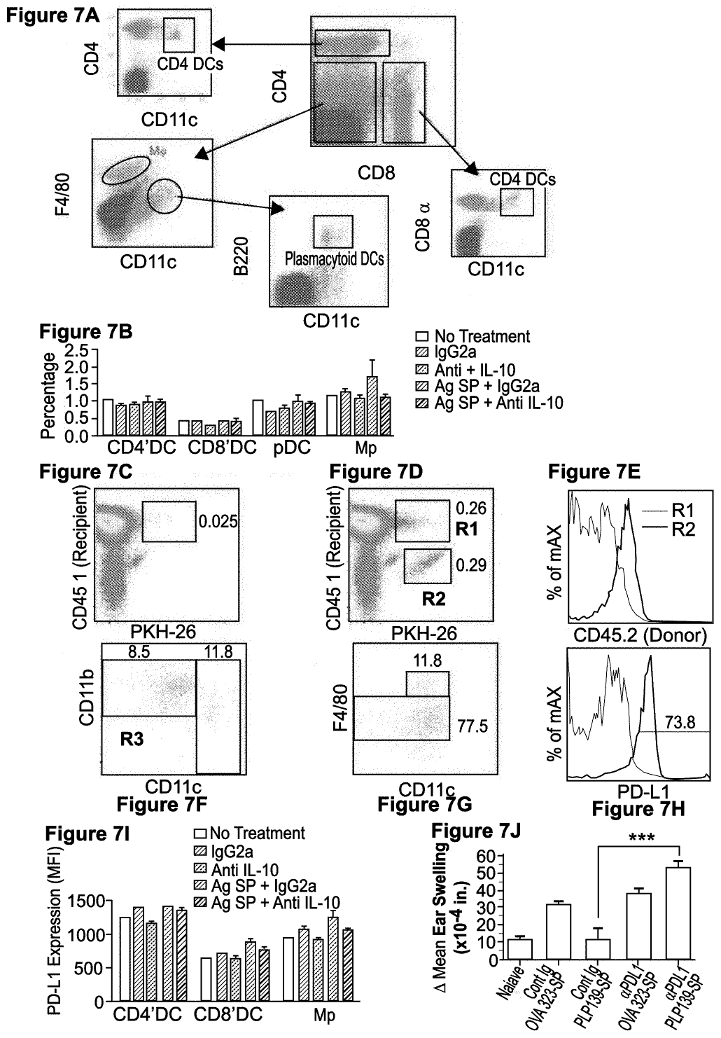

Splenic APC populations were enumerated using the gating strategy shown; black population indicates the ungated isotype control for each dot plot (FIG. 7A). Percentages of CD4⁺, DCs, CD8α⁺ DCs, and plasmacytoid DCs did not change in any of the treatment groups, but percentages of macrophages increased in an IL-10-dependent fashion (FIG. 7B).

Examination of the ratio of APC populations in the spleen 3 h after Ag-SP infusion revealed that the ratio of the major DC subsets, including CD4⁺ DCs (CD4⁺CD11c⁺CD8a²), CD8a⁺DCs (CD8a⁺CD11c⁺CD4²), and plasmacytoid DCs (CD8²CD4² CD11c^{int}), remains unchanged, and was further unaffected in mice treated with anti-IL-10 (FIG. 7A, 7B). In contrast, F4/80− expressing macrophages (F4/80⁺CD11C²CD4²CD8²) significantly increased in relative percentage within 3 h post-Ag-SP infusion (FIG. 7B).

To further examine the potential uptake of Ag-SP be macrophages, OVA₃₂₃-coupled B6 CD45.1 congenic splenocytes were labeled with PKH-26 and injected into CD45.1 mice, which were sacrificed 3 h after infusion. Spleens from CD45.1⁺ C57BL/6 mice receiving either PBS (FIG. 7C) or 5×10⁷ CD45.2⁺ PKH-26-labeled OVA₃₂₃-SP (FIG. 7D) were harvested 2.5 h after i.v. administration. Gate R1 represents recipient cells that have taken up donor Ag-SP, whereas gate R2 represents intact Ag-SP. Numbers adjacent to gate represent the percentage of cells within the gate (FIG. 7D).

Whereas no PKH-26 colocalization was observed with recipient CD45.1 in untreated controls (FIG. 7C), 3 h after Ag-SP infusion there was a distinct population of PKII-26-/CD45.1⁺ cells as well as possibly intact PKH26⁺ CD45.1² donor cells (gate R1; FIGS. 7D, 7E).

Relative CD45.2 expression on gates R1 (gray line) and R2 (black line) are displayed on FIG. 7E. Cells from gate R1 were 85% CD11b and 11.6% CD11c^{high} (FIG. 7F) The PKH-26⁺CD45.1 cells were 85% CD11b⁺F4/80¹/CD11c^{2/low} (gate R3; FIG. 7F), with only 11.6% expressing CD11c^{high} (FIG. 7F). Cells from gate R3 were 77.5% F4/80^{int} and 11.3% F4/80^{high} (FIG. 7G). The majority of the cells in gate R3 were CD11c^{int}, which is consistent with the phenotype of splenic MZ macrophages (FIG. 7G). Significantly, PKH-26-colocalized macrophages also expressed high levels of PD-L1 (FIG. 7H). Greater than 73% of cells from R3 (i.e., those that are of recipient origin, the majority being F4/80⁺ macrophages) that have engulfed Ag-SP expressed PD-L1 (FIG. 7H). These results show that shortly after i.v. infusion of Ag-SP, macrophages not only change in their relative percentage in the spleen, but they are also the major population taking up the apoptotic Ag-SP debris and expressing PD-L1.

### Example 11. PD-L1 Expression was Required for Ag-SP Tolerance

Interestingly, administration of IL-10-neutralizing Ab 30 min prior to Ag-SP infusion completely abrogated the increase in F4/80-expressing macrophages (FIG. 7B), suggesting that IL-10 may play a role in the overall kinetics of cellular proliferation/migration within the splenic microenvironment. PDI/PD-L1 and IL-10 have been reported to reciprocally regulate each other. Therefore, PD-L1 expression on APC populations after Ag-SP infusion was examined.

PD-L1 expression increased in the CD8α⁺ DC and F4/80⁺ macrophage populations, and expression was reversed by anti-IL-10 in macrophages (FIG. 7I). Data are representative of two separate experiments. Asterisks denote a significant change in APC subset ratio/expression compared with animals treated with IgG2a control AB (*p<0.05).

Within 3 h, macrophages displayed the greatest increase in PD-L1 expression (FIG. 7I). This was not reflected by increase expression of other molecules such as MHC-II and CD80/86, which remained unchanged on all examined APC populations (data not shown). Interestingly, CD8a⁺CD11c⁺CD4² DCs also upregulated PD-L1 within the time frame examined; however, unlike the macrophages, neutralization of IL-10 did not significantly alleviate PD-L1 expression on DCs.

### Example 12. PD-L1 Blockade Prevented AG-SP Tolerance Induction

Finally, to address the functional role of PD-L1 in tolerance induction, anti-PD-L1 Ab was infused at the time of PLP₁₃₁₋₁₅₁-SP infusion and the mice were subsequently primed with PLP₁₃₉₋₁₅₁/CFA. SJL/J mice were treated with anti-PD-L1 or control IgG2a Ab. Mice were treated i.p. with 500 μg anti-PD-L1 (clone 10F.9G2) or with control rat IgG2b on day −7, and additionally with 250 μg on days −5, −3, −1, and +1 relative to immunization with PLP₁₃₉₋₁₅₁/CFA. Anti-PD-L1 and isotype control rat IgG2b Abs were purchased from BioXcell Fermentation and Purification Services (West Lebanon, N.H.). DTH was accessed on day 7. Results are representative of two separate experiments of at least five mice per group. Asterisks denote a significant reduction in DTH responses (*p<0.01) as compared with MOG₃₅₋₅₅-SP controls.

Animals became moribund within 14 d postimmunization (data not shown). It has been described previously that administration of anti-PD-L1 Ab early in EAE induction can significantly exacerbate disease. Because of the severe disease phenotype observed, we examined tolerance induction in anti-PD-L1-treated animals by DTH at 7 d postpriming. PD-L1 inhibition at the time of Ag-SP infusion completely ablated the induction of tolerance (FIG. 7J). Based on the disease observations, Ag-SP infusion in the presence of anti-PD-L1 may prime mice such that later immunization with PLP₁₃₉₋₁₅₁/CFA results in lethal EAE. Collectively, the data suggest a critical role for IL-10 and build on our and others previous findings regarding the importance of PD-L1 in the induction of Ag-SP tolerance. IL-10 regulates PD-L1 expression of F4/80 macrophages, which appears important for Ag-SP tolerance induction.

### Example 13. Preparation of Primary Reagents for Inducing Tolerance in Humans Using Autologous Blood Cells

To prepare the blood cells, the excipients crythrocyte lysis buffer and peptide solution are produced in advance and stored at <−20° C. The peptide solution is prepared in the clean room (Category A). First, 30 (±3) mg of each single peptide are weighed in and solved in 7.5 ml of water for injections (final concentration of peptide 4 mg/ml), respectively. Thereafter, all peptides are pooled by transferring 5 ml of each single-peptide solution into a new tube and adding 5 ml of water for injections (total volume 40 ml) to obtain a final concentration of 0.5 mg/ml of each single peptide. Peptide-pool solution is aliquoted in 1.5 ml aliquots (20 aliquots) in sterile and endotoxin free NUNC Cryo Tube vials (NalgeNunc International) and stored at −200 C until use. 5 ml of the Peptide-pool solution are transferred into a blood-bag containing 30 ml of water for injections for sterility testing. 5 ml are aliquoted at 1 ml and stored at −20° C. for later quality controls. Peptide-pool solutions are passed through sterility control before they are used in the manufacture process. The identity and presence of each single peptide in the pool is verified.

At the day of manufacture of drug product, 1 ml of peptide-solution is transferred to a blood bag (P1459, Fresenius; see IMPD 2.1.P.3.5 Filling of blood bags in clean room). The procedure is done in the clean room (category A). The blood bag containing the peptide solution is stored at 4° C. until use.

The preparation of the erythrocyte lysis buffer is done in the clean room. g of Ammonium chloride EMPROVE® Ph Eur and g of Potassium hydrogen carbonate EMPROVE® Ph Eur are solved in 50 ml of water for injection (Ph Eur). Using a 50 ml syringe, 25 ml of the solved lysing buffer are transferred to a blood bag through a sterile filter (0.2 μm, Millipore). The blood bag is filled up to 200 ml with water for injection and stored at −20° until use. Two bags are filled. 50 ml of lysis buffer are transferred to a blood bag for sterility testing and 50 ml are preserved at −20° C. for later quality control. Erythrocyte lysis buffer solutions are passed through sterility control before they are used in the manufacture process. To prepare the CPD/saline washing solution, a CPD bag (Compoflex, Fresenius) containing 63 ml of CPD is filled up to 500 ml with sterile physiologic saline (NaCl 0.9%, Baxter) solution. Bags are connected by TSCD. A balance (PC4000, Mettler) is used to control for weight (50 Og). Two bags are produced. At the end of the manufacture process residual washing solution is tested for sterility. To prepare the EDC solution, 200 mg EDC are solved in 2 ml of water for injection in the clean room (Cat. A). Using a sterile syringe, 1 ml is transferred to a blood bag (P1459, Fresenius). The blood bag with the EDC solution is stored at 4° C. until use. Residual EDC is tested for sterility. At the day of blood collection, 2.5-5×10⁹ PBMC is isolated from qualifying MS patients by standard leukapheresis. For the collection of cells, a standardization automatic program (AutoPBSC) on a Cobe Spectra apheresis machine (Cone Spectra) is used. The AutoPBSC processes 4500 ml of blood and enriches PBMC in 6 harvest phases with approximately 10 ml volume each. In parallel to the collection of cells, 120 ml of autologous plasma is collected during the apheresis procedure and stored at 4° C. in a standard blood bag. During the whole apheresis procedure ACD-A (Baxter) is used as anticoagulant to prevent clotting of blood. The AutoPBSC program uses ACD-A at 0.083 ml/ml (relation 1:12), however the amount is adapted within defined ranges (0.071-0.1 ml/ml), as necessary. At the end of the apheresis the concentration of ACD-A in the cell product and plasma is recorded.

All steps described herein for cell processing are done maintaining a closed system. Excipients are pre-filled in blood bags in the clean room (category A) and added to the cells by connecting the bags using a sterile tubing welder (TSCD®, Terumo). The apheresate is transferred to a standard blood bag (Compoflex P1461 500 ml, Fresenius) by welding the tubes of the bags with the TSCD®. A small retention sample is maintained in the original blood bag that will be used for counting of cells after bags have been separated using a portable tubing sealer (Fresenius NBPI). Cells are separated from plasma by centrifugation at 300×g for 15 min at room temperature (RT). Plasma is removed from the bag by pressing it to a sterile connected empty bag, using a plasma extractor (Baxter). The bags are separated by a portable tubing sealer. To lyse erythrocytes the bag containing the erythrocyte lysing buffer (ACK) is connected by the TSCD and the cell pellet is resuspended in 200 ml erythrocyte lysis buffer and incubated for 15 min, RT, shaking (3 rpm) on a wave platform shaker (Heidolph). At the end of the incubation period cells are washed with 200 ml CPD 12.6%/saline and centrifuged for 15 min at 200 g at 4° C. Supernatant is removed from the bag by pressing it to a empty bag, using a plasma extractor. The cells are washed again with 200 ml CPD 12.6%/saline. Cells are centrifuged for 15 min at 200 g at 4° C. and supernatant is removed from the bag. Cells are transferred to a 150 ml bag (Compoflex 1459, Fresenius) and a retention sample is taken for cell counting.

### Example 14. Preparation of Peptide Labeled Blood Cells Generated by ECDI Coupling for Tolerance Induction in Humans

The use of medicinal product for human are (ETIMS) containing autologous peripheral blood mononuclear cells (PBMC) cells that have been pulsed with six, seven or eight of eight immunodominant myelin peptides (MBP 13-32: KYLATASTMDHARHGFLPRH (SEQ ID NO: 1), MBP 83-99: ENPWHFFKNIVTPRTP (SEQ ID NO: 2), MBP 111-129; LSRFSWGAEGQRPGFGYGG (SEQ ID NO: 3), MBP 146-170: AQGTLSKIFKLGGRDSRSGSPMARR (SEQ ID NO: 4), PLP 139-154: HCLGKWLGHPDKFVGI (SEQ ID NO: 5), MOG 1-20: GQFRVIGPRHPIRALVGDEV (SEQ ID NO: 6), MOG 35-55: MEVGWYRPPFSRWHLYRNGK (SEQ ID NO: 7), MBP 82-98 DENPWHFFKNIVTPRT (SEQ ID NO: 8) and fixed with the cross-linker ECDI is tested.

In addition, four cocktails containing autologous blood cells that have been pulsed with a cocktail of peptides were prepared as follows:

a) MBP 13-32, MBP 83-99, MBP 111-129, MBP 146-170, PLP 139-154, MOG 1-20 and MOG 35-55

b) MBP 13-32, MBP 82-98, MBP 111-129, MBP 146-170, PLP 139-154, MOG 1-20 and MOG 35-55

c) MBP 13-32, MBP 83-99, MBP 111-129, MBP 146-170, PLP 139-154, MOG 1-20, MOG 35-55 and MBP 82-98

d) MBP 13-32, MBP 111-129, MBP 146-170, PLP 139-154, MOG 1-20 and MOG 35-55.

The cells are fixed with the cross-linker ECDI.

1.5-2×10⁹ PBMC is re-suspended in 10 ml saline and 1 ml peptide-pool solution containing 0.5 mg/ml of each GMP manufactured peptide added. The selected peptides (e.g., MBP1, MBP2, MBP3, MBP4, PLP1, MOG1 and MOG2) are used for coupling. The coupling reaction is initiated by the addition of 1 ml of 100 mg/ml of freshly prepared water-soluble 1-ethyl-3-(3-dimethylaminopropyl-)-carbodiimide (EDC). Following 1 h incubation shaking at 4° C., the peptide-coupled cells are washed 2 times with 100 ml CPD/saline and finally re-suspended in autologous plasma at a concentration given by the specification (1×10⁵, 1×10⁶ or 1×10⁷ cells/ml). At this time sample is taken for release testing prior to infusion. Cells are carefully checked for the absence of clumping. 100 ml of final ETIMS cell product is infused using a standard blood transfusion kit with inline-filter (200 μm). The control of critical steps and intermediates are well known in the art and described in patent publication WO 2009/056332, which is incorporated by reference in its entirety.

The whole manufacture process is performed within standard blood bags in a functionally closed system. Peptides, lysis buffer and washing solutions are filled in standard blood bags under sterile and endotoxin free conditions in a licensed clean room laboratory (category A, ISO14644 certified) following strict GMP standards. In the manufacture process the addition of these materials/reagents are carried out by welding the tubes of the respective blood bags with a sterile tubing welder (Terumo TSCD®).

An initial cell number of higher than 1.2×10⁸ is used. Cell counts are assessed before starting the manufacture process, before the coupling reaction and after the last washing step to reach a target cell count of 5×10⁸. The pH of the product after resuspension is checked for a target of ˜7.7. The cell viability is assessed by measuring membrane integrity by Trypan blue exclusion and FACS (Ph. Eur. 2.7.29) at different time-points and storage conditions. To assess the efficiency of peptide coupling reaction to the surface of the PBMC, one of the seven peptides (PLP139-154) has been replaced by a biotinylated peptide (biotinPLP139-154). Binding of the peptide to the surface of the cells has been detected by FACS and fluorescence microscopy using fluorophore conjugated streptavidin (Streptavidin-Cy3 and Streptavidin-APC respectively). Suitable ranges of reagent concentrations and reaction volumes are described in patent publication WO 2009/056332, which is herein incorporated by reference in its entirety. The products are checked for sterility, endotoxins and aggregates. Suitable methods to assess sterility, endotoxins and aggregates are described in patent publication WO 2009/056332, which is incorporated herein by reference in its entirety.

### Example 15. Administration of Peptide Labeled Blood Cells Generated by ECDI Coupling for Tolerance Induction in Humans

1.5-2×10⁹ peripheral blood mononuclear cells are isolated from a MS patient. The isolated cells are coupled according to the manufacture process described in Example 14. Four cocktails are prepared as described in Example 14. The resulting suspensions of approximately 10⁹ cells suspended in 100 ml water buffered to pH 7.2-7.8 is infused intravenously to the patient. MRI examinations are carried out before and after application (e.g., 1 day, 1 week, 1 month, 6 months, 1 year after application) to demonstrate the efficacy of the procedure in terms of reduction of CNS-inflammation. Patients are observed for clinical symptoms associated with MS that are known in the art.

### Example 16. Administration of Peptide Coupled PLG Microspheres for Tolerance Induction to MS-Antigen in Humans

Fluoresbrite® YG Carboxylate microspheres are purchased from Polysciences, Inc. (Warrington, Pa.). The peptides are coupled to the microspheres using ECDI to a specific number of active amino or carboxyl sites on the particles. Microspheres are washed 2× in PBS, resuspended at 3.2×10⁶/ml in PBS with 1 mg/ml of each peptide and 30.75 mg/ml ECDI (CalBiochem, La Jolla, Calif.), and incubated for 1 h at 4° C. with periodic shaking. Peptide-coupled microspheres are then washed 3× in PBS, filtered through a 70 μm cell strainer, and resuspended at 250×10⁶ microspheres/ml in PBS. The microspheres are coupled with the eight antigenic peptides described in Example 14. Cocktails of microspheres coupled with combinations of two or more peptides are prepared. In separate batches, microspheres are also coupled with a single antigenic peptide each. Cocktails of the singly coupled microspheres are also prepared in different combinations.

The resulting cocktails are administered i.v. to MS patients. MRI examinations are carried out before and after application (e.g., 1 day, 1 week, 1 month, 6 months, 1 year after application) to demonstrating the efficacy of the procedure in terms of reduction of CNS-inflammation. Patients are observed for clinical symptoms associated with MS that are known in the art. Clinical symptoms and indicators of CNS inflammation are reduced after administration of the cocktails.

### Example 17. Administration of Peptide Coupled PLG Microspheres Encapsulating Regulatory Cytokines for Tolerance Induction to MS-Antigens in Humans

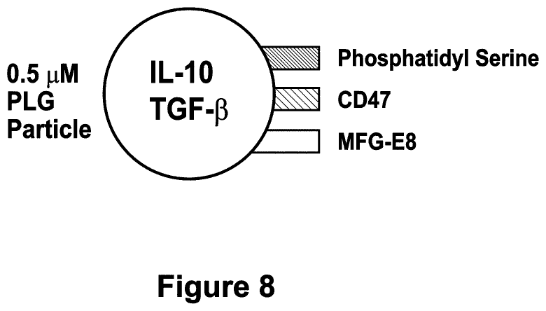

Peptide coupled 0.5 μm poly(lactide-co-glycolide) (PLG) microspheres and cocktails thereof are prepared as described in Example 16 further encapsulating IL-10, TGF-β or both (FIG. 8). The resulting cocktails are administered i.v. to an MS patient. MRI examinations are carried out before and after application (e.g., 1 day, 1 week, 1 month, 6 months, 1 year after application) to demonstrate the efficacy of the procedure in terms of reduction of CNS-inflammation. Patients are observed for clinical symptoms associated with MS that are known in the art.

### Example 18. Administration of Peptide Coupled PLG Microspheres Encapsulating Regulatory Cytokines and Flagged with Apoptotic Tags for Tolerance Induction to MS-Antigens in Humans

Peptide coupled 0.5 μm PLG microspheres and cocktails thereof are coupled with phosphatidyl serine and prepared as described in Example 16 further encapsulating IL-10, TGF-3 or both. The resulting cocktails are administered i.v. to an MS patient. MRI examinations are carried out before and after application (e.g., 1 day, 1 week, 1 month, 6 months, 1 year after application) to demonstrate the efficacy of the procedure in terms of reduction of CNS-inflammation. Patients are observed for clinical symptoms associated with MS that are known in the art.

### Example 19. Antigen Specific Personalized Therapy Comprising Administration of Antigen Coupled PLG Microspheres

A patient in tested for an immune reaction and the identity of one or more immunogenic antigens are determined using suitable methods known in the art. Microspheres are prepared as described in Examples 16, 17 or 18 using the identified immunogenic antigens in place of the listed peptides in the examples. The preparation is administered i.v. to the patient. The patient is observed for immune response to the one or more antigens before or after the administration (e.g., 1 day, 1 week, 1 month, 6 months, 1 year after application) to demonstrate the efficacy of the procedure in terms of inducing tolerance using suitable methods known in the art.

### Example 20. Production and Use of Peptide-Coupled Polystyrene Microspheres. Production of Peptide-Coupled Polystyrene Microspheres

If necessary, the carboxyl microparticles, PolyLink Coupling Buffer and PolyLink Wash/Storage Buffer (Polysciences, Inc. Warrington, Pa.) are warmed to room temperature. Carboxyl (COOH) microparticles can be used for covalent coupling of proteins by activating the carboxyl groups with water-soluble carbodiimide (ECDI). The carbodiimide reacts with the carboxyl group to create an active ester that is reactive toward primary amines on the protein of interest. 12.5 mg of microparticles are pipetted into a 1.5 polypropylene microcentrifuge tube and pelleted via centrifugation for 5-10 minutes at approximately 10000×G. The microparticle pellet is resuspended in 0.4 ml of PolyLink Coupling Buffer and pelleted again via centrifugation for 5-10 minutes at approximately 10000×G. The microparticle pellet is resuspended in 0 17 ml of PolyLink Coupling Buffer. Just before use, a 200 mg/ml ECDI solution is prepared by dissolving 10 mg PolyLink ECDI in 50 μl PolyLink Coupling Buffer. 20 μl of the ECDI solution is added to the microparticle suspension and mixed gently end-over-end or briefly vortexed. Peptides (e.g., PLP₁₃₉₋₁₅₁ or OVA₃₂₃₋₃₃₉) equivalent to 200-500 μg is added and mixed gently by pipetting. The mixture is incubated for 30-60 minutes at room temperature and then centrifuged for 10 minutes at approximately 10000×G. This supernatant is saved for determination of the amount of bound protein. The microparticle pellet is resuspended in 1 mL sterile PBS and centrifuged again in 10000×G.

### Example 21. Peptide-Coupled Polystyrene Microspheres Induce Specific Tolerance for Both Prevention and Treatment of PLP-Induced EAE

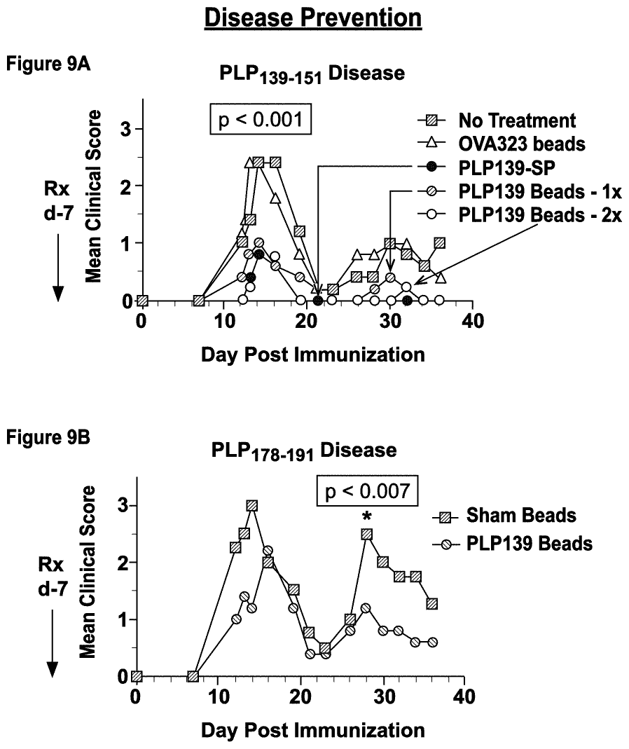

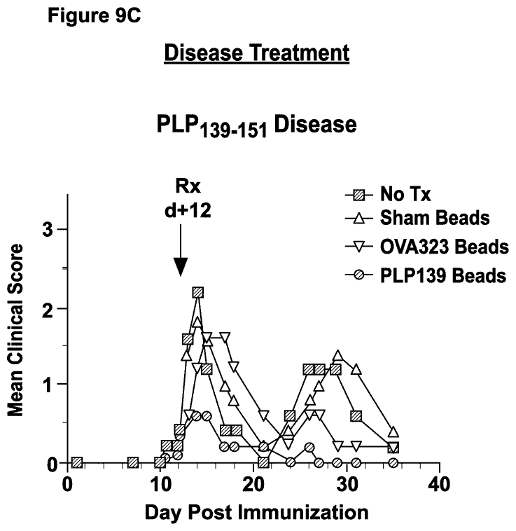

The effect of administration of peptide-coupled polystyrene microspheres is tested either prior to, or after induction of PLP₁₃₉₋₁₅₁ induced EAE in mice. The production of peptide coupled microspheres was performed either as described in Example 16 or Example 20. Either PLP₁₃₉₋₁₅₁ or a control (OVA₃₂₃₋₃₃₉) peptide was coupled to 0.5 μm microspheres. Mice were injected intravenously with either the PLP₁₃₉₋₁₅₁ or control (OVA₃₂₃₋₃₃₉) peptide bound microspheres either on day −7 (“Disease Prevention”) or day 12 (“Disease Treatment”) relative to priming with PLP₁₃₉₋₁₅₁ or PLP₁₇₈₋₁₉₁+Complete Freund's Adjuvant (CFA) on day 0. Individual animals were observed every 1-3 days, and clinical scores were assessed on a scale of 0-4 as follows: 0=no abnormality; 1=limp tail or hind limb weakness; 2=limp tail and hind limb weakness; 3=partial hind limb paralysis; 4=complete hind limb paralysis. Data are reported as the mean daily clinical score. Mice were observed for clinical signs of EAE for an additional 40 days. The results are shown in FIG. 9. Animals treated prior to disease onset with PLP₁₃₉₋₁₅₁-coated microspheres showed a decrease in clinical score compared to animals treated with Sham beads (microspheres treated with ECDI but no peptide) (FIG. 9C). The results also showed a similar decrease in clinical score to treatment using cells treated to have PLP₁₃₉₋₁₅₁ on the cell surface (PLP₁₃₉₋₁₅₁-SP; see FIGS. 9A and 9B). Animals treated following disease onset with PLP₁₃₉₋₁₅₁-coated microspheres similarly showed a decrease in clinical score compared to animals that were either untreated or treated with microspheres having a control peptide (see FIG. 9C). Therefore, the results show that treatment using peptide-coupled polystyrene microspheres is useful for decreasing disease severity prior to and following disease onset.

### Example 22. Preferred Route and Size for Tolerance Induction Using Peptide-Coupled Polystyrene Microbeads

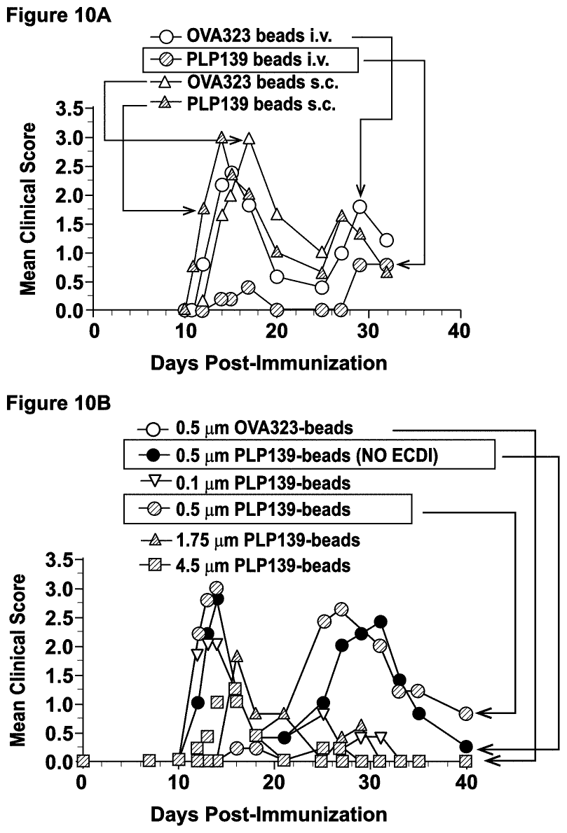

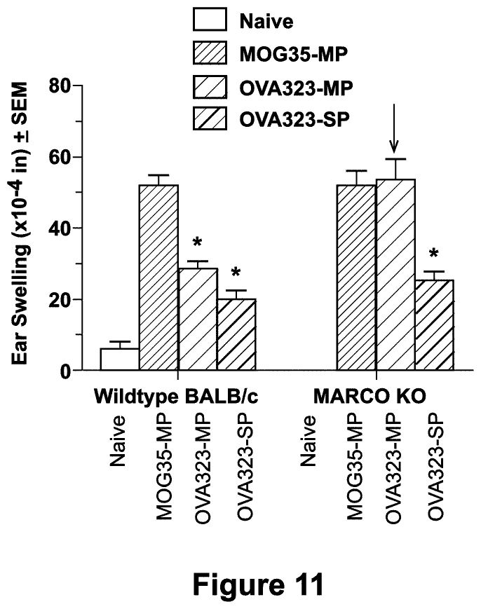

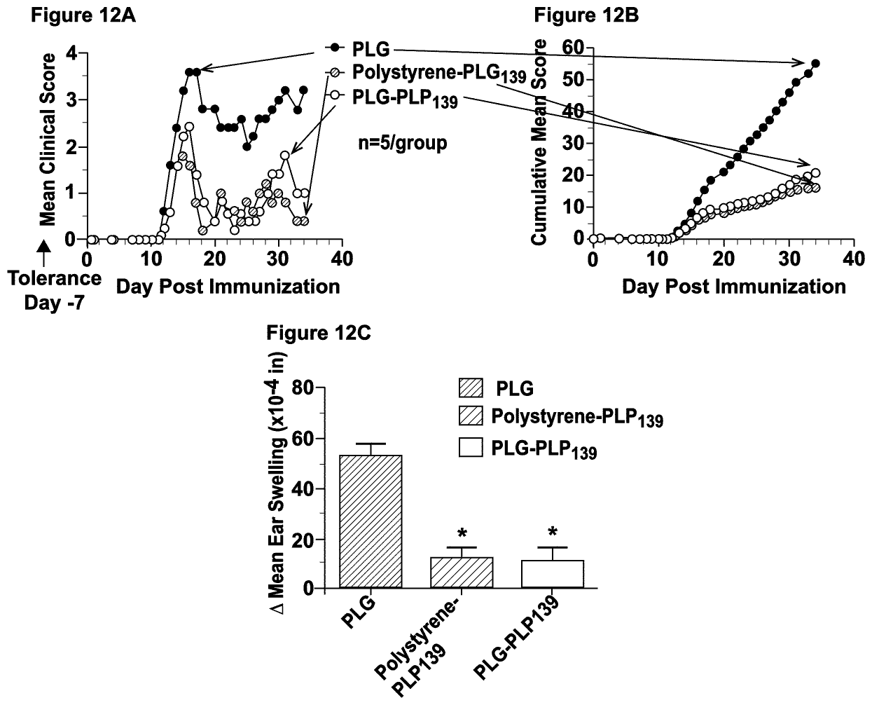

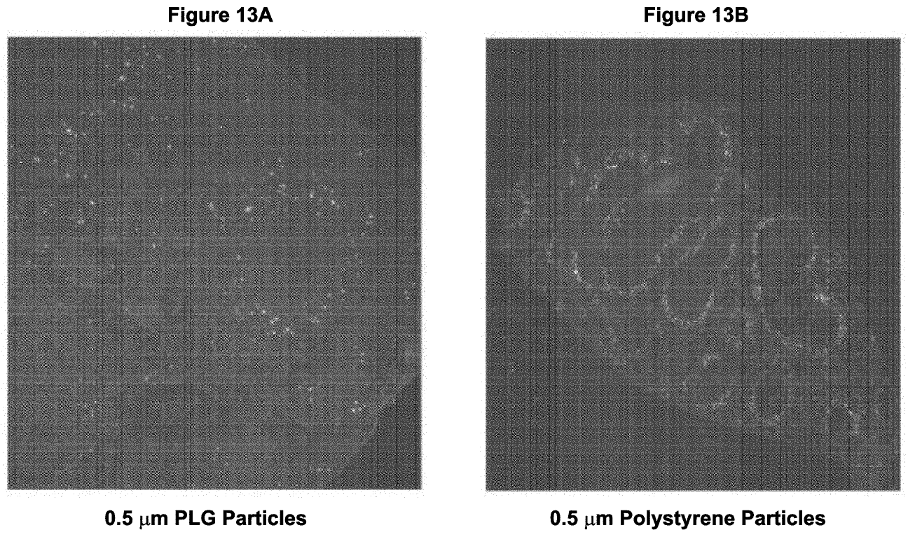

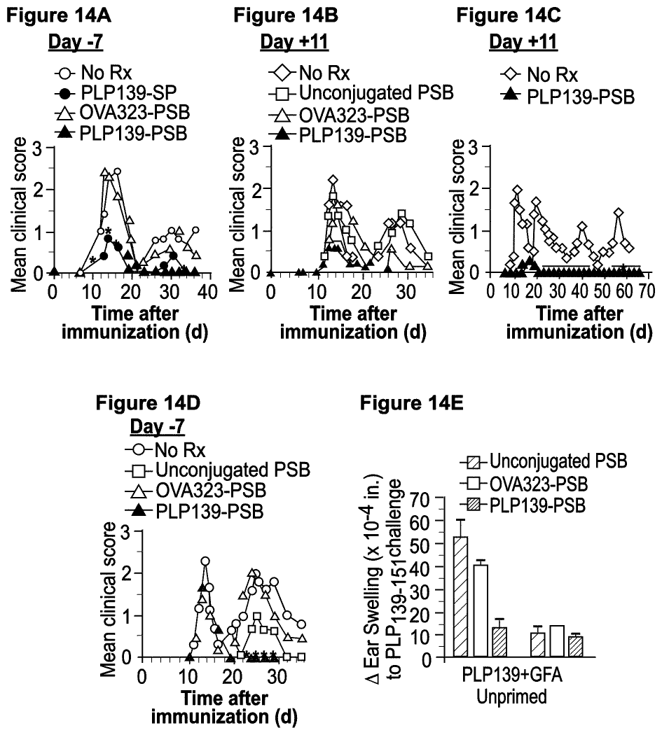

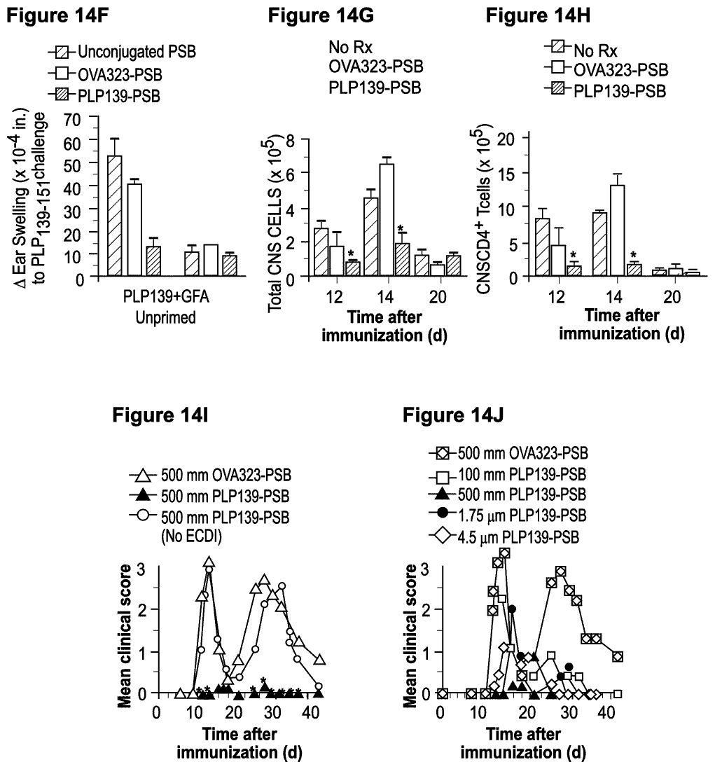

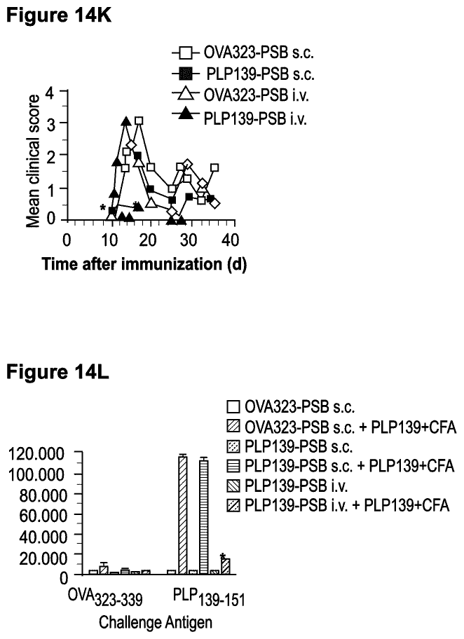

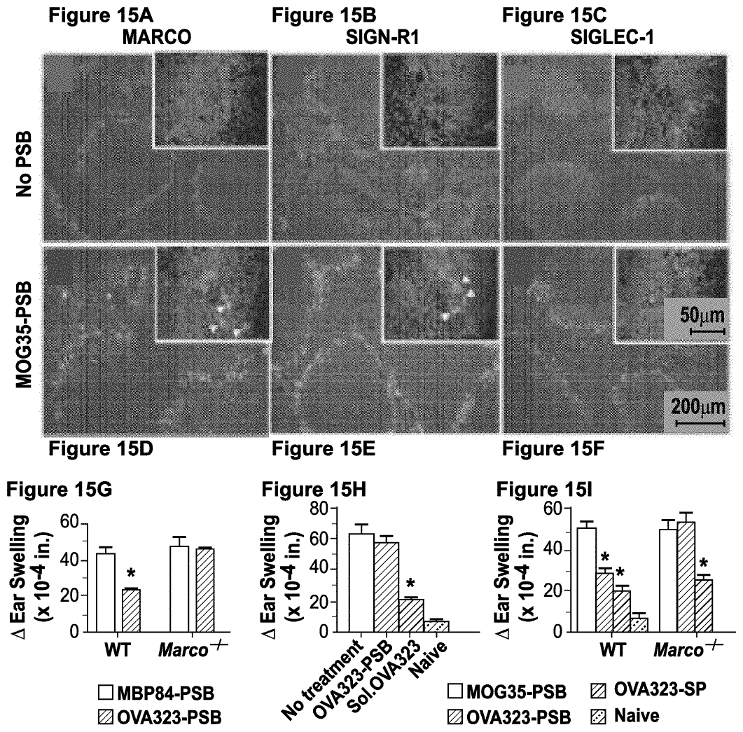

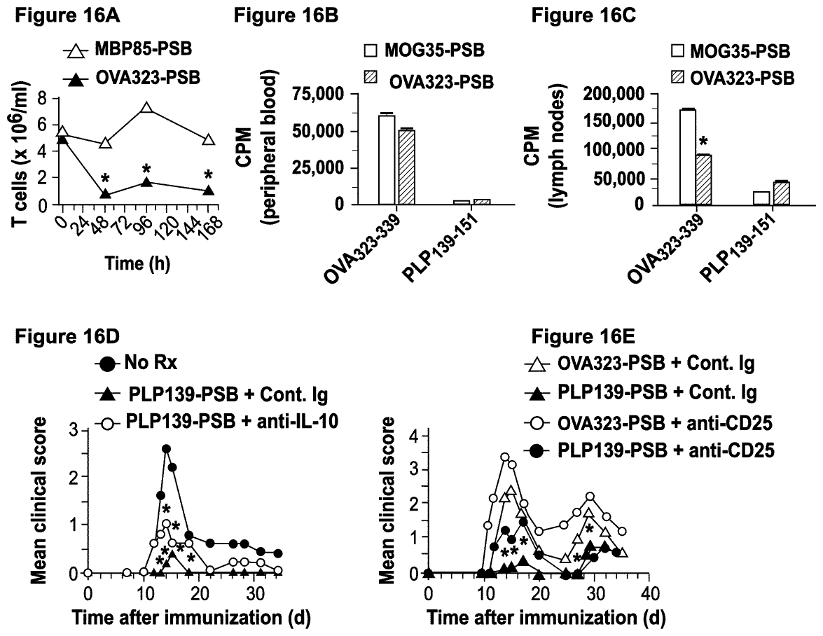

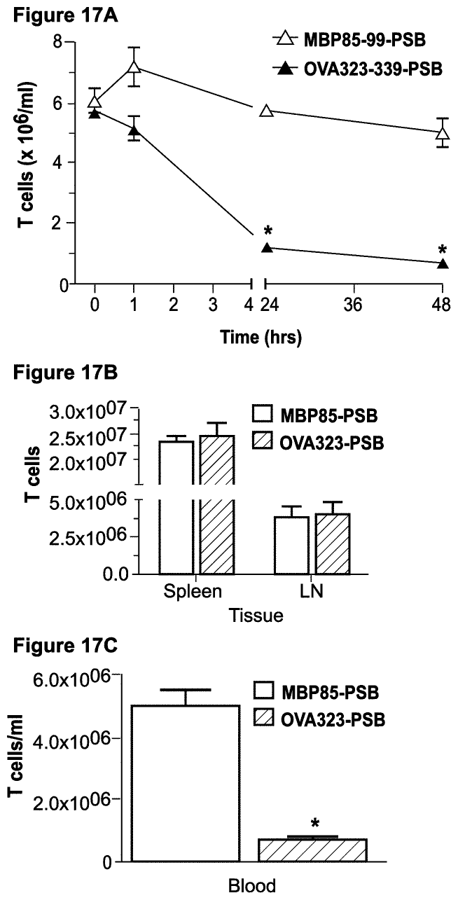

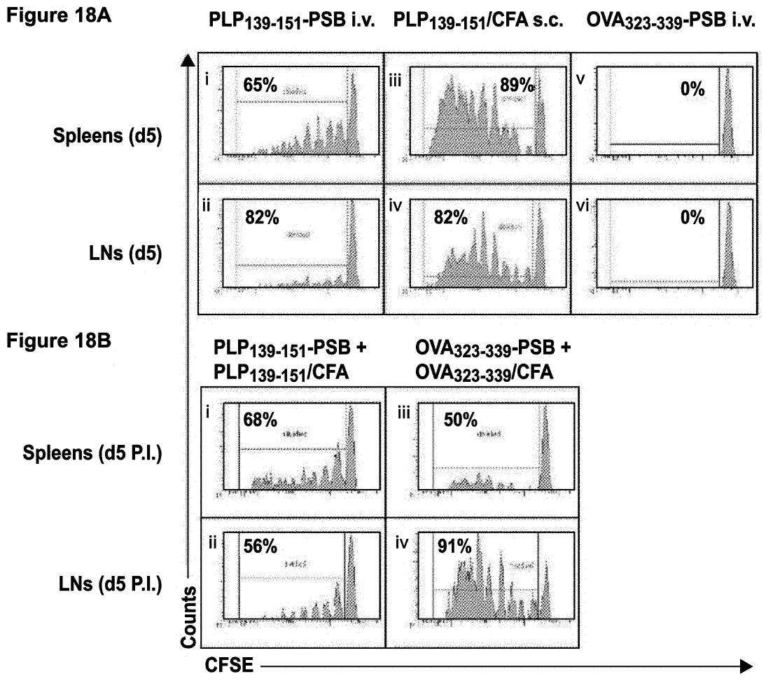

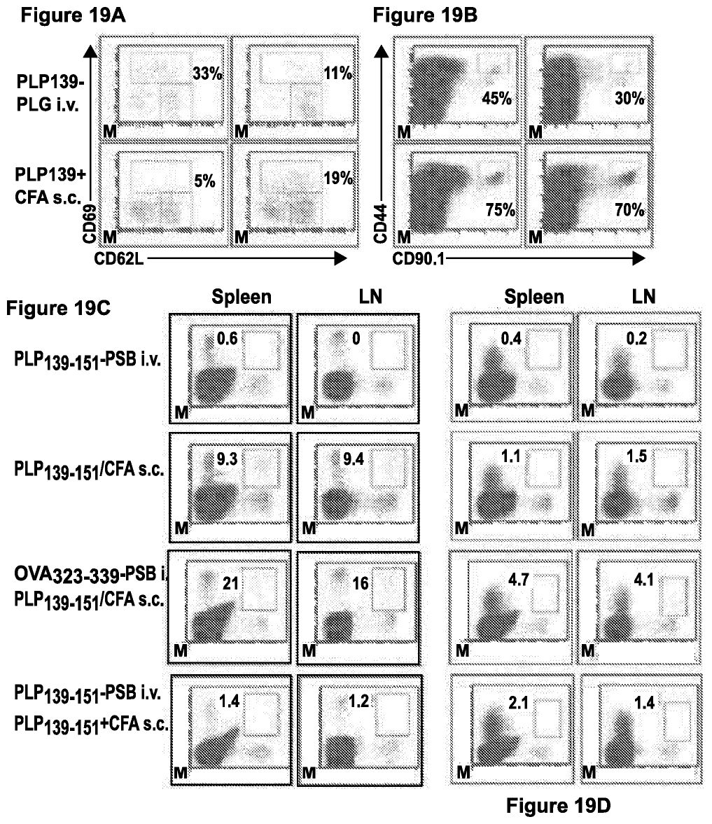

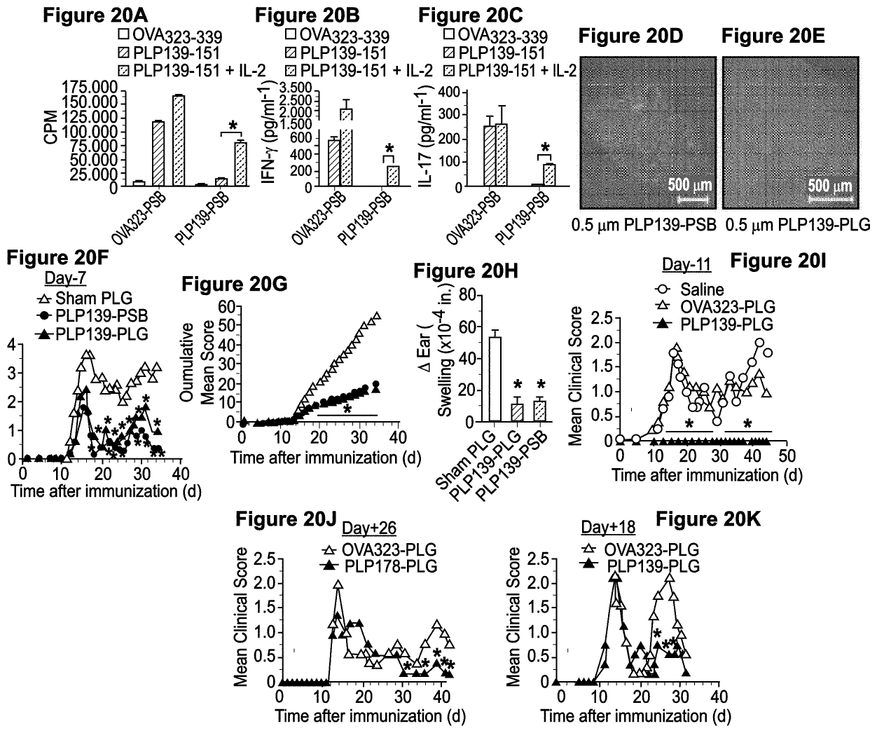

The production of peptide coupled microspheres was performed either as described in Example 16 or Example 20. Either PLP₁₃₉₋₁₅₁ or a control (OVA₃₂₃₋₃₃₉) peptide was coupled to 0.1, 0.5, 0.75 or 4.5 μm polystyrene microspheres. An ECDI-free (NO ECDI) bead mixture was also prepared omitting ECDI coupling. Mice were injected intravenously or subcutaneously with either the PLP₁₃₉₋₁₅₁ or control (OVA₃₂₃₋₃₃₉) peptide bound or ECDI-free microspheres on day 0 relative to priming with PLP₁₃₉₋₁₅₁. Intravenous injection was found to be essential (FIG. 10A). Of the 0.1, 0.5, 0.75 or 4.5 μm PLP₁₃₉₋₁₅₁-coupled microspheres, 0.5 μm microspheres induced the largest amount of tolerance. ECDI-free beads did not induce tolerance at a significant level.

### Example 23. Tolerance Induction Using Peptide-Coupled Polystyrene Microbeads, but not Peptide-Coupled SP Requires the MARCO Scavenger Receptor

Wild type BALB/c and MARCO knockout mice were tolerized with ECDI-coupled polystyrene microspheres with MOG₃₅ (MOG₃₅-MP), with OVA₃₂₃₋₃₃₉ (OVA₃₂₃₋₃₃₉-MP), or ECDI-coupled spelenocytes with OVA₃₂₃₋₃₃₉ (OVA₃₂₃₋₃₃₉-SP). Subsequently, mice were primed with OVA₃₂₃₋₃₃₉ and CFA. ECDI-coupled microspheres were prepared as described in Example 21 and ECDI-coupled splenocytes were prepared as described in Example 1. Control mice were not tolerized or immunized (naïve). The induction of tolerance was tested using the ear swelling test described in Example 3.

The wild type mice demonstrated induced tolerance to the antigen upon being tolerized with either OVA₃₂₃₋₃₃₉-SP or OVA₃₂₃₋₃₃₉-MP (FIG. 11), but the MARCO knockout mice did not exhibit tolerance for the antigen only when tolerized with OVA₃₂₃₋₃₃₉-SP, but not when tolerized with OVA₃₂₃₋₃₃₉-MP (FIG. 11). These results suggest that MARCO is an essential component for microspheres based tolerance induction.

### Example 24. PLP₁₃₉₋₁₅₁-Coupled Polystyrene and PLG Microbeads are Effective in Down-Regulating Induction and Progression of PLP₁₃₉₋₁₅₁ R-EAE

ECDI-coupled polystyrene and PLG microspheres were prepared as described in Examples 16 and 20. Three groups of five R-EAE mice were tolerized with ECDI-coupled polystyrene microspheres with PLP₁₃₉₋₁₅₁, ECDI-coupled PLG microspheres with PLP₁₃₉₋₁₅₁, or with PLG alone on day −7 and immunized on day 0. Mean clinical scores were observed as described in Example 21 up to 40 days post-immunization in each group. The clinical scores are displayed on a daily basis in FIG. 12A and in a cumulative fashion in FIG. 12B. The animals were also tested for ear swelling as described in Example 3.

The results show decreased clinical responses in both the ECDI-coupled polystyrene tolerized mice and in ECDI-coupled PLG tolerized mice. As with the clinical scores, animals tolerized with either of the ECDI-coupled microspheres showed decreased ear swelling compared to the control mice injected with the PLG spheres only. One possible conclusion is that the chemical composition of the microspheres is not a determinant on tolerance induction.

It is understood that the removal of apoptotic debris occurs without immune activation in many examples of normal physiology. Intravenously (i.v.) administered apoptotic cells localize to the splenic marginal zone, where they mediate changes in scavenger receptor expression and upregulate interleukin-10 (IL-10) production. Antigen-coupled apoptotic cell tolerance is associated with the upregulation of IL-10 production by macrophages, induction of T_{reg} cells and co-inhibition of T cells through the CTLA-4 and PD-1 pathways. A particle carrier that localizes to similar areas of the spleen and does not trigger immune activation pathways is therefore useful for various embodiments of the invention. Accordingly, peptide-coupled syngeneic splenic leukocytes (antigen-splenocyte, Ag-SP) can be replaced by inert microparticles covalently linked with antigen.

### Example 25. PLG and Polystyrene Particles Localize to the Splenic Marginal Zone

0.5 μm PLG and polystyrene microspheres were obtained as described in Examples 16 and 20. The particles were fluorescently labeled. Mice were infused with 5×10⁷ polystyrene or PLG microspheres i.v. Spleens were harvested for microsphere localization 3 h later.

Spleens were removed from mice infused with fluorescent-labeled polystyrene or PLG microspheres and fixed in paraformaldehyde for 30 min to 3 h at 4° C. in the dark. Spleens were then frozen in OCT. The blocks were stored at −80° C. in plastic bags to prevent dehydration. Six-micrometer-thick cross-sections were cut on a Reichert-Jung Cryocut CM1850 cryotome (Leica) mounted on Superfrost Plus electrostatically charged slides (Fisher), air dried, and stored at −80° C. Sections were coverslipped with Vectashield mounting medium with DAPI (Vector Laboratories). Slides were examined and images were acquired using a Lica DM5000B fluorescent microscope and Advanced SPOT software. At least eight serial sections from each sample per group were analyzed at original magnification X20, X40, and X100.

Both the PLG and the polystyrene microspheres were found localized to the marginal zone of the spleen (FIGS. 13 A and B), suggesting that the induction of tolerance may be taking place largely in the marginal zone of the spleen.

### Example 26. Ag-PSB Effectively Prevents and Treats EAE

Intravenous administration of soluble peptides cross-linked to syngeneic splenic leukocytes using ECDI (Ag-SP) safely and efficiently induces antigen-specific immune tolerance, is effective in prevention and treatment of TH1/TH17-mediated autoimmune diseases and overcomes many of the draw-backs of failed monoclonal antibody and soluble peptide clinical trials. Overcoming the expense and complexity of GMP isologous leukocyte isolation and peptide coupling would be beneficial for broad clinical application of this therapy. The mechanism of Ag-SP involves the delivery of antigen in the context of apoptotic carrier cells. Inert microparticles covalently linked with antigen were tested as an alternative system to Ag-SP to induce tolerance. We determined the ability of carboxylated 500 nm polystyrene beads (PSB) coupled with the immunodominant proteolipid protein PLP₁₃₉₋₁₅₁ epitope (PLP₁₃₉₋₁₅₁-PSB) to induce tolerance for prevention and treatment of relapsing-remitting EAE (R-EAE). Female SJL/J mice were purchased from Harlan Laboratories (Indianapolis, Ind.). PLP₁₃₉₋₁₅₁ (HSLGKWLGHPDKF), MOG₃₅₋₅₅ (MEVGWYRSPFSRVVHLYRNGK), and OVA₃₂₃₋₃₃₉ (ISQAVHAAHAEINEAGR) were purchased from Genemed Synthesis. PLP₁₇₈₋₁₉₁ (NTWTTCQSIAFPSK) was purchased from Peptides International. SJL/J mice were injected i.v. with 0.5 μm carboxylated polystyrene beads (PSB) coupled to PLP₁₃₉₋₁₅₁ or OVA₃₂₃₋₃₃₉ 7 days prior to EAE initiation by s.c. immunization with PLP₁₃₉₋₁₅₁/Complete Freund's Adjuvant (CFA). A separate group was tolerized with PLP₁₃₉₋₁₅₁-SP. Tolerance was induced by i.v. injection of chemically treated Ag-coupled splenocytes (Ag-SP), as described above. Briefly, spleens were removed from naïve female mice, and the RBCs were lysed. The splenocytes were incubated with ECDI (150 mg/3.2×10⁸ cells; Calbiochem) and peptide (1 mg/ml) in ice, shaking for 1 h. The coupled cells were washed 3× and filtered through a 70 μM cell strainer to remove cell clumps. The Ag-SP were resuspended at 250×10⁶ cells/ml in PBS. Each mouse received 50×10⁶ Ag-SP in 200 μl of PBS given by i.v. injection at the indicated times before disease induction, representing a delivery dosage of a total of 15-20 μg of cell-bound peptide per mouse. Carboxylated polystyrene beads (PBSs) of various diameters were purchased from Polysciences (Warrington, Pa.). Peptide antigens were attached to particles using ethylene-carbodiimide (ECDI) and according to manufacture's instructions (12.5 mg of polystyrene microparticles and 500 μg of peptide in the presence of 10 mg/ml ECDI).

In the R-EAE mouse model of relapsing-remitting multiple sclerosis, SJL/J mice are injected with PLP₁₃₉₋₁₅₁ in adjuvant initiating an autoreactive PLP₁₃₉₋₁₅₁-specifc CD4⁺ T-cell response leading to the primary disease phase characterized by hindlimb paralysis. Mice then spontaneously remit from acute disease, although the tissue damage resulting from the primary response promotes the activation of T cells targeting a second PLP epitope, PLP₁₇₈₋₁₉₁. This phenomenon is termed ‘epitope spreading’ and subsequently causes a second round (relapse) of hind-limb paralysis. In the same way, if mice are initially injected with PLP₁₇₈₋₁₉₁ (the subdominant epitope), PLP₁₃₉₋₁₅₁ will function as the spread epitope. Injection of PLP₁₃₉₋₁₅₁-SP or PLP₁₃₉₋₁₅₁-PSB, but not PSB coupled with an irrelevant ovalbumin peptide (OVA₃₂₃₋₃₃₉), 7 d before the initiation of disease protected the mice from disease (FIG. 14A). Treatment with PLP₁₃₉₋₁₅₁-PSB at the first sign of symptoms (11 d after induction of disease) also prevented disease initiation in the vast majority of mice (FIG. 14B), and this effect lasted for at least 66 d (FIG. 14C). In addition, mice were injected i.v. with 0.5 μm carboxylated polystyrene beads (PSB) coupled to PLP₁₃₉₋₁₅₁, OVA₃₂₃₋₃₃₉ or nothing 7 days prior to PLP₁₇₈₋₁₉₁ EAE induction. Epitope spreading was inhibited as PLP₁₃₉₋₁₅₁-PSB given at day-7 prevented relapse in PLP₁₇₈₋₁₉₁/CFA immunized animals (FIG. 14D). Specific inactivation of myelin-specific CD4⁺ T cells was demonstrated by lack of delayed-type hypersensitivity (DTH) responses to both the immunizing (PLP₁₃₉₋₁₅₁) and spread epitope (PLP₁₇₈₋₁₉₁). Although injection of PLP₁₃₉₋₁₅₁-PSB 7 d before injection of PLP₁₇₈₋₁₉₁ mixed with complete Freund's adjuvant (CFA) did not prevent acute disease, indicating the antigen specificity of the tolerance, it did prevent relapse (FIG. 14D), indicating that this treatment could prevent epitope spreading. Treatment with PLP peptide-coupled PSB affected T-cell function. For example, inactivation of myelin-specific CD4⁺ T cells was shown by a lack of delayed-type hypersensitivity (DTH) responses, an in vivo measure of CD4⁺ T-cell function, to both the immunizing (PLP₁₃₉₋₁₅₁) and spread epitope (PLP₁₇₈₋₁₉₁) (FIGS. 14E & 14F). Selected representative animals from the PLP₁₃₉₋₁₅₁/CFA primed groups in panel 14A (OVA₃₂₃₋₃₃₉-PSB, PLP₁₃₉₋₁₅₁-PSB, and no particles) were ear-challenged with the priming PLP₁₃₉₋₁₅₁ epitope (FIG. 14E) and the PLP₁₇₈₋₁₉₁ spread epitope (FIG. 14F) at 36d post-priming. Additional mice included in this analysis received doses of PSBs i.v., but were not primed for EAE. Ear swelling as a measure of DTH was determined 24 h later and responses to a control OVA₃₂₃₋₃₃₉ peptide were subtracted. DTH was performed via a 24 h ear swelling assay. Pre-challenge ear thickness was determined using Mitutoyo model 7326 engineer's micrometer (Schlesinger's Tools, Brooklyn, N.Y.). Immediately thereafter, DTH responses were elicited by injecting 10 μg of peptide in 10 μl of PBS into the dorsal surface of the ear using 100 μl Hamilton syringe fitted with a 30 gauge needle. The increase in ear thickness over pre-challenge measurements was determined 24 h after ear challenge. Results are expressed in units of 10⁻⁴ inches±SEM.

Treatment with PLP₁₃₉₋₁₅₁-PSB also reduced leukocyte (CD45⁺) and CD4⁺ T-cell infiltration of the CNS (FIGS. 14G & H). SJL/J mice were injected i.v. with 0.5 μm carboxylated PSBs coupled with PLP₁₃₉₋₁₅₁, OVA₃₂₃₋₃₃₉, or nothing 7 days prior to EAE priming with PLP₁₃₉₋₁₅₁/CFA. At the onset of disease (d12), peak of disease (d14), and remission (d20), brains and spinal cords were removed and the number of CD45^{hi} cells (FIG. 14G) and CD3⁺CD4⁺ T cells (FIG. 14H), were enumerated by flow cytometry. Cytometric data were collected on a FACS Canto flow cytometer (Becton-Dickinson). DiVa software was used for date acquisition and analysis (Becton-Dickinson).

The data indicate the ability of Ag-PSB to both prevent and treat EAE in an antigen-specific manner and to avoid anaphylactic responses associated with treatment of mice with established disease tolerized with soluble peptide.

### Example 27. Ag-PSB Tolerance is Dependent on Covalent Peptide Linkage, Particle Size and Route of Administration

The importance of covalent linkage, particle size and route of administration in Ag-PSB tolerance were addressed. SJL/J mice were treated by i.v. injection of 0.5 μM carboxylated PSBs incubated with PLP₁₃₉₋₁₅₁ in the presence of absence of ECDI 7d prior to priming with PLP₁₃₉₋₁₅₁/CFA and monitored for development of clinical disease in comparison to SJL/J mice treated with 0.5 μM OVA₃₂₃₋₃₃₉-PSBs. Covalent linkage with ECDI was found to be essential as PSB incubated with PLP₁₃₉₋₁₅₁ in the absence of ECDI had no impact on disease, which was quantified as described in Example 1 (FIG. 14I). Normalizing for peptide mass (each dose containing 20 μg of peptide), PLP₁₃₉₋₁₅₁ was coupled to particles with varying diameters (FIG. 14J). Accordingly, PSBs of varying diameters (0.1, 0.5, 1.75, and 4.5 μm) were coupled with PLP₁₃₉₋₁₅₁ and i.v. injected into SJL recipients 7d prior to priming with PLP₁₃₉₋₁₅₁/CFA and monitored for development of clinical disease in comparison to SJL/J mice treated i.v. with 0.5 μm OVA₃₂₃₋₃₃₉ PSBs. While 4.5 μm and 1.75 μm diameter particles provided some disease modification, optimal disease protection was conferred by treatment with the standard 0.5 μm PLP₁₃₉₋₁₅₁-PSB, while 0.1 μm PLP₁₃₉₋₁₅₁-PSB prevented relapse but did not confer any protection.

Finally, i.v. administration of Ag-SP has been shown to be critical for efficient tolerance. 0.5 μM PLP₁₃₉₋₁₅₁-PSB or OVA₃₂₃₋₃₃₉-PSB were administered to SJL/J mice via the lateral tail vein (i.v.) or subcutaneously (s.c.) on the flank 7d prior to priming with PLP₁₃₉₋₁₅₁/CFA and monitored for development of clinical disease (FIG. 14K). At d10 post-immunization, spleens and lymph nodes were collected from a subset of the mice shown in Panel 14K and in vitro proliferative responses to stimulation with the PLP₁₃₉₋₁₅₁ priming epitope or a control peptide (OVA₃₂₃₋₃₃₉) determined by [³H]-thymidine uptake. All experiments consisted of 5-10 mice per group and are representative of 2-4 repeats (FIG. 14L). Similar to Ag-SP tolerance, i.v. but not s.c. administration of PLP₁₃₉₋₁₅₁-PSB protects against PLP₁₃₉₋₁₅₁ disease (FIG. 14K) and prevents in vitro recall responses (FIG. 1L).

### Example 28. MARCO Scavenger Receptor Mediates Ag-PSB Uptake and its Critical for Tolerance Induction

The influence of microparticle size and administration route suggests that interactions with phagocytic cells in the splenic marginal zone may be crucial for microparticle-induced tolerance. The 0.5 μm bead diameter and i.v. administration are understood to be physiochemical characteristics that are useful in mediating interactions with phagocytic cells in the splenic marginal zone (MZ). As described above, the infusion of apoptotic debris upregulates the expression of select scavenger receptors, such as MARCO, in the spleen. Scavenger receptors comprise a set of structurally diverse proteins, expressed predominately by phagocytes, that are important in the clearance of modified lipid particles and polyanionic ligands of both host and pathogen origin. The role of the scavenger receptor MARCO in inducing tolerance as a contributor to the uptake and clearance of particulate debris was probed. Mice were infused with PBS (No PSB; FIGS. 15 A-C) or fluorescein isothiocyanate (FITC)-labeled MOG₃₅₋₅₅-PSB (green; FIGS. 15 D-F). Spleens were dissected and snap frozen 18 hours after infusion, and stained for MARCO (FIGS. 15A & D; red), Sign-R1 (FIGS. 15B & E; red), or Siglec-1 (FIGS. 15C & F; red) as well as DAPI (blue). Briefly, 18 hours after infusion, spleens were dissected, fixed in paraformaldehyde for 30 min. to 3 hours at 4° C. in the dart and snap frozen in OCT. The blocks were stored at −80° C. in plastic bags to prevent dehydration. Six-micrometer thick cross-sections were cut on a Reichert-Jung Cryocut CM1850 cryotome (Leica) mounted on Superfrost Plus electrostatically charged slides (Fisher), air-dried, and stored at −80° C. Slides were stained using the Tyramide Signal Amplification Direct kit (NEN) according to the manufacturer's instructions. Nonspecific staining was blocked using either anti-CD16/CD32, (FcIII/IIR, 2.4G2; BD Pharmingen) or 10% horse serum as well as avidin/biotin blocking kit (Vector Laboratories) in addition to the blocking reagent provided by the Tyramide Signal Amplification kit (NEN). Sections were then stained with primary (MARCO, Sign-R1, or Siglec-1) and secondary antibodies as well as DAPI as previously described¹⁵. Sections were coverslipped with Vectashield mounting medium with 4′.6′-diamidone-2-phenylindole (DAPI) (Vector Laboratories) and images were acquired using a Lica DM5000B fluorescent microscope and Advanced SPOT software. At least eight serial sections from each sample per group were analyzed at ×20, ×40 and ×100 magnification.

MOG35-PSB were found to localize in MARCO⁻ (FIG. 15D), Sign-R1⁻ (FIG. 15E) cells, i.e. splenic MZM, but not Siglec-1⁺ marginal zone metallophilic macrophages (MMM) (FIG. 15F).

I.v. administered fluorescein isothiocyanate (FITC)-labeled PLP₁₃₉₋₁₅₁-PSB localized with MARCO⁺ cells in the splenic marginal zone (MZ), presumably the highly phagocytic MZ macrophages (MZM) (FIGS. 15A & D). The cells containing FITC-labeled PLP₁₃₉₋₁₅₁-PSB cells were also shown to express SIGN-R1, the murine homologue of DC-SIGN (FIGS. 15 B & E) that is expressed by MZM professional antigen presenting capabilities (Lyszkiewicz et al., J. Leukoc. Biol. 89, 607-615 (2011); Kang et al., Int. Immunol. 15, 177-186 (2003); Birjandi et al., J. Immunol. 186, 3441-3451 (2011)., but not SIGLEC-1, a marker that defines metallophilic macrophages (MMM) (Backer et al., Proc. Natl. Acad. Sci. USA 107,216-221 (2010); FIGS. 15 C & F). Overall the data indicate that PLP₁₃₉₋₁₅₁-PSB administered i.v. localized to MARCO⁺MZMs. Lyszkiewicz et al., Kang et al., Brijandi et al., and Backer et al., are all herein incorporated by reference in their entirety.

The importance of MARCO in peptide-coupled particle tolerance was confirmed using MARCO-deficient (Marco^{-/-}) BALB/c mice. Female BALB/c mice were purchased from the Jackson Laboratory (Bar Harbor, Me.). Marco^{-/-} mice on the BALB/c background were kindly provided by Dr. Lester Kobzik (Harvard). Wildtype (Wt) or MARCO-deficient BALB/c mice were injected i.v. with OVA₃₂₃₋₃₃₉-PSB or control MBP₈₄₋₁₀₄-PSB 7d prior to immunization with OVA₃₂₃₋₃₃₉/CFA. Eight days post-immunization, animals were ear challenged with OVA₃₂₃₋₃₃₉ or an irrelevant peptide (PLP₁₃₉₋₁₅₁) and ear swelling was measured 24 h later (FIG. 15G). Wt (FIGS. 15G & I) and Marco^{-/-} BALB/c mice (FIGS. 15G-I) were treated i.v. with OVA₃₂₃₋₃₃₉-PSB (FIGS. 15G-I), soluble OVA₃₂₃₋₃₃₉ (FIG. 15H), or OVA₃₂₃₋₃₃₉-SP (FIG. 15I) 7-8d prior to immunization with OVA₃₂₃₋₃₃₉/CFA and DTH responses determined as above. All experiments consisted of 5-10 mice per group and are representative of at least 2-4 separate experiments. Specifically, OVA₃₂₃₋₃₃₉-PSB (the tolerizing peptide) induced significant tolerance as measured by DTH to OVA₃₂₃₋₃₃₉/CFA immunization in wildtype, but not Marco^{-/-} BALB/c mice (FIG. 15G). A role for MARCO in T cell tolerance has not previously been defined. Thus, Marco^{-/-} animals were examined for resistance to other forms of tolerance induction. These mice could be tolerized via infusion of soluble OVA₃₂₃₋₃₃₉ (FIG. 15H) or OVA₃₂₃₋₃₃₉-SP (FIG. 15I). Cumulatively, the data show the importance of MARCO in T cell tolerance induced by Ag-PSB. Further, these finding indicate that soluble peptides and apoptotic Ag-SP induce tolerance through different mechanisms that probably involve different antigen uptake receptors.

### Example 29. Ag-PSB Trigger T Cell Extravasation into Secondary Lymphoid Organs

The temporal and kinetic effects of Ag-PSB infusion on circulating T cells in DO11.10 OVA₃₂₃₋₃₃₉-specific T-cell receptor (TCR) transgenic mice were explored using beads coupled to OVA₃₂₃₋₃₃₉. It is understood that the use of DO11.10 mice for this study is especially suitable for eliminating potential variables related to diverse TCR repertoires and/or potential for self reactivity. T cell receptor transgenic mice expressing a TCR specific for PLP₁₃₉₋₁₅₁ (5B6) on the SJL/J background were the kind gift of Dr. Vijay Kuchroo (Harvard) were bred in-house at Northwestern University. Female D011.10 OVA₃₂₃₋₃₃₉-specific TCR transgenic mice were treated i.v. with 0.5 μM carboxylated PSBs coupled to the cognate peptide (OVA₃₂₃₋₃₃₉) or an irrelevant peptide (MBP₈₅₋₉₉). Draining lymph nodes (LNs) (axillary, brachial, and inguinal) and/or spleens were harvested from naïve mice or primed mice at the indicated days following disease induction, counted, and cultured in 96-well microtiter plates at a density of 5×10⁵ cells/well in a total volume of 200 μl of HL-1 medium (BioWhittaker; 1% penicillin/streptavidin and 1% glutamine). Cells were cultured at 37° C. with medium alone or with different concentrations of peptide Ag for 72 h. During the last 24 h, cultures were pulsed with 1 μCi/well [³H]TdR, and uptake was detected using a Topcount microplate scintillation counter and results are expressed as mean of triplicate cultures. IFN-γ and IL-17 levels were determined by Liqui-Chip analysis. Peripheral blood was analyzed for T cell content at 48, 96, and 168 h post-treatment (FIG. 16A) or at 1, 24, and 48 h post-treatment (FIG. 17 A).

OVA₃₂₃₋₃₃₉-PSB, administered i.v., triggered a rapid and sustained decrease in the number of OVA₃₂₃₋₃₃₉-specific CD4⁺ T cells in the peripheral blood commensurate with a slight increase in the number of T cells in the spleen and lymph nodes (LNs) (FIGS. 16A & 17). This reduction was antigen-specific, as it was not induced by i.v. administration of myelin basic protein (MBP)₈₄₋₉₉-PSB (FIG. 16A).

At 48 h, spleens, lymph nodes and peripheral blood, from select mice were collected and analyzed for OVA₃₂₃₋₃₃₉-specific T cell content by cytometry (FIGS. 17 B & C). The total numbers of CD4⁺KJ-126⁺ cells in the respective tissues is shown (Panel 17B) as well as the numbers of CD4-KJ-126⁺ cells/ml of peripheral blood (Panel 17C) is shown. In addition, 3×10⁵ T cells magnetically purified from peripheral blood and lymph nodes of DO11.10 mice treated i.v. with 0.5 μM carboxylated PSBs coupled to the cognate peptide (OVA₃₂₃₋₃₃₉) or an irrelevant peptide (MOG₃₅₋₅₅) 48 h previously were re-stimulated with 1 μg/ml cognate OVA₃₂₃₋₃₃₉ peptide or PLP₁₃₉₋₁₅₁ in vitro. Proliferation of peripheral blood T cells (FIG. 16B) and peripheral LN T cells (FIG. 16C) measured by [³H]-thymidine uptake is displayed. In addition, T cells in the blood and secondary lymphoid organs of these mice showed functional differences. T cells isolated from the lymph nodes, but not from the blood, of DO11.10 mice that had been i.v. injected with OVA₃₂₃₋₃₃₉-PSB were hypoproliferative in response to OVA₃₂₃₋₃₃₉ (FIGS. 16 B & C).

The data imply that T cell tolerance may require interaction with tolerogenic APC or Treg populations located in the secondary lymphoid organs.

### Example 30. Role of Regulatory T Cells and IL-10 in Ag-PSB Tolerance Induction

Naïve SJL/J mice were treated with control Ig or anti-IL-10 (clone JESS-16E3-200 μg i.p.) (FIG. 16D) or with control Ig or anti-CD25 (clone PC61-500 μg i.p.) (FIG. 16E) one day prior to and one day following treatment with either OVA₃₂₃₋₃₃₉-PSB or PLP₁₃₉₋₁₅₁-PSB. Seven days following tolerization, animals were primed for EAE with PLP₁₃₉₋₁₅₁/CFA and monitored for clinical disease. Induction of tolerance by the i.v. administration of peptides crosslinked to splenic leukocytes is in part dependent on IL-10 (Example 5), and the activity of antigen-specific CD4-CD25⁺Foxp3⁺ Treg cells (Example 7). Neutralization of IL-10 at the time of PLP₁₃₉₋₁₅₁-PSB infusion had only a marginal effect on tolerance induction (FIG. 16D). However, functional inactivation of Tregs by treatment with anti-CD25 prior to PLP₁₃₉₋₁₅₁-PSB infusion resulted in a modest, but significant (P<0.05, analysis of variance (ANOVA)) and reproducible, reversal of the amount of protection during acute R-EAE disease (FIG. 16E). These results suggest that regulatory T cells have an important, but redundant, role in PSB-induced tolerance, as treatment with CD25-specific antibodies only partially blocked tolerance induction. IL-10 seems to make only modest contributions to PSB-induced tolerance.

### Example 31. Ag-PSB Induce Abortive T Cell Activation and Anerqy

The effect of microparticles on T cell proliferation and differentiation was further explored. Carboxyfluorescein diacetate succinimidyl diester (CFSE)-labeled PLP₁₃₉₋₁₅₁-specific transgenic (5B6) T cells were adoptively transferred into naïve SJL/J mice Forty-eight hours later, we i.v. injected 9×10⁹ PLP₁₃₉₋₁₅₁-PSB or OVA₃₂₃₋₃₃₉-PSB or s.c. injected PLP₁₃₉₋₁₅₁ along with CFA. Naïve T cells were isolated from the lymph nodes of healthy 5B6 animals by magnetic separation. Single-cell preparations of nodes were prepared, FcR blocked with 2.4G2, and labeled with CD4⁻ T cell isolation reagents (Miltenyi Biotec). T cells were isolated using an AutoMACS magnetic separator (Miltenyi Biotec). 94-98% purity was routinely achieved. Following isolation, 20×10⁶ T cells/ml were fluorescently labeled in a 4 μM solution of carboxyfluorescein diacetate (CFDA) in PBS for 8 minutes at room temperature. The reaction was quenched by addition of a half volume of heat-inactivated PBS and an additional 5 minute incubation at room temperature. Cells were washed twice in PBS prior to injection into the lateral tail vein of recipient animals (5×10⁶ T cells/recipient). 48 hours following transfer, recipient animals were treated with a variety of antigen-coupled microparticles, or with antigen in CFA. At various times post-treatment, spleens and lymph nodes were isolated and Tg T cells (identifiable by CD90.1 and transgene expression) in these organs were analyzed for cell division and a variety of surface and intracellular markers as described above.

FIG. 18 depicts the findings after naïve CD90.1⁺ PLP₁₃₉₋₁₅₁-specific 5B6 TCR transgenic T cells were sorted from donor lymph nodes, CFSE labeled, and transferred to naïve SJL/J (CD90.2⁺) recipients. 48 h following transfer, recipient animals were treated i.v. with PLP₁₃₉₋₁₅₁-PSB (FIG. 18Ai-ii), primed s.c. with PLP₁₃₉₋₁₅₁/CFA (Panels 18Aiii-iv), or treated i.v. with OVA₃₂₃₋₃₃₉-PSB (FIG. 18Av-vi). Five days following these treatments, spleens and lymph nodes (LNs) were collected and prepared for cytometric analyses of cell division (CFSE dilution). On d5 post-treatment, additional PLP₁₃₉₋₁₅₁-PSB (FIG. 18Bi-ii), and OVA₃₂₃₋₃₃₉-PSB-treated control groups (FIG. 18Biii-iv) were primed with PLP₁₃₉₋₁₅₁/CFA and cytometric analyses of CFSE dilution carried out at d5 post-priming.

PLP₁₃₉₋₁₅₁-specific T cells isolated from the spleen and lymph nodes of PLP₁₃₉₋₁₅₁-PSB-treated mice showed markedly reduced proliferation (CFSE dilution) in terms of both the percentage of total cells divided and the number of divisions per cell compared to cells from mice injected with PLP₁₃₉₋₁₅₁ plus CFA (FIG. 18Ai-Aiv). Notably, this effect was antigen specific, as T cells from mice injected with OVA₃₂₃₋₃₃₉-PSB did not show any CFSE dilution (FIG. 18Av-vi). In addition, a cohort of PLP₁₃₉₋₁₅₁-PSB and OVA₃₂₃₋₃₃₉-PSB treated animals were immunized 5 days after i.v. infusion. We injected a subset of mice first i.v. with PLP₁₃₉₋₁₅₁-PSB or OVA₃₂₃₋₃₃₉-PSB and then s.c. with PLP₁₃₉₋₁₅₁ plus CFA. T cells from mice injected with PLP₁₃₉₋₁₅₁-PSB before PLP₁₃₉₋₁₅₁ plus CFA proliferated less than those from mice injected with PLP₁₃₉₋₁₅₁ plus CFA alone (FIG. 18A, B).

The muted proliferation induced by peptide-coupled particle infusion suggests that tolerance may be induced by abortive T-cell activation.

FIG. 19 depicts the role of naïve T cell activation to direct Ag-PSB engagement and cytokine responses of Ag-PSB tolerized T cells to peptide immunization. Naïve CD90.1⁺ 5B6 TCR transgenic T cells were CFSE labeled and transferred to naïve CD90.2⁺ SJL/J recipients that were then treated i.v. with PLP₁₃₉₋₁₅₁-PSB (FIG. 19A-D) or primed s.c. with PLP₁₃₉₋₁₅₁/CFA (FIG. 19E-H). 5 days following these treatments, spleens and lymph nodes were collected and prepared for cytometric analysis of T cell activation markers CD62L, CD69, and CD44.

FcR blocking with CD16/32 was performed followed by staining with various combinations of the following antibodies: αCD69-APC, αCD69-FITC, αCD62L-APC/AlexaFluor750, αCD44-PE/Cy7, αFoxp3-APC, αFoxp3-PE/Cy7, αCD152-PE, αPD-L1-PE, α1FNγ-PE/Cy7, αIL-17-APC, αCD90.1-Pacific Blue, and αCD45-PE were purchased from eBioscience. αCD25-FITC and αCD25-APC, αCD3-APC-Alexa750, and αCD4-PerCP were purchased from Becton-Dickinson. Cytometric data were collected on a FACS Canto flow cytometer (Becton-Dickinson). DiVa software was used for data acquisition and analysis (Becton-Dickinson). Transgenic T cells were identified by CD90.1 and CFSE signals.

Cytokine responses of Ag-PSB tolerized T cells to peptide immunization were also monitored. Naïve CD90.1⁺ 5B6 TCR Tg T cells were CFSE labeled and transferred to naïve CD90.2⁺ SJL/J recipients that were then treated i.v. with PLP₁₃₉₋₁₅₁-PSB (FIGS. 19I-L) or primed s.c. with PLP₁₃₉₋₁₅₁/CFA (FIGS. 19M-P). 5 days following treatment, spleen and lymph node cell preparations were stimulated with phorbol 12-myristate 13-acetate (PMA) and ionomycin for 5 hours prior to intracellular staining for IL-17A (Left Panels) and IFN-γ (Right Panels).

SJL/J recipients of naïve CFSE-labeled 5B6 TCR Tg T cells were treated i.v.OVA₃₂₃₋₃₃₉-PSB or PLP₁₃₉₋₁₅₁-PSB and subsequently primed with PLP₁₃₉₋₁₅₁/CFA 5 days later (FIGS. 19Q-X). 5 days following priming, spleen and lymph node preparations were stimulated with PMA and ionomycin for 5 hours prior to intracellular staining for IL-17A (Left Panels) and IFN-γ (Right Panels).

After adoptive transfer into naive SJL/J mice, PLP₁₃₉₋₁₅₁-specific T cells isolated from mice injected i.v. with PLP₁₃₉₋₁₅₁-PSB showed classical T-cell activation phenotypes: upregulated expression of CD69 and CD44 and downregulated expression of CD62L (FIG. 19A, B). Notably, a larger fraction of splenic PLP₁₃₉₋₁₅₁-specific T cells expressed CD69 in mice injected i.v. with PLP₁₃₉₋₁₅₁-PSB than in mice injected s.c. with PLP plus CFA (FIG. 19A). However, the opposite was true with regard to CD44 expression (FIG. 19B). In addition, T cells in mice i.v. administered PLP₁₃₉₋₁₅₁-PSB produced neither interferon-γ (IFN-γ) nor IL-17, whereas cells in mice injected s.c. with PLP₁₃₉₋₁₅₁ and CFA produced both of these cytokines (FIG. 19C, D). Furthermore, T cells from mice injected i.v. with PLP₁₃₉₋₁₅₁-PSB produced minimal IL-17 and IFN-γ, even when the recipient mice were challenged s.c. with PLP₁₃₉₋₁₅₁ plus CFA 5 d later (FIG. 19C, D); as shown by OVA₃₂₃₋₃₃₉ injection, this effect was antigen specific. Together these findings suggest that PLP₁₃₉₋₁₅₁-PSB induce abortive T-cell activation.

### Example 32. Peptide-Coupled Particles Induce T Cell Anergy

A potential role for clonal anergy in PLP₁₃₉₋₁₅₁-PSB tolerance was further examined. SJL/J mice were treated i.v. with OVA₃₂₃₋₃₃₉-PSB or PLP₁₃₉₋₁₅₁-PSB 7 days prior to priming with PLP₁₃₉₋₁₅₁/CFA s.c. 8 days following priming, proliferative in vitro responses of spleen and lymph nodes were measured in response to stimulation with the priming antigen (PLP₁₃₉₋₁₅₁) or a control antigen (OVA₃₂₃₋₃₃₉) in the presence of absence of 200 U/ml of exogenous IL-2 (FIG. 20A). Supernatants were collected for measurements of secreted IFN-γ (FIG. 20B) and IL-17A (FIG. 20C).

Compared to T cells from OVA₃₂₃₋₃₃₉-PSB-injected mice, those from PLP₁₃₉₋₁₅₁-PSB-injected mice showed less proliferation and IFN-γ and IL-17 production after in vitro re-stimulation with PLP₁₃₉₋₁₅₁. However, these responses were significantly (P<0.01, ANOVA₃₂₃₋₃₃₉), but not completely, restored by the addition of exogenous IL-2 to the cultures. (FIGS. 20A-C). Thus, antigenic peptides coupled to microparticles are capable of inducing T cell anergy, which can be overcome in vitro through the addition of exogenous IL-2.

### Example 33. Peptide De-Coupled Biodegradable Poly(Lactide-Co-Glycolide (PLG) Microparticles Induce T Cell Anergy

Polystyrene microparticles are able to serve as surrogates of apoptotic debris and to serve as antigen carriers for efficient tolerance induction. Tolerance induction was further determined with biocompatible, biodegradable microparticles. Particles made of the US Food and Drug Administration approved material poly(lactide-co-glycolide) (PLG) were tested. PLG is stable and is considered to be immunologically inert. 0.5 μM carboxylated PLG microparticles were purchased from Phosphorex, Inc. (Hopkinton, Mass.) and peptide antigens attached using ECDI exactly as for the PSBs in Example 26. Animals received intravenous injections of approximately 9×10⁹ microparticles comprising 15-20 μg of peptide, depending on the sequence used in the coupling reaction.

SJL/J mice were treated with 0.5 μm FITC-PSBs (FIG. 20D) or 0.5 μm biodegradable FITC-PLG microparticles coupled with PLP₁₃₉₋₁₅₁ (FIG. 20E). Frozen spleen sections were prepared, from a subset of animals, 12 h later and counterstained with DAPI (blue). 7 days after infusion the tolerized and control groups were primed with PLP₁₃₉₋₁₅₁/CFA and monitored for development of clinical disease by assessing mean clinical score (FIG. 20F) and cumulative mean clinical score over time (FIG. 20G).

Similarly to PLP₁₃₉₋₁₅₁-PSB, PLP₁₃₉₋₁₅₁-PLG administered i.v. localized to the splenic marginal zone (FIGS. 20D & E). When administered prophylactically, PLP₁₃₉₋₁₅₁-PLG administered i.v. reduced EAE clinical scores and PLP₁₃₉₋₁₅₁-specific DTH responses (FIG. 20F-H). Ongoing EAE disease was also treated with i.v. administration of PLP₁₃₉₋₁₅₁-PLG. (FIG. 20I).

Microparticles coupled with the appropriate relapse-associated myelin epitopes were tested for their ability to inhibit disease relapse when administered during disease remission. Intravenous administration of PLP₁₃₉₋₁₅₁-PLG 25 d after s.c. injection of PLP₁₃₉₋₁₅₁ and CFA reduced the severity of relapse symptoms (FIG. 20J), as did infusion of PLP₁₃₉₋₁₅₁-PLG 18 d after s.c. injection of PLP₁₃₉₋₁₅₁ and CFA (FIG. 20K).

### Example 34. Administration of Peptide Coupled PLG Microspheres for Tolerance Induction to a Human Subject with a Gluten Allergy or Celiac Disease

Celiac disease and gluten allergy are generally characterized by an immune response to wheat gluten proteins such as gliadin proteins and glutenin. Symptoms can include pain and/or discomfort of the digestive track, failure to thrive, fatigue, and has been linked to cancer risk.

Fluoresbrite® YG Carboxylate microspheres are purchased from Polysciences, Inc. (Warrington, Pa.). The microspheres are coupled with at least two antigenic peptides corresponding to the α-, γ-, ω-gliadins and/or to glutenin according to the following protocol. The sequences of the antigenic peptides can comprise the sequences listed in Table 1.

TABLE 1
List of celiac disease relevant T-cell epitopes recognized by CD4⁺ T cells

| | | Peptide-binding register | | | | | | | | | |
|---|---|---|---|---|---|---|---|---|---|---|---|
| Epitope^{a} | Previous names | 1 | 2 | 3 | 4 | 5 | 6 | 7 | 8 | 9 | Reference |

DQ2.5 restricted epitopes

| | | | | | | | | | | | |
|---|---|---|---|---|---|---|---|---|---|---|---|
| DQ2.5-glia-α1a | DQ2-α-I, α9 | P | F | P | Q | P | E | L | P | Y | (Arentz-Hansen et al., 2000) |
| DQ2.5-glia-α1b | DQ2-α-III | P | Y | P | Q | P | E | L | P | Y | (Arentz-Hansen et al., 2002) |
| DQ2.5-glia-α2 | DQ2-α-II, α2 | P | Q | P | E | L | P | Y | P | Q | (Arentz-Hansen et al., 2000) |
| DQ2.5-glia-α3 | glia-α20 | F | R | P | E | Q | P | Y | P | Q | (Vader et al., 2002b) |
| DQ2.5-glia-γ1 | DQ2-γ-I | P | Q | Q | S | F | P | E | Q | Q | (Sjöström et al., 1998) |
| DQ2.5-glia-γ2 | DQ2-γ-II, γ30 | I | Q | P | E | Q | P | A | Q | L | (Qiao et al., 2005; Vader et al., |
| | | | | | | | | | | | 2002b) |
| DQ2.5-glia-γ3 | DQ2-γ-III | Q | Q | P | E | Q | P | Y | P | Q | (Arentz-Hansen et al., 2002) |
| DQ2.5-glia-γ4a | DQ2-γ-IV | S | Q | P | E | Q | E | F | P | Q | (Arentz-Hansen et al., 2002) |
| DQ2.5-glia-γ4b | DQ2-γ-VIIc | P | Q | P | E | Q | E | F | P | Q | (Qiao et al., 2005) |
| DQ2.5-glia-γ4c | DQ2-γ-VIIa | Q | Q | P | E | Q | P | F | P | Q | (Arentz-Hansen et al., 2002) |
| DQ2.5-glia-γ4d | DQ2-γ-VIIb | P | Q | P | E | Q | P | F | C | Q | (Qiao, unpublished) |
| DQ2.5-glia-γ5 | DQ2-γ-VI | Q | Q | P | F | P | E | Q | P | Q | (Arentz-Hansen et al., 2002) |
| DQ2.5-glia-ω1 | DQ2-ω-1 | P | F | P | Q | P | E | Q | P | F | (Tye-Din et al., 2010) |
| DQ2.5-glia-ω2 | DQ2-ω-11 | P | Q | P | E | Q | P | F | P | W | (Tye-Din et al., 2010) |
| DQ2.5-glut-L1 | Glutenin-17 | P | F | S | E | Q | E | Q | P | V | (Vader et al., 2002b) |
| DQ2.5-glut-L2 | Glutenin-156 | F | S | Q | Q | Q | E | S | P | F | (Stepniak et al., 2005; Vader et |
| | | | | | | | | | | | al., 2002b) |
| DQ2.5-hor-1 | Hor-α9, Hα9 | P | F | P | Q | P | E | Q | P | F | (Tye-Din et al., 2010; Vader et |
| | | | | | | | | | | | al., 2003a) |
| DQ2.5-hor-2 | Hor-α2, Hα2 | P | Q | P | E | Q | P | F | P | Q | (Vader et al., 2003a) |
| DQ2.5-hor-3 | hor-I-DQ2 | P | I | P | E | Q | P | Q | P | Y | (Tye-Din et al., 2010) |
| DQ2.5-sec-1 | Sec-α9, Sα9 | P | F | P | Q | P | E | Q | P | F | (Tye-Din et al., 2010; Vader et |
| | | | | | | | | | | | al., 2003a) |
| DQ2.5-sec-2 | Sec-α2, Sα2 | P | Q | P | E | Q | P | F | P | Q | (Vader et al., 2003a) |
| DQ2.5-ave-1a | Av-α9A | P | Y | P | E | Q | E | E | P | F | (Arentz-Hansen et al., 2004; |
| | | | | | | | | | | | Vader et al., 2003a) |
| DQ2.5-ave-1b | Av-α9D, 1490 | P | Y | P | E | Q | E | Q | P | F | (Arentz-Hansen et al., 2004; |
| | | | | | | | | | | | Vader et al., 2003a) |

DQ2.2-restricted epitopes

| | | | | | | | | | | | |
|---|---|---|---|---|---|---|---|---|---|---|---|
| DQ2.2-glut-L1 | glutenin-17 | P | F | S | E | Q | E | Q | P | V | (Bodd et al., 2012) |

DQ8 restricted epitopes

| | | | | | | | | | | | |
|---|---|---|---|---|---|---|---|---|---|---|---|
| DQ8-glia-α1 | DQ8-α-I | E | G | S | F | Q | P | S | Q | E | (van de Wal et al., 1998b) |
| DQ8-glia-γ1a | DQ8-γ-Ia | E | Q | P | Q | Q | P | F | P | Q | (Tollefsen et al., 2006) |
| DQ8-glia-γ1b | DQ8-γ-Ib | E | Q | P | Q | Q | P | Y | P | E | (Tollefsen et al., 2006) |
| DQ8-glut-H1 | HMW-glutenin | Q | G | Y | Y | P | T | S | P | Q | (van de Wal el al., 1999) |

DQ8.5 restricted epitopes

| | | | | | | | | | | | |
|---|---|---|---|---|---|---|---|---|---|---|---|
| DQ8.5-glia-α1 | DQ8-α-I | E | G | S | F | Q | P | S | Q | E | (Kooy-Winkelaar et al., 2011) |
| DQ8.5-glia-γI | | P | Q | Q | S | F | P | E | Q | E | (Kooy-Winkelaar et al., 2011) |
| DQ8.5-glut-HI | HMW-glutenin | Q | G | Y | Y | P | T | S | P | Q | (Kooy-Winkelaar et al., 2011) |

^{a}In the epitope names, these short terms are used to denote the type of proteins that the epitopes derive from: ‘glia-α’ denotes α-gliadin, ‘glia-γ’ denotes γ-gliadin, ‘glia-ω’ denotes ω-gliadin, ‘glut-L’ denotes low molecular weight glutenin, ‘glut-H’ denotes high molecular weight glutenin, ‘hor’ denotes hordein, ‘sec’ denotes secalin and ‘ave’ denotes avenin.

The peptides are coupled to the microspheres using ECDI to a specific number of active amino or carboxyl sites on the particles. Microspheres are washed 2× in PBS, resuspended at 3.2×106/ml in PBS with 1 mg/ml of each peptide and 30.75 mg/ml ECDI (CalBiochem, La Jolla, Calif.), and incubated for 1 h at 4° C. with periodic shaking. Peptide-coupled microspheres are then washed 3× in PBS, filtered through a 70 μm cell strainer, and resuspended at 250×106 microspheres/ml in PBS. Cocktails of microspheres coupled with combinations of two, three, or more peptides are prepared. In separate batches, microspheres are also coupled with a single antigenic peptide each. Cocktails of the singly coupled microspheres are also prepared in different combinations.

The resulting cocktails are administered i.v. to a human subject diagnosed with or suspected of having a gluten allergy. The subject is monitored before and after application (e.g., 1 day, 1 week, 1 month, 6 months, 1 year after application) to demonstrate the efficacy of the procedure in terms of reduction of gluten allergy symptoms. Subjects are observed for symptoms associated with gluten allergy or celiac disease that are known in the art.

### Example 35: Administration of Peptide Coupled PLG Microspheres for Tolerance Induction to a Human Subject Receiving a Tissue Transplant

Fluoresbrite® YG Carboxylate microspheres are purchased from Polysciences, Inc. (Warrington, Pa.). The microspheres are coupled with one or more antigenic peptides isolated from tissue isolated from a donor subject, the tissue to be transplanted into a host subject. The antigenic peptides are isolated using means known to those of skill in the art. The peptides are coupled to the microspheres using ECDI to a specific number of active amino or carboxyl sites on the particles. Microspheres are washed 2× in PBS, resuspended at 3.2×106/ml in PBS with 1 mg/ml of each peptide and 30.75 mg/ml ECDI (CalBiochem, La Jolla, Calif.), and incubated for 1 h at 4° C. with periodic shaking. Peptide-coupled microspheres are then washed 3× in PBS, filtered through a 70 μm cell strainer, and resuspended at 250×106 microspheres/ml in PBS. Cocktails of microspheres coupled with combinations of two or more peptides are prepared. In separate batches, microspheres are also coupled with a single antigenic peptide each. Cocktails of the singly coupled microspheres are also prepared in different combinations.

The resulting cocktails are administered i.v. to the host subject upon receiving the tissue transplant from the donor. The host subject is observed for clinical symptoms associated with transplant rejection using methods that are known in the art. Clinical symptoms and indicators of transplant rejection are reduced after administration of the cocktails.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives in the embodiments of the invention described herein may be employed in practicing the invention. It is intended that the following claims define the scope of the invention and that methods and structures within the scope of these claims and their equivalents be covered thereby.

1. A method of inducing antigen-specific tolerance in a subject suffering from or at risk of an autoimmune disease, transplant rejection, or allergy comprising intravenously administering a composition to said subject, wherein said composition comprises an apoptotic body surrogate and a plurality of immunodominant epitopes associated with one or more antigens suspected to cause said autoimmune disease, transplant rejection, or allergy.

2-8. (canceled)

9. The method of claim 1, wherein said autoimmune disease is multiple sclerosis.

10. The method of claim 1, wherein said plurality of immunodominant epitopes is attached to said apoptotic body surrogate.

11. The method of claim 1, wherein said plurality of immunodominant epitopes is attached to a plurality of apoptotic body surrogates.

12. (canceled)

13. The method of claim 1, wherein said composition is administered subsequent to said subject's exposure to said antigen.

14. The method of claim 1, wherein said administering is concurrent with onset of said autoimmune disease, transplant rejection, or allergy.

15. The method of claim 1, wherein said administering is subsequent to onset of said autoimmune disease, transplant rejection, or allergy.

16. The method of claim 1, wherein said administering prevents relapse of said autoimmune disease, transplant rejection, or allergy.

17-18. (canceled)

19. The method of claim 1, wherein said subject has previously had an adverse reaction to said one or more antigens.

20-124. (canceled)

125. The method of claim 1, wherein the apoptotic body surrogate is a leukocyte.

126. The method of claim 125, wherein the said leukocyte comprises a syngeneic splenic leukocyte.

127. The method of claim 1, wherein the said plurality of immunodominant epitopes are attached to the apoptotic body surrogate by crosslinking.

128. The method of claim 1, wherein the said plurality of immunodominant epitopes comprise a polypeptide, polynucleotide, carbohydrate, glycolipid, or any combinations thereof.
